SEQUENCE LISTING (17 sequences)

SEQ ID NO: 1 - DNA, 1917 bp, Homo sapiens
gagaattgctcctgccctgggaagaggctcagcacagaaagaggaaggacagcacagctgacagccgtgctcagagagtttctggatcctaggcttatctccacagaggagaacacacaagcagcagagaccatgggaaccctctcagcccctccctgcacacagcgcatcaaatggaaggggctcctgctcacagcatcacttttaaacttctggaacctgcccaccactgcccaagtcacgattgaagccgagccaaccaaagtttccgaggggaaggatgttcttctacttgtccacaatttgccccagaatcttaccggctacatctggtacaaagggcaaatgagggacctctaccattacattacatcatatgtagtagacggtgaaataattatatatgggcctgcatatagtggacgagaaacagcatattccaatgcatccctgctgatccagaatgtcacccgggaggacgcaggatcctacaccttacacatcataaagggagatgatgggactagaggagtaactggacgtttcaccttcaccttacacctggagactcctaagccctccatctccagcagcaacttaaatcccagggagaccatggaggctgtgagcttaacctgtgaccctgagactccagacgcaagctacctgtggtggatgaatggtcagagcctccctatgactcacagcttgaagctgtccgaaaccaacaggaccctctttctattgggtgtcacaaagtatactgcaggaccctatgaatgtgaaatacggaacccagtgagtgccagccgcagtgacccagtcaccctgaatctcctcccgaagctgcccaagccctacatcaccatcaacaacttaaaccccagggagaataaggatgtcttaaacttcacctgtgaacctaagagtgagaactacacctacatttggtggctaaatggtcagagcctcccggtcagtcccagggtaaagcgacccattgaaaacaggatcctcattctacccagtgtcacgagaaatgaaacaggaccctatcaatgtgaaatacgggaccgatatggtggcatccgcagtgacccagtcaccctgaatgtcctctatggtccagacctccccagaatttacccttcattcacctattaccgttcaggagaagtcctctacttgtcctgttctgcggactctaacccaccggcacagtattcttggacaattaatgaaaagtttcagctaccaggacaaaagctctttatccgccatattactacaaagcatagcgggctctatgtttgctctgttcgtaactcagccactggcaaggaaagctccaaatccatgacagtcgaagtctctgcttatagcagttcaataaactatacttctgggaaccgtaattgaaacatttacttttgctttctacctgactgccccagaattgggcaactattcatgagaattgatatgtttatggtaatacacatatttgcacaagtacagtaacaatctgctttctttgtaacatgacacatttgaaatcattggttatattaccaatgctttgattcggatgttatattaaaaacatagatagaatgaaccaatatgaactgcaggcaaagtctgaagtcagccttggtttggcttcctattctcaagaggtttgtgaagatttaatctcagattccttataaaaacttagagaaaagaaaattttagaagacagcctacatggtccattgctactcttgctgcacttatgtaaacaatcagaccacatttgaagaaactccacctattttgcaaacaaacttattctactgaaattatcattggtaaaagtagagatgcccatagagggaaaaattatgtggaaaataaaaactgtagtatacctgt

SEQ ID NO: 2 - DNA, 1573 bp, Homo sapiens
gggaattgctgctgccccaggaagaggctcagtgcagaaggaggaaggacagcacagctgacagccgtgctcaggaagtttctggatcctaggctcatctccacagaggagaacacacaggcagcagagaccatggggcccctctcagcccctccctgcacagagcacatcaaatggaaggggctcctggtcacagcatcacttttaaacttctggaacctgcccaccactgcccaagtcacgattgaagcccagccaccaaaagtttccgaggggaaggatgttcttctacttgtccacaatttgccccagaatcttactggctacatctggtacaaagggcaaatcagggacctctaccattacattacatcatatgtagtagacggtcaaataattatatatgggcctgcatatagtggacgagaaacagcatattccaatgcatccctgctgatccagaatgtcacccgggaggacgcaggatcctacaccttacacatcataaagcgaggtgatgggactagaggagtaactggatatttcaccttcaccttatacctggagactcccaagccctccatctccagcagcaacttaaaccccagggaggccatggaaactgtgatcttaacctgtgatcctgagactccggacacaagctaccagtggtggatgaatggtcagagcctccctatgactcataggtttcagctgtccgaaaccaacaggaccctctttctatttggtgtcacaaagtatactgcaggaccctatgaatgtgaaatacggaactcagggagtgccagccgcagtgacccagtcaccctgaatctcctccatggtccagacctccccagaattcacccttcatacaccaattaccgttcaggagataacctctacttgtcttgcttcgcgaactctaacccaccggcacagtattcttggacaattaatgggaagtttcagcaatcaggacaaaatctgtttatcccccaaattactacaaagcatagcgggctctatgtttgctctgttcgtaactcagccactggcgaggaaagctccacatcgttgacagtcaaagtctctgcttctacaagaataggacttcttcctctccttaatccaacatagcagctgtgatgtcatttctgtatttcaggaagactggcaggagatttatggaaaggtctcttacaaggactcttgaatacaagctcctgataacttcaagatcataccactggactaagaactttcaaaattttaatgaacaggctgataccttcatgaaattcaagacaaagaagaaaaatactcaatgttattggactaaataatcaaaaggataatgatttcataattttctatttgaaaatgtgctgattcttggaatgtttcattctccagatttatgaacattttttcttgagcaattggtaaagtatacttttgtaaacaaaaattgaaacatttccttttgctctctatctgagtgccccagaattgggaatctattcatgagtattcatatgtttatggtaataaagctatttgcacaagttca

SEQ ID NO: 3 - DNA, 1873 bp, Homo sapiens
agaaggaggaaggacagcacagctgagagccatgctcaggaagtttctggatcctaggctcagctccacagaggagaacacgcaggcagcagagaccatggggcccctctcagcccctccctgcacacagcgcatcacctggaaggggctcctgctcacagcattacttttaaacttctggaacttgcctaccactgcccaagtcacgattgaagccgagccaaccaaagtttccaaggggaaggacgttcttctacttgtccacaatttgccccagaatcttgctggctacatctggtacaaagggcaaatgaaggacctctaccattacattacatcatacgtagtagatggtcaaataattatatatgggcctgcatacagtggacgagaaacagtatattccaatgcatccctgctgatccagaatgtcacccgggaggacgcaggatcctacaccttacacatcgtaaagcgaggtgatgggactagaggagaaactggacatttcaccttcaccttatacctggagactcccaagccctccatctccagcagcaacttataccccagggaggacatggaggctgtgagcttaacctgtgatcctgagactccggacgcaagctacctgtggtggatgaatggtcagagcctccctatgactcacagcttgcagttgtccaaaaacaaaaggaccctctttctatttggtgtcacaaagtacactgcaggaccctatgaatgtgaaatacggaacccagtgagtgccagccgcagtgacccagtcaccctgaatctcctcccgaagctgcccaagccctacatcaccatcaacaacttaaaccccagggagaataaggatgtcttagccttcacctgtgaacctaagagtgagaactacacctacatttggtggctaaatggtcagagcctcccggtcagtcccagggtaaagcgacccattgaaaacaggatcctcattctacccagtgtcacgagaaatgaaacaggaccctatcaatgtgaaatacaggaccgatatggtggcatccgcagttacccagtcaccctgaatgtcctctatggtccagacctccccagaatttacccttcattcacctattaccattcaggagaaaacctctacttgtcctgcttcgcggactctaacccaccagcagaatattcttggacaattaatgggaagtttcagctatcaggacaaaagctctttatcccccagattactacaaagcatagcgggctctatgcttgctctgttcgtaactcagccactggcatggaaagctccaaatccatgacagtcaaagtctctgctccttcaggaacaggacatcttcctggccttaatccattatagcagccgtgatgtcatttctgtatttcaggaagactggcagacagttgctttcattcttcctcaaagtatttaccatcagctacagtccaaaattgctttttgttcaaggagatttatgaaaagactctgacaaggactcttgaatacaagttcctgataacttcaagatcataccactggactaagaactttcaaaattttaatgaacaggctgatacttcatgaaattcaagacaaagaaaaaaacccaattttattggactaaatagtcaaaacaatgttttcataattttctatttgaaaatgtgctgattctttgaatgttttattctccagatttatgcactttttttcttcagcaattggtaaagtatacttttgtaaacaaaaattgaaacatttgcttttgctccctaagtgccccagaattgggaaactattcaggagtattcatatgtttatggtaataaagttatctgcacaagttca

SEQ ID NO: 4 - DNA, 2032 bp, Homo sapiens
agcacagaaggaggaaggacagcacagctgacagccgtactcaggaagcttctggatcctaggcttatctccacagaggagaacacacaagcagcagagaccatggggcccctctcagcccctccctgcacacagcgcatcacctggaagggggtcctgctcacagcatcacttttaaacttctggaatccgcccacaactgcccaagtcacgattgaagcccagccacccaaagtttctgaggggaaggatgttcttctacttgtccacaatttgccccagaatcttgctggctacatttggtacaaagggcaaatgacatacctctaccattacattacatcatatgtagtagacggtcaaagaattatatatgggcctgcatacagtggaagagaaagagtatattccaatgcatccctgctgatccagaatgtcacgcaggaggatgcaggatcctacaccttacacatcataaagcgacgcgatgggactggaggagtaactggacatttcaccttcaccttacacctggagactcccaagccctccatctccagcagcaacttaaatcccagggaggccatggaggctgtgatcttaacctgtgatcctgcgactccagccgcaagctaccagtggtggatgaatggtcagagcctccctatgactcacaggttgcagctgtccaaaaccaacaggaccctctttatatttggtgtcacaaagtatattgcaggaccctatgaatgtgaaatacggaacccagtgagtgccagccgcagtgacccagtcaccctgaatctcctcccaaagctgtccaagccctacatcacaatcaacaacttaaaccccagagagaataaggatgtcttaaccttcacctgtgaacctaagagtaagaactacacctacatttggtggctaaatggtcagagcctccctgtcagtcccagggtaaagcgacccattgaaaacaggatcctcattctacccaatgtcacgagaaatgaaacaggaccttatcaatgtgaaatacgggaccgatatggtggcatccgcagtgacccagtcaccctgaatgtcctctatggtccagacctccccagcatttacccttcattcacctattaccgttcaggagaaaacctctacttgtcctgcttcgccgagtctaacccacgggcacaatattcttggacaattaatgggaagtttcagctatcaggacaaaagctctctatcccccaaataactacaaagcatagtgggctctatgcttgctctgttcgtaactcagccactggcaaggaaagctccaaatccatcacagtcaaagtctctgactggatattaccctgaattctactagttcctccaattccattttctcccatggaatcacgaagagcaagacccactctgttccagaagccctataagctggaggtggacaactcgatgtaaatttcatgggaaaacccttgtacctgacatgtgagccactcagaactcaccaaaatgttcgacaccataacaacagctactcaaactgtaaaccaggataacaagttgatgacttcacactgtggacagtttttccaaagatgtcagaacaagactccccatcatgataaggctcccacccctcttaaccgtccttgctcatgcctgcctctttcacttggcaggataatgcagtcattagaatttcacatgtagtagcttctgagggtaacaacagagtgtcagatatgtcatctcaacctcaaacttttacgtaacatctcaggggaaatgtggctctctccatcttgcatacagggctcccaatagaaatgaacacagagatattgcctgtgtgtttgcagagaagatggtttctataaagagtaggaaagctgaaattatagtagagtctcctttaaatgcacattgtgtggatggctctcaccatttcctaagagatacagtgtaaaacgtgacagtaatactgattctagcagaataaaacatgtaccacatttgctaa

SEQ ID NO: 5 - DNA, 1640 bp, Homo sapiens
gagaagtgctcctgccctggagagaggctcagcacagaaggaggaaggacagcacagcctacagccgtgctcaggaagtttctggatcctaggctcagctccacagaggagaacacgcaggcgcagagaccatggggcccctctcagcccctccctgcacacagcacatcacctggaaggggctcctgctcacagcatcacttttaaacttctggaacctgcctatcactgctcaagtcacgattgaagccctgccacccaaagtttccgaggggaaggatgttcttctacttgtccacaatttgcctcagaatcttgctggctacatctggtacaaaggacaactgatggacctctaccattacattacatcatatgtagtagacggtcaaataaatatatatgggcctgcatacactggacgagaaacagtatattccaatgcatccctgctgatccagaatgtcacccgggaagacgcaggatcctacaccttacacatcataaagcgaggtgataggactagaggagtaactggatatttcaccttcaacttatacctgaagctgcccaagccctacatcaccatcaacaactcaaaacccagggagaataaggatgtcttagccttcacctgtgaacctaagagtgagaactacacctacatttggtggctaaatggtcagagcctcccggtcagtcccagggtaaagcgacccattgaaaacaggatcctcattctacccagtgtcacgagaaatgaaacaggaccctatgaatgtgaaatacgggaccgagatggtggcatgcgcagtgacccagtcaccctgaatgtcctctatggtccagacctccccagcatttacccttcattcacctattaccgttcaggagaaaacctctacttgtcctgcttcgcggaatctaacccaccggcagagtatttttggacaattaatgggaagtttcagcaatcaggacaaaagctctctatcccccaaattactacaaagcatagagggctctatacttgctctgttcgtaactcagctactggcaaggaaagctccaaatccatgacagtcgaagtctctgctccttcaggaataggacgtcttcctctccttaatccaatatagcagccgtgaagtcatttctgtatttcaggaagactggcagacagttgctttgattcttcctcaaactacttacaatcacctacagtccaaaattgctttttcttcaaggagatttatggaaaagactctgacaaggactcttgaatacaagttcctgataacttcaagatcataccactggactaagaactttcaaaattttaatgaacaggctgataccttcatgaaattctagacaaagaagaaaaaaactccatgttattggactaaataatcaaaagcataatgttttcataattttctatttgaaaatgtgctgattctttgaatgttttattctccagatttatgaactttttttcttgagcaattggtaaagtatacttttgtaaacaaaaattgaaacatttgcttttgctctctatctgagtgccccagaattgggaaactattcatgagtattcatatgtttatggtaataaagttatctgcacaagttca

SEQ ID NO: 6 - DNA, 1448 bp, Homo sapiens
agcacagaaggaggaaggacagcacacctgacagccctgctcaggaagtctctggatcctaggctcatctccacaggggagaacacacagacagcagagaccatgggacccctctcagcccctccctgcactcagcacatcacctggaaggggctcctgctcacagcatcacttttaaacttctggaacctgcccaccactgcccaagtaataattgaagccaagccacccaaagtttccgaggggaaggatgttcttctacttgtccacaatttgccccagaatcttactggctacatctggtacaaagggcaaatgacggacctctaccattacattacatcatatgtagtacacggtcaaattatatatgggcctgcctacagtggacgagaaacagtatattccaatgcatccctgctgatccagaatgtcacacaggaggatgcaggatcctacaccttacacatcataaagcgaggcgatgggactggaggagtaactggatatttcactgtcaccttatactcggagactcccaagccctccatctccagcagcaacttaaaccccagggaggtcatggaggctgtgcgcttaatctgtgatcctgagactccggatgcaagctacctgtggttgctgaatggtcagaacctccctatgactcacaggttgcagctgtccaaaaccaacaggaccctctatctatttggtgtcacaaagtatattgcaggaccctatgaatgtgaaatacggaacccagtgagtgccagccgcagtgacccagtcaccctgaatctcctcccgaagctgcccatgccttacatcaccatcaacaacttaaaccccagggagaagaaggatgtgttagccttcacctgtgaacctaagagtcggaactacacctacatttggtggctaaatggtcagagcctcccggtcagtccgagggtaaagcgacccattgaaaacaggatactcattctacccagtgtcacgagaaatgaaacaggaccctatcaatgtgaaatacgggaccgatatggtggcatccgcagtaacccagtcaccctgaatgtcctctatggtccagacctccccagaatttacccttcattcacctattaccgttcaggagaaaacctcgacttgtcctgctttgcggactctaacccaccggcagagtattcttggacaattaatgggaagtttcagctatcaggacaaaagctctttatcccccaaattactacaaatcatagcgggctctatgcttgctctgttcgtaactcagccactggcaaggaaatctccaaatccatgatagtcaaagtctctgagacagcatctccccaggttacctatgctggtccaaacacctggtttcaagaaatccttctgctgtgacctcccaaagtgctaggattaaaacatgacccaccatg

SEQ ID NO: 7 - DNA, 2067 bp, Homo sapiens
agaagtgctcctgccccgggaagaggctcagtgcagaaggaggaaggacagcacagctgacagccgtgctcaggaagattctggatcctaggctcatctccacagaggagaacacgcagggagcagagaccatggggcccctctcagcccctccctgcacacagcatataacctggaaagggctcctgctcacagcatcacttttaaacttctggaacccgcccaccacagcccaagtcacgattgaagcccagccaccaaaagtttccgaggggaaggatgttcttctacttgtccacaatttgccccagaatcttactggctacatctggtacaaaggacaaatcagggacctctaccattatgttacatcatatatagtagacggtcaaataattaaatatgggcctgcatacagtggacgagaaacagtatattccaatgcatccctgctgatccagaatgtcacccaggaagacacaggatcctacactttacacatcataaagcgaggtgatgggactggaggagtaactggacgtttcaccttcaccttatacctggagactcccaaaccctccatctccagcagcaatttcaaccccagggaggccacggaggctgtgattttaacctgtgatcctgagactccagatgcaagctacctgtggtggatgaatggtcagagcctccctatgactcacagcttgcagctgtctgaaaccaacaggaccctctacctatttggtgtcacaaactatactgcaggaccctatgaatgtgaaatacggaacccagtgagtgccagccgcagtgacccagtcaccctgaatctcctcccgaagctgcccaagccctacatcaccatcaataacttaaaccccagggagaataaggatgtctcaaccttcacctgtgaacctaagagtgagaactacacctacatttggtggctaaatggtcagagcctcccggtcagtcccagggtaaagcgacgcattgaaaacaggatcctcattctacccagtgtcacgagaaatgaaacaggaccctatcaatgtgaaatacgggaccgatatggtggcatccgcagtgacccagtcaccctgaatgtcctctatggtccagacctccccagaatttacccttcattcacctattaccattcaggacaaaacctctacttgtcctgctttgcggactctaacccaccggcacagtattcttggacaattaatgggaagtttcagctatcaggacaaaagctttctatcccccagattactacaaagcatagcgggctctatgcttgctctgttcgtaactcagccactggcaaggaaagctccaaatccgtgacagtcagagtctctgactggacattaccctgaattctactagttcctccaattccatcttctcccatggaacctcaaagagcaagacccactctgttccagaagccctataagtcagagttggacaactcaatgtaaatttcatgggaaaatccttgtacctgatgtctgagccactcagaactcaccaaaatgttcaacaccataacaacagctgctcaaactgtaaacaaggaaaacaagttgatgacttcacactgtggacagcttttcccaagatgtcagaataagactccccatcatgatgaggctctcacccctcttagctgtccttgcttgtgcctgcctctttcacttggcaggataatgcagtcattagaatttcacatgtagtataggagcttctgagggtaacaacagagtgtcagatatgtcatctcaacctcagacttttacataacatctcaggaggaaatgtggctctctccatcttgcatacagggctcccaatagaaatgaacacagagatattgcctgtgtgtttgcagagaagatggtttctataaagagtaggaaagctgaaattatagtagactcccctttaaatgcacattgtgtggatggctctcaccatttcctaagagatacattgtaaaacgtgacagtaagactgattctagcagaataaaacatgtactacatttgctaa

SEQ ID NO: 8 - DNA, 1440 bp, Homo sapiens
agaaggaggcaggacagcactgctgagagctgtgctcaggaagcttctggatcctaggctcatctccacagaggagaacacacagacagcagagaccatggggctcctctcagcccctccctgcacacagcgcatcacctggaaggggctcctgctcacagcatcacttttaaacttctggaacccacccacgactgcccaagtcacgattgaagcccagccaaccaaagtttctgaggggaaggatgttcttctacttgtccacaatttgccccagaatcttactggctacatctggtacaaagggcaaatcagggacctctaccattacattacatcatatgtagtagacggtcaaataattatatatgggcctgcatacagtggacgagaaacaatatattccaatgcatccctgctgatccagaatgtcacccaggaagacgcaggatcctacaccttacacatcataatgggaggtgatgagaatagaggagtaactggacatttcaccttcaccttatatctggagactcccaagccctccatctccagcagcaaattaaaccccagggaggccatggaggctgtgagcttaacctgtgatcctgagactccggacgcaagctacctgtggtggatgaatggtcagagcctccctatgtctcacaggttgcagttgtctgaaaccaacaggaccctctttctattgggtgtcacaaagtacactgcaggaccctatgaatgtgaaatacggaacccagtgagtgccagccgcagtgacccattcaccctgaatctcctcccgaagctgcccaagccctacatcaccatcaacaacttaaaacccagggagaataaggatgtcttaaacttcacctgtgaacctaagagtgagaactacacctacatttggtggctaaatggtcagagcctcccggtcagtcccagggtaaagcgacccattgaaaacaggatcctcattctacccagtgtcacgagaaatgaaacaggaccctatcaatgtgaaataagggaccaatatggtggcatccgcagttacccagtcaccctgaatgtcctctatggtccagacctccccagaatttacccttcattcacctattaccgttcaggagaagtcctctacttgtcctgttctgcggactctaacccaccggcacagtattcttggacaattaatgggaagtttcagctatcaggacaaaagctctttatcccccaaattactacaaagcatagcgggctctatgcttgctctgttcgtaactcagccactggcaaggaaagctccaaatccatgacagtaaaagtctctggtaagcggatcccagtatccttggcaatagggatttaggtggagtctatctggccttcagggaagagtcaggaaaacatttttattcccagcctgcgtcc

SEQ ID NO: 9 - DNA, 1707 bp, Homo sapiens
acagaaggaggaaggacagcacagctgacagccgtgctcagacagcttctggatcccaggctcatctccacagaggagaacacacaggcagcagagaccatggggcccctcccagccccttcctgcacacagcgcatcacctggaaggggctcctgctcacagcatcacttttaaacttctggaacccgcccaccactgccgaagtcacgattgaagcccagccacccaaagtttctgaggggaaggatgttcttctacttgtccacaatttgccccagaatcttcctggctacttctggtacaaaggggaaatgacggacctctaccattacattatatcgtatatagttgatggtaaaataattatatatgggcctgcatacagtggaagagaaacagtatattccaacgcatccctgctgatccagaatgtcacccggaaggatgcaggaacctacaccttacacatcataaagcgaggtgatgagactagagaagaaattcgacatttcaccttcaccttatacttggagactcccaagccctacatctccagcagcaacttaaaccccagggaggccatggaggctgtgcgcttaatctgtgatcctgagactctggacgcaagctacctatggtggatgaatggtcagagcctccctgtgactcacaggttgcagctgtccaaaaccaacaggaccctctatctatttggtgtcacaaagtatattgcaggaccctatgaatgtgaaatacggaacccagtgagtgccagtcgcagtgacccagtcaccctgaatctcctcccgaagctgcccatcccctacatcaccatcaacaacttaaaccccagggagaataaggatgtcttagccttcacctgtgaacctaagagtgagaactacacctacatttggtggctaaacggtcagagcctccccgtcagtcccggggtaaagcgacccattgaaaacaggatactcattctacccagtgtcacgagaaatgaaacaggaccctatcaatgtgaaatacgggaccgatatggtggcctccgcagtaacccagtcatcctaaatgtcctctatggtccagacctccccagaatttacccttcattcacctattaccgttcaggagaaaacctcgacttgtcctgcttcacggaatctaacccaccggcagagtatttttggacaattaatgggaagtttcagcaatcaggacaaaagctctttatcccccaaattactagaaatcatagcgggctctatgcttgctctgttcataactcagccactggcaaggaaatctccaaatccatgacagtcaaagtctctggtccctgccatggagacctgacagagtctcagtcatgactgcaacaactgagacactgagaaaaagaacaggctgataccttcatgaaattcaagacaaagaagaaaaaaactcaatgttattggactaaataatcaaaaggataatgttttcataattttttattggaaaatgtgctgattctttgaatgttttattctccagatttatgaactttttttcttcagcaattggtaaagtatacttttgtaaacaaaaattgaaatatttgcttttgctgtctatctgaatgccccagaattgtgaaactattcatgagtattcataggtttatggtaataaagttatttgcacatgttccgtaa

SEQ ID NO: 10 - DNA, 1591 bp, Homo sapiens
gggtggatcctaggctcatctccataggggagaacacacatacagcagagaccatgggacccctctcagcccctccctgcactcagcacatcacctggaaggggctcctgctcacagcatcacttttaaacttctggaacctgcccaccactgcccaagtaataattgaagcccagccacccaaagtttctgaggggaaggatgttcttctacttgtccacaatttgccccagaatcttactggctacatctggtacaaagggcaaatgacggacctctaccattacattacatcatatgtagtagacggtcaaattatatatgggcctgcctacagtggacgagaaacagtatattccaatgcatccctgctgatccagaatgtcacacaggaggatgcaggatcctacaccttacacatcataaagcgaggcgatgggactggaggagtaactggatatttcactgtcaccttatactcggagactcccaagcgctccatctccagcagcaacttaaaccccagggaggtcatggaggctgtgcgcttaatctgtgatcctgagactccggatgcaagctacctgtggttgctgaatggtcagaacctccctatgactcacaggttgcagctgtccaaaaccaacaggaccctctatctatttggtgtcacaaagtatattgcagggccctatgaatgtgaaatacggaggggagtgagtgccagccgcagtgacccagtcaccctgaatctcctcccgaagctgcccatgccttacatcaccatcaacaacttaaaccccagggagaagaaggatgtgttagccttcacctgtgaacctaagagtcggaactacacctacatttggtggctaaatggtcagagcctcccggtcagtccgagggtaaagcgacccattgaaaacaggatactcattctacccagtgtcacgagaaatgaaacaggaccctatcaatgtgaaatacgggaccgatatggtggcatccgcagtaacccagtcaccctgaatgtcctctatggtccagacctccccagaatttacccttacttcacctattaccgttcaggagaaaacctcgacttgtcctgctttgcggactctaacccaccggcagagtatttttggacaattaatgggaagtttcagctatcaggacaaaagctctttatcccccaaattactacaaatcatagcgggctctatgcttgctctgttcgtaactcagccactggcaaggaaatctccaaatccatgatagtcaaagtctctggtccctgccatggaaaccagacagagtctcattaatggctgccacaatagagacactgagaaaaagaacaggttgataccttcatgaaattcaagacaaagaagaaaaaggctcaatgttattggactaaataatcaaaaggataatgttttcataatttttattggaaaatgtgctgattcttggaatgttttattctccagatttatgaactttttttcttcagcaattggtaaagtatacttttgtaaacaaaaattgaaacatttgcttttgctctctatctgagtgccccccc

SEQ ID NO: 11 - DNA, 1532 bp, Homo sapiens
agaaggaggaaggacagcacagctgacagccgtgctctggaagcttctggatcctaggctcatctccacagaggagaacatgcacgcagcagagatcatggggcccctctcagcccctccctgcacagagcacatcaaatggaaggggctcctgctcacagcattacttttaaacttctggaacttgcctaccactgcccaagtcatgattgaagcccagccacccaaagtgtccgaggggaaggatgttcttctacttgtccacaatttgccccagaatcttactggctacatctggtacaaagggcaaatcagggacctctaccattacattacatcatatgtagtagacggtcaaataattatatatggaccggcatacagtggacgagaaacagtatattccaatgcatccctgctgatccagaatgtcacccgggaggacgcaggatcctacaccttacacatcataaagcgaggtgatgggactagaggagtaactggatatttcaccttcaccttatacctggagactcccaagccctccatctccagcagcaacttaaaccccagggaggccatggagactgtgatcttaacctgtaatcctgagactccggacgcaagctacctgtggtggatgaatggtcagagcctccctatgactcataggatgcagctgtctgaaaccaacaggaccctctttctatttggtgtcacaaagtatactgcaggaccctatgaatgtgaaatatggaactcagggagtgccagccgcagtgacccagtcaccctgaatctcctccatggtccagacctccccagaattttcccttcagtcacctcttactattcaggagagaacctcgacttgtcctgcttcgcaaactctaacccaccagcacagtattcttggacaattaatgggaagtttcagctatcaggacaaaagctctttatccctcagattactccaaagcataatgggctctatgcttgctctgctcgtaactcagccactggcgaggaaagctccacatccttgacaatcagagtcattgctcctccaggattaggaacttttgctttcaataatccaacgtagcagccgtgatgtcatttttgtatttcaggaagactggcaggagatttatggaaaagactatgaaaaggactcttgaatacaagttcctgataacttcaagatcataccactggactaagaactttcaaaattttgatgaacaggctgataccttcatgaaattcaagacaaagaagaaaagaactccatttcattggactaaataacaaaaggataatgttttcataattttttattggaaaatgtgctgattttttgaatgttttatcctccagatttatgaatttttttcttcagcaattggtaaagtatacttttgtaaacaaaaattgaaacatttgcttttgctctctgagtgccccagaattgggaatctattcatgaatattcatatgtttatggtaataaagttatttgcacaagttta

SEQ ID NO: 12 - DNA, 137 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 13 - DNA, 123 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 14 - DNA, 110 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttttt

SEQ ID NO: 15 - DNA, 102 bp, Artificial Sequence
  Description of Artificial Sequence Synthetic polynucleotide
  modified_base (1)..(20): a, c, t, g, unknown or other
nnnnnnnnnnnnnnnnnnnngttttagagctagaaatagcaagttaaaataaggctagtccgttatcaacttgaaaaagtggcaccgagtcggtgctttttt

SEQ ID NO: 16 - DNA, 88 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcaacttgaaaaagtgttttttt

SEQ ID NO: 17 - DNA, 76 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcatttttttt

(19) United States
(12) Patent Application Publication
(10) Pub. No.: US 2023/0348592 A1
(43) Pub. Date: Nov. 2, 2023

(54) METHODS FOR DE-CLOAKING CANCER FROM THE IMMUNE SYSTEM THROUGH DOWNREGULATION OF CANCER-PRODUCED PREGNANCY SPECIFIC GLYCOPROTEIN

(71) Applicants: Memorial Sloan Kettering Cancer Center, New York, NY (US); Institute for Advanced Study, Princeton, NJ (US); The Research Foundation for The State University of New York, Albany, NY (US)

(72) Inventors: James C. Mathews, New York, NY (US); Saad Nadeem, New York, NY (US); Maryam Pouryahya, New York, NY (US); Zehor Belkhatir, New York, NY (US); Joseph O. Deasy, New York, NY (US); Allen Robert Tannenbaum, New York, NY (US); Arnold Levine, Princeton, NJ (US)

(21) Appl. No.: 17/998,634

(22) PCT Filed: May 10, 2021
(86) PCT No.: PCT/US2021/031628

§ 371 Date: Nov. 11, 2022

Related U.S. Application Data
(60) Provisional application No. 63/023,781, filed on May 12, 2020.

(51) Int. Cl. C07K 16/28; A61K 39/395; A61K 39/00; A61K 35/76; A61K 38/21; A61K 38/20; A61K 38/19; A61P 35/00; C12N 15/115; C12N 15/113

(52) U.S. Cl. CPC C07K 16/2803; A61K 39/3955; A61K 39/0011; A61K 35/76; A61K 39/4611; A61K 39/4613; A61K 39/4631; A61K 38/212; A61K 38/215; A61K 38/217; A61K 38/208; A61K 38/195; A61K 38/2086; A61K 38/2013; A61K 38/191; A61P 35/00; C12N 15/115; C12N 15/1135; C12N 2310/14; C12N 2310/531; C12N 2310/11; C07K 2317/22; C07K 2317/31; C07K 2317/21; C07K 2317/24; C07K 2317/622; C07K 2317/569

(57) ABSTRACT

Disclosed are treatments for cancer that enhance a patient immune systems' ability to detect and attack cancer. The present disclosure provides methods for treating cancer in a subject in need thereof comprising administering to the subject an effective amount of a pregnancy specific glycoprotein (PSG) inhibitor.

### CROSS-REFERENCE TO RELATED APPLICATIONS

This application is a U.S. National Stage Application of PCT/US2021/031628, filed May 10, 2021, which claims the benefit of and priority to U.S. Provisional Patent Application No. 63/023,781, filed May 12, 2020, the entire contents of which are incorporated herein by reference.

### STATEMENT REGARDING FEDERALLY SPONSORED RESEARCH

This invention was made with government support under AG048769 and CA087497 awarded by the National Institutes of Health, and FA9550-17-1-0435 awarded by the Air Force Office of Scientific Research (AFOSR). The government has certain rights in the invention.

### SEQUENCE LISTING

The instant application contains a Sequence Listing which has been submitted electronically in ASCII format and is hereby incorporated by reference in its entirety. Said ASCII copy, created on Nov. 7, 2022, is named 115872-2183_SL.txt and is 28,651 bytes in size.

### TECHNICAL FIELD

The present disclosure relates generally to methods for improving the ability of a subject's immune system to detect and treat cancer comprising administering to the subject an effective amount of a pregnancy specific glycoprotein (PSG) inhibitor.

### BACKGROUND

The following description of the background of the present technology is provided simply as an aid in understanding the present technology and is not admitted to describe or constitute prior art to the present technology.

A major problem in data science is representation of data so that the variables driving key functions can be uncovered and explored. Data analysis with a large number of variables always involves evaluating some kind of similarity between variables. Correlation analysis is widely used to simplify networks of feature variables by reducing redundancies, but makes limited use of the network topology, relying on comparison of direct neighbor variables. The disadvantage is that the comparison between variables is made without reference to other variables that could be essential for identifying related function. Consequently, such data analysis is ill-equipped to provide insights into many biological mechanisms.

Cancers may utilize normal physiological processes to escape immune surveillance and evade the immune system. Accordingly, there is a need for therapeutic agents that are useful for mitigating tumor-induced immunosuppression in cancer patients.

### SUMMARY OF THE PRESENT TECHNOLOGY

In one aspect, the present disclosure provides a method for treating cancer in a subject in need thereof comprising administering to the subject a therapeutically effective amount of at least one pregnancy specific glycoprotein (PSG) inhibitor. In some embodiments, the subject comprises at least one tumor that overexpresses one or more PSG genes. The one or more PSG genes may be selected from the group consisting of PSG1, PSG2, PSG3, PSG4, PSG5, PSG6, PSG7, PSG8, PSG9, PSG10, and PSG11. Additionally or alternatively, in some embodiments, the cancer is breast cancer, lung cancer, uterine cancer, colon cancer, rectal cancer, endometrial cancer, stomach cancer, intestinal cancer, renal cancer, acute lymphoblastic leukemia (ALL), chronic lymphocytic leukemia (CLL), acute myeloid leukemia (AML), and metastases thereof.

Additionally or alternatively, in some embodiments of the methods disclosed herein, the at least one PSG inhibitor specifically inhibits one or more PSG genes selected from the group consisting of PSG1, PSG2, PSG3, PSG4, PSG5, PSG6, PSG7, PSG8, PSG9, PSG10, and PSG11. The at least one PSG inhibitor may an antisense oligonucleotide, a sgRNA, a shRNA, a siRNA, an aptamer, a ribozyme, an antibody agent, or a small molecule inhibitor. In some embodiments, the antibody agent that specifically inhibits one or more PSG genes is a monoclonal antibody, a human antibody, a humanized antibody, a multi-specific antibody, a bispecific antibody, a camelised antibody, a chimeric antibody, a Fab, a F(ab′)2, a Fab′, a scFv, a Fv, a Fd, a dAB, a single domain antibody (e.g., nanobody, single domain camelid antibody), a scFv-Fc, a VNAR fragment, a bispecific T-cell engager (BITE) antibody, a minibody, an antibody drug conjugate, a fusion polypeptide, a disulfide-linked Fv (sdFv), an intrabody, or an anti-idiotypic antibody.

In any and all embodiments of the methods disclosed herein, the at least one PSG inhibitor is administered systemically, topically, intravenously, intramuscularly, intraarterially, intrathecally, intracapsularly, intraorbitally, intradermally, intraperitoneally, transtracheally, subcutaneously, intracerebroventricularly, orally, intratumorally, intraocularly, iontophoretically, or intranasally.

Additionally or alternatively, in some embodiments, the method further comprises administering one or more of chemotherapy, radiation therapy, immunotherapy, anti-cancer nucleic acids or anti-cancer proteins to the subject. In certain embodiments, immunotherapy comprises one or more of immune checkpoint inhibitor therapy, adoptive cell therapy, cytokines, immunomodulators, cancer vaccines, monoclonal antibodies, and oncolytic viruses. Examples of cytokines include, but are not limited to, interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2. The adoptive cell therapy may be Tumor-Infiltrating Lymphocyte (TIL) Therapy, Engineered T Cell Receptor (TCR) Therapy, Chimeric Antigen Receptor (CAR) T Cell Therapy, or Natural Killer (NK) Cell Therapy. Additionally or alternatively, in some embodiments, the immune checkpoint inhibitor therapy comprises an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, or an anti-LAG-3 antibody.

In any and all embodiments of the methods disclosed herein, administration of the at least one PSG inhibitor results in one or more improvements in the subject selected among (a) reduced levels and/or activity of CTLA-4 expressing regulatory T cells, (b) inhibition of tumor cell proliferation and/or tumor metastasis, (c) reduced tumor size, (d) amelioration of cancer symptoms, (e) increased weight gain, (f) extended lifespan, (g) prolonged progression-free survival, and (h) decreased risk of cancer therapy-associated side-effects (e.g., autoimmunity).

In one aspect, the present disclosure provides a method for enhancing anti-tumor immune responses in a subject suffering from cancer comprising (a) detecting an increase in mRNA or polypeptide expression levels of one or more PSG genes in a test sample obtained from the subject relative to that in a reference sample or a predetermined threshold, and (b) administering to the subject an effective amount of immune checkpoint blockade therapy. In another aspect, the present disclosure provides a method for enhancing anti-tumor immune responses in a subject suffering from cancer comprising administering to the subject an effective amount of immune checkpoint blockade therapy, wherein mRNA or polypeptide expression and/or activity levels of one or more PSG genes in a test sample obtained from the subject are elevated compared to that in a reference sample or a predetermined threshold. The one or more PSG genes may be selected from the group consisting of PSG1, PSG2, PSG3, PSG4, PSG5, PSG6, PSG7, PSG8, PSG9, PSG10, and PSG11. The immune checkpoint blockade therapy may comprise an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, or an anti-LAG-3 antibody. Additionally or alternatively, in some embodiments, the cancer is breast cancer, lung cancer, uterine cancer, colon cancer, rectal cancer, endometrial cancer, stomach cancer, intestinal cancer, renal cancer, acute lymphoblastic leukemia (ALL), chronic lymphocytic leukemia (CLL), acute myeloid leukemia (AML), and metastases thereof.

In some embodiments of the methods disclosed herein, the polypeptide expression levels of one or more PSG genes are detected via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry. In certain embodiments of the methods disclosed herein, the mRNA expression levels of one or more PSG genes are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH).

Additionally or alternatively, in some embodiments, the test sample comprises blood, plasma, urine, serum, or tumor tissue. Additionally or alternatively, in certain embodiments, the reference sample is a non-tumor biological sample obtained from the subject suffering from cancer or a biological sample obtained from a healthy control subject.

In any of the preceding embodiments, the method further comprises administering chemotherapy, radiation therapy, immunotherapy, anti-cancer nucleic acids or proteins, or combinations thereof to the subject. In any and all of the embodiments of the methods disclosed herein, the anti-tumor responses comprise one or more of increasing levels and/or cytotoxic activity of CD8+ T cells, reducing T cell exhaustion, and/or reduced levels and/or activity of CTLA-4 expressing regulatory T cells.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a block diagram depicting an embodiment of a network environment comprising a client device in communication with server device. FIG. 1B is a block diagram depicting a cloud computing environment comprising client device in communication with cloud service providers. FIGS. 1C and 1D are block diagrams depicting embodiments of computing devices useful in connection with the methods and systems described herein.

FIG. 2 is a flowchart summarizing the Gaussian mixture transport (GMT) analysis pipeline.

FIG. 3 show illustrations of comparison between two features. In this case the two features (VEGFA and PIK3C2A gene expression levels) are not close because of correlation or a direct relationship between their values, but instead because of small GMT distance incorporating the relationships with other features. This comparison metric is an average of a Gaussian Mixture Model and Optimal Mass Transport based metric between the joint distributions (scatter plots) that are relevant according to the network topology. The gene network is the PANTHER (Protein ANalysis THrough Evolutionary Relationships) curated database (http://www.pantherdb.org/), with numerical data synthesized from the network topology using the graph Laplacian as described below.

FIGS. 4A-4D illustrate a special role for Pregnancy-Specific Glycoproteins in cancer, uncovered with the disclosed functional network analysis using the GMT metric. FIG. 4B: A gene network inferred from the TCGA lung adenocarcinoma transcriptome using Pearson correlation cutoffs, analyzed with the GMT method and represented graphically using the GMT distances between genes. The gene coloring reflects the average z-score profile for the third sample cluster identified in the consensus clustering published at the Broad GDAC Firehose (the profiles for the other four of the five GDAC sample clusters did not show clear patterns of expression with respect to the functional network representation). High and low levels are indicated. The graph layout is force-directed. Pearson correlation alone does not account for the grouping of the PSG genes: After removing 4 outliers out of 509, the mean of the absolute value of the Pearson correlation between the expression values for the 11 PSGs was only 0.177, with a SD of 0.201. FIG. 4A: Highlight of the salient group containing highly expressed genes. The group contains all 10 of the Pregnancy-Specific Glycoprotein genes (PSGs), as well as the pseudogene PSG10, and placental genes LGALS13 and HSD3B1. Several of the downregulated genes in the group are closely related to certain Cancer/Testis Antigens, abbreviated CTAs: ADAM2 (ADAMTSL4), NLRP4 (NLRP2), SPATA19 (SPATA31D4), TMPRSS12 (TMPRSS11D), CRISP2 (CRISP1), XAGE3, and SPANXN5. See Almeida et al., Nucleic Acids Res. 37, D816-D819 (2009) for the full list of 276 CTAs. FIG. 4C: Highlight on the second group containing highly expressed genes, including genes similar to the CTAs: SSX4, DPPA2 (DPPA4), CT47A1, CT47A6, ARMC3 (ARMC9), TSPY1 (TSPYL6), RGS22 (RGSL1). FIG. 4D: Kaplan-Meier survival analysis for the TCGA breast, lung adenocarcinoma, uterine corpus endometrial carcinoma, and colon cohorts stratified by the presence of at least one overexpressed PSG. The PSG+ phenotype confers a substantial survival disadvantage in these cancer types. In a few other cancer types, including ovarian cancer, a subset showing the PSG+ phenotype was present but did not confer a statistically significant advantage or disadvantage. Notably, no PSG+ cases were found in the TCGA pancreas cohort.

FIGS. 5A-5G: A synthetic network with K=3 communities containing N=45 nodes total. The network was randomly generated to have inter-community edge connectivity 0.08 out of a possible maximum of 0.68, and intra-community edge connectivity 0.28 out of a possible maximum of 0.32. Random node weightings are generated from the network by the iterated graph Laplacian (F. R. K. Chung, CBMS Regional Conference Series, Conference Board of the Mathematical Sciences, (1997)) applied to an initial node weighting equal to 1 on a randomly selected node and 0 on every other node. 200 node weightings were generated in this manner. Hierarchical clustering was performed with respect to GMT-distance similarity edge scores. FIG. 5A: The original network. FIGS. 5B-5F: 5 selected hierarchical levels from the series. Each node group is labelled by the number of nodes it contains. FIG. 5G: Heatmap showing ordinary hierarchical clustering of the synthesized samples, with the usual rectilinear representation of the hierarchy tree.

FIGS. 6A-6D: Comparison of GMT community detection with greedy optimization of the modularity (Grossman et al., in Proceedings of the 3rd International Workshop on Link Discovery, LinkKDD '05, R. Grossman, Ed. (ACM, New York, NY, 2005), pp. 36-43; Clauset et al., arXiv:cond-mat/0408187 (30 Aug. 2004)), Louvain optimization (Lancichinetti et al., Phys. Rev. 80, 056117 (2009)), and label propagation (Gregory, New J. Physics 12 103018 (2010)), with respect to NMI. The hierarchy-tree graphical representations are shown. PolBlogs and PolBooks are respectively a network of political blog links and a copurchasing network for political books in the United States. Both are provided with manually annotated indications of political leaning for each node (red and blue). For these smaller networks with few, well-defined communities, the established methods outperform GMT. On the larger Cora academic paper citation network, presumably with greater real-world complexity, GMT outperforms the other methods by a factor of 3.7. The visualization substantially reveals the manually annotated subfields, and also seems to suggest an improvement where some putative subfields are divided into multiple distinct groups and closely related to specific alternative subfields. For example, the subfield in red is divided into two tightly clustered groups, one group very close to the subfields in orange and yellow.

FIG. 7A: The GMT hierarchy computed from the PANTHER curated gene network with 2404 nodes and 32113 edges. Approximately 40% of the network is accounted for by well-defined processes. The gene modules appearing here are showing evidence of coordination already at the level of the network topology, without any influence from empirical node weights. This plot and GO term list could be used as a control for a separate data-driven analysis, with new annotations considered significant only when sufficiently distinct from the annotations on this list. FIG. 7B: Scatter plots of the synthesized data for selected nodes/genes, for illustration. The column containing a given gene name represents the ‘behavioral role profile’ of the gene with respect to the other genes. The behavioral role profiles of highly correlated genes are very similar (e.g. the KDR (Kinase Insert Domain Receptor) and VEGFA (Vascular Endothelial Growth Factor A) columns). However, similar functional roles can be observed even for uncorrelated genes. This phenomenon is the key difference between the GMT (Generic Mapping Tools) hierarchy and a standard correlation-based clustering. For example, the functions of VEGFA (Vascular Endothelial Growth Factor A) and PIK3C2A (Phosphatidylinositol-4-Phosphate 3-Kinase Catalytic Subunit Type 2 Alpha) with respect to SRC are similar, even though the scatter plot VEGFA-PIK3C2A shows a high degree of independence. Namely, both stand in a relation of ‘inhibition’ to SRC. At a low level of our hierarchy, VEGFA and PIK3CA and perhaps other SRC inhibitors remain separate. At a middle level of our hierarchy, VEGFA and PIK3CA are closer together. In general mid level clusters may represent clusters of function, while lower levels stratify each function by the specific manner in which the function is carried out.

FIGS. 8A-8D: The GMT hierarchy and tree-assisted Gene Set Enrichment Analysis computed from the RNA expression profiles of the GTEx (Genotype-Tissue Expression) lung tissue samples (left above and below) and breast tissue samples (above right). (Below right) The 18-20 genes of the 50-gene PAM50 breast-tumor prognostic signature which appear in the breast and lung analysis turn out to be exactly the basal-marker subset identified in earlier studies (Mathews et al., NPJ Breast Cancer, 5:30, (2019)). Thus the PAM50 is representative of only one of the key gene modules for breast tissue. Also, a substantial part of the variance in breast tumors captured by the PAM50 is likely due to variance observable already in normal (non-cancer) tissue, and moreover this type of variation is observed in normal tissues other than breast. The analysis provides evidence that the basal-marker gene submodule of the PAM50 is related to the cell cycle machinery including mitosis, chromatin, and the architecture of the mitotic spindle (shown in shades of blue in the two upper plots). It also suggests augmentation of these markers by other genes in the apparent module, including MCM10, HMMR, ASPM, TOP2A, POLQ, RAD54L, and AURKA. Some of these genes are known to be related to breast cancer. For example AURKA was already suggested as part of the simplified 3-gene prognostic signature for breast tumors SCMGENE (Haibe-Kains, C. et al. J National Cancer Inst., 104(4):311-325, (2012)).

FIG. 9A shows the definition of PSG+.

FIG. 9B shows Kaplan-Meier survival analysis for the TCGA lung cancer stratified by the presence of at least one overexpressed PSG.

FIG. 9C shows Kaplan-Meier survival analysis for the TCGA lung cancer stratified by the presence of at least one overexpressed PSG and gender.

FIG. 9D shows Kaplan-Meier survival analysis for the TCGA mesothelioma stratified by the presence of at least one overexpressed PSG.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectorsfor Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

The present disclosure incorporates relational or functional profiles of (not necessarily direct) neighboring variables along multiple common neighbors, which are fitted with Gaussian mixture models and compared using a data metric based on a version of optimal mass transport tailored to Gaussian mixtures. Hierarchical interactive visualization of the result leads to effective unbiased hypothesis generation in biological contexts. The present disclosure provides an unanticipated immunosuppressive mechanism in cancer that resembles maternal-fetal immune tolerance.

A purpose of the disclosure is to improve patient immune systems' ability to detect and attack cancer. This was achieved using novel mathematical methods to understand the systems biology of cancer and uncover a previously poorly understood mechanism whereby cancer cloaks itself with respect to the patient immune system. In many patients, this is accomplished, at least partly, by upregulating one or more pregnancy specific glycoproteins (PSGs). PSGs are a collection of genes and associated proteins that are upregulated during pregnancy in order to “cloak” the embryo from the mother's immune system. PSGs were found to be upregulated in a consistent manner across several cancer types. Various embodiments relate to corresponding cancer treatments. Treatments are aimed at downregulating PSGs through, for example, use of anti-CTLA-4 antibodies.

### Definitions

Unless defined otherwise, all technical and scientific terms used herein generally have the same meaning as commonly understood by one of ordinary skill in the art to which this technology belongs. Generally, the nomenclature used herein and the laboratory procedures in cell culture, molecular genetics, organic chemistry, analytical chemistry and nucleic acid chemistry and hybridization described below are those well-known and commonly employed in the art.

As utilized herein, the terms “approximately,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the terms “exemplary,” “example,” “potential,” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

The indefinite articles “a” and “an,” as used herein in the specification and in the claims, unless clearly indicated to the contrary, should be understood to mean “at least one.” As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), intratumorally, or topically. Administration includes self-administration and the administration by another.

The term “agent” as used herein may refer to a compound or entity of any chemical class including, for example, polypeptides, nucleic acids, carbohydrates, lipids, small molecules, metals, and/or combinations thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. In some particular embodiments, an agent is or comprises a small molecule, an antibody, an antibody fragment, an aptamer, an siRNA, an shRNA, a DNA/RNA hybrid, an antisense oligonucleotide, a ribozyme, a peptide, a peptide mimetic, a peptide nucleic acid (“PNA”) etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety. In some embodiments, an agent is provided and/or utilized in salt form.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complementary sequence can also be an RNA sequence complementary to the DNA sequence or its complementary sequence, and can also be a cDNA.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes solid tumors, blood born tumors, primary and/or metastatic cancers, premalignant, as well as malignant cancers. Examples of cancers include, but are not limited to, neuroblastoma, melanoma, non-Hodgkin's lymphoma, Epstein-Barr related lymphoma, Hodgkin's lymphoma, retinoblastoma, small cell lung cancer, brain tumors, leukemia, epidermoid carcinoma, prostate cancer, renal cell carcinoma, transitional cell carcinoma, breast cancer, ovarian cancer, lung cancer colon cancer, liver cancer, stomach cancer, and other gastrointestinal cancers. In some embodiments, the cancer is a cancer of skin tissues, organs, bone, cartilage, blood and vessels. In certain embodiments, the cancer is a cancer of the head, neck, eye, mouth, throat, esophagus, chest, bone, lung, colon, rectum, stomach, prostate, breast, ovaries, kidney, liver, pancreas and brain.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of cancer. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the term “inhibition therapy” refers to administration of therapy such that level and/or activity of a target gene is reduced (e.g., as compared with that observed under otherwise comparable conditions absent administration of the therapy). In some embodiments, inhibition therapy involves administration of an inhibitor agent. In some embodiments, an inhibitor agent is one whose presence, level, or form may correlate with inhibition (e.g., reduction in level and/or activity) of a target gene, as compared for example with that observed under otherwise comparable conditions absent the inhibitor agent. In some embodiments, an inhibitor agent is a direct inhibitor in that it directly binds to or interacts with a target gene. In some embodiments, an inhibitor is an indirect inhibitor in that it may not bind to or interact with the target itself, but rather may bind to or interact with another entity, with the result that level and/or activity of the target is reduced. To give but a few examples, where a target is or comprises a polypeptide, an inhibitor agent may, for example, bind to the polypeptide (e.g., so that interaction with a binding partner is inhibited), may bind to an interaction partner of the polypeptide (e.g., such that interaction is inhibited), may bind to a substrate or product of the polypeptide (e.g., so that frequency or extent of a reaction is inhibited), may bind to a regulator of the polypeptide (e.g., so that inhibition by the regulator is enhanced or activation by the regulator is reduced), may bind to a nucleic acid encoding the polypeptide (e.g., so that its expression is reduced), or to an agent that directs or impacts expression or processing thereof, etc. In general, an inhibitor agent may be of any chemical class (e.g., may be or comprise a carbohydrate, an isotope, a lipid, a metal, a nucleic acid, a polypeptide, a small molecule, etc.), and/or in some instances may be or comprise a virus or cell.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. One or more bases of the oligonucleotide may also be modified to include a phosphorothioate bond (e.g., one of the two oxygen atoms in the phosphate backbone which is not involved in the internucleotide bridge, is replaced by a sulfur atom) to increase resistance to nuclease degradation. The exact size of the oligonucleotide will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

“Pregnancy specific glycoprotein inhibitor” or “PSG inhibitor”, as used herein, refer to any substance that inhibits or reduces the expression and/or activity of a PSG. In some embodiments, a PSG inhibitor is a substance that inhibits the transcription, expression, binding, activity or stability of a PSG and/or a nucleic acid encoding such a PSG.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “sample” refers to clinical samples obtained from a subject. Biological samples may include tissues, cells, protein or membrane extracts of cells, mucus, sputum, bone marrow, bronchial alveolar lavage (BAL), bronchial wash (BW), and biological fluids (e.g., ascites fluid or cerebrospinal fluid (CSF)) isolated from a subject, as well as tissues, cells and fluids (blood, plasma, saliva, urine, serum etc.) present within a subject.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The term “specific” as used herein in reference to an oligonucleotide means that the nucleotide sequence of the oligonucleotide has at least 12 bases of sequence identity with a portion of a target nucleic acid when the oligonucleotide and the target nucleic acid are aligned. An oligonucleotide that is specific for a target nucleic acid is one that, under the stringent hybridization or washing conditions, is capable of hybridizing to the target nucleic acid of interest and not substantially hybridizing to nucleic acids which are not of interest. Higher levels of sequence identity are desirable and include at least 75%, at least 80%, at least 85%, at least 90%, at least 95% or at least 98% sequence identity.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “subject,” “individual,” or “patient” are used interchangeably and refer to an individual organism, a vertebrate, a mammal, or a human. In certain embodiments, the individual, patient or subject is a human.

As used herein, the terms “target sequence” and “target nucleic acid sequence” refer to a specific nucleic acid sequence to be modulated (e.g., inhibited or downregulated).

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### Pregnancy-Specific Glycoproteins (PSGs)

Pregnancy-specific glycoproteins are normally synthesized by the trophoblasts and excreted into the maternal circulation from the time of trophoblast differentiation until term. PSGs belong to the carcinoembryonic antigen (CEA) family, but are secreted rather than membrane bound. Coding for PSGs is only observed in species in which fetal cells are in direct contact with the maternal circulation, posing a risk of immune rejection by the mother. There are ten PSG genes (numbered 1-9 and 11) and one pseudogene (PSG10) which are localized on a contiguous piece of DNA on chromosome 13.1-13.3. Their DNA sequences are related and so the transcription of one gene may cross hybridize to other PSG genes. Because of this it has been difficult to know if one or more of the PSG genes function in the same or similar ways.

The PSG proteins are produced in small amounts shortly after fertilization and as the placenta forms these glycoproteins are made and secreted by the trophoblasts whose origin is the embryo. The concentration of the PSGs in the blood stream increases to a maximum by the third trimester. The available evidence is that at least some of the PSGs are involved in immunosuppression of the mother's CD-8 T cells preventing a rejection of the fetus because of the allo-antigens expressed by the fetus (Grossman et al., Proceedings of the 3rd International Workshop on Link Discovery, LinkKDD '05, R. Grossman, Ed. (ACM, New York, NY, 2005), pp. 36-43). The PSGs act upon monocytes resulting in the secretion of TGF-3, IL-10 and IL-6. The TGF-β and IL-10 induce FOX-P3 positive T-reg cells, which help to mediate immunosuppression during pregnancy (Jones et al., PloS One 11, e0158050 (2016)). In mice that are pregnant the administration of antibodies directed against two PSGs, alpha-2 and beta-1, result in spontaneous abortion of the embryos (J. Hau et al., Biomed. Biochim. Acta 44, 1255-1259 (1985)).

### Computing and Network Environment

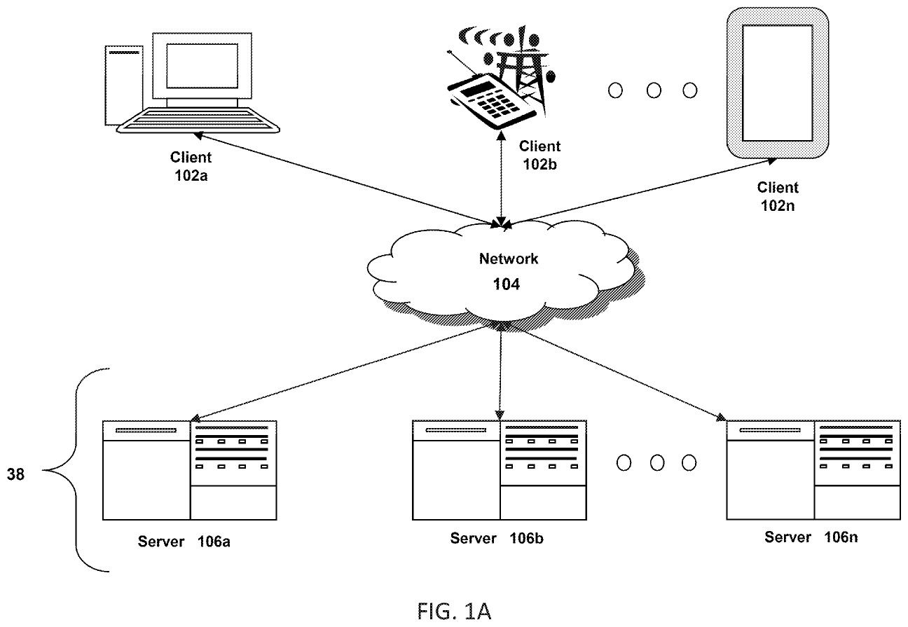

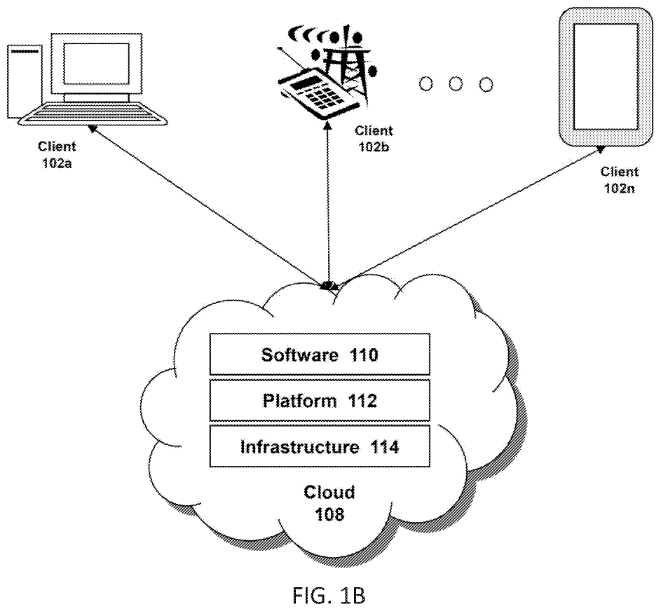

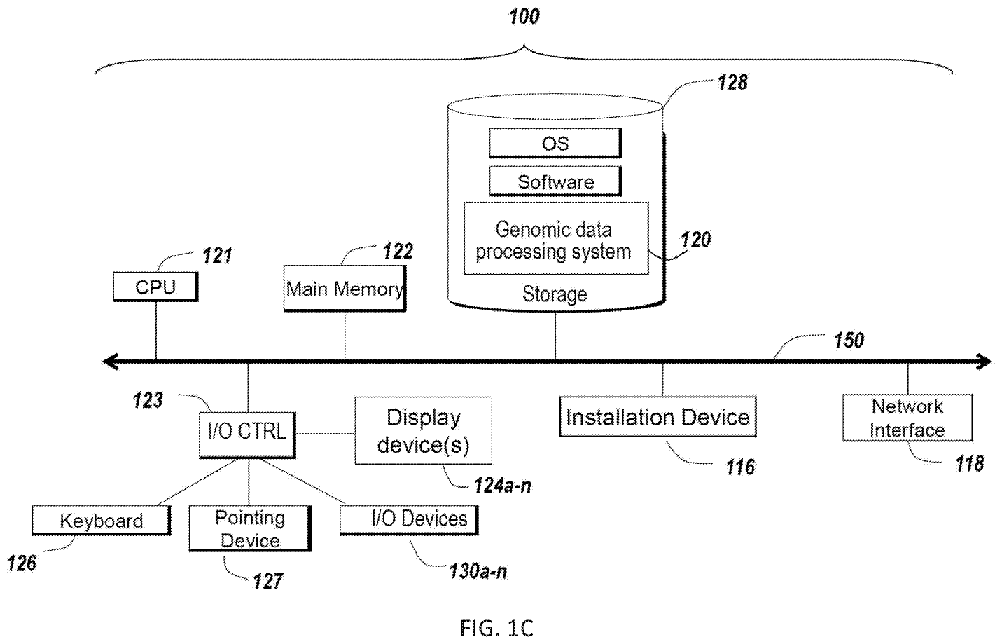

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a genomic data processing system 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DTRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software for the genomic data processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, Ti, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. The computer system 100 can be of any suitable size, such as a standard desktop computer or a Raspberry Pi 4 manufactured by Raspberry Pi Foundation, of Cambridge, United Kingdom. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

Various potential embodiments utilize or otherwise relate to a technique to construct a simplification of a feature network which can be used for interactive data exploration, biological hypothesis generation, and the detection of communities or modules of cofunctional features. These are modules of features that are not necessarily correlated, but nevertheless exhibit common function in their network context as measured by similarity of relationships with neighboring features. In the case of genetic networks, traditional pathway analyses tend to assume that, ideally, all genes in a module exhibit very similar function, independent of relationships with other genes. Potential embodiments of the disclosed technique explicitly relaxes this assumption by employing the comparison of relational profiles. For example, two genes which always activate a third gene are grouped together even if they never do so concurrently. They have common, but not identical, function. The comparison may be driven by an average of a certain computationally-efficient comparison metric between Gaussian Mixture Models, the Gaussian Mixture Transport distance. In various embodiments, the disclosed method has its basis in the local connection structure of the network and the collection of joint distributions of the data associated with nodal neighborhoods. It is benchmarked on networks with known community structures.

In an example implementation, the gene regulatory network in lung adenocarcinoma was analyzed, finding a cofunctional module of genes including the Pregnancy-Specific Glycoproteins (PSGs) and several other placental genes. About 20% of lung, breast, uterus, and colon cancer patients in the TCGA (The Cancer Genome Atlas) have an elevated PSG+ signature, with an associated poor group prognosis. In conjunction with previous results relating PSGs to tolerance in the immune system, these findings implicate the PSGs in a potential immune tolerance mechanism of cancers.

### Therapeutic Methods of the Present Technology

The following discussion is presented by way of example only, and is not intended to be limiting.

One aspect of the present technology includes methods of treating a disease or condition characterized by elevated expression levels and/or increased activity of one or more PSG genes. Additionally or alternatively, in some embodiments, the present technology includes methods of treating cancer. In one aspect, the present disclosure provides a method for treating cancer in a subject in need thereof, comprising administering to the subject a therapeutically effective amount of at least one PSG inhibitor. In some embodiments, the subject exhibits or comprises tumors characterized by elevated expression levels and/or increased activity of one or more PSG genes. The one or more PSG genes may be selected from the group consisting of PSG1, PSG2, PSG3, PSG4, PSG5, PSG6, PSG7, PSG8, PSG9, PSG10, and PSG11. For example, by preventing transcription, repressing mRNA transcript levels, inhibiting translation, or otherwise directly or indirectly interfering with the activity of one or more PSG genes, it is possible to treat and/or inhibit tumor cells, e.g., tumor cells having an increased level of activity and/or expression of one or more PSG genes relative to control. PSG gene expression or activity may be modulated either directly through interaction with a PSG gene, mRNA or protein itself, or indirectly through one or more upstream regulator or downstream targets of one or more PSG genes.

Any PSG inhibitor can be used in the methods of the disclosure, including antisense oligonucleotides, sgRNAs, shRNAs, siRNAs, aptamers, ribozymes, antibody agents, and small molecule inhibitors. In some embodiments of the methods disclosed herein, the PSG inhibitor specifically inhibits one or more PSG genes selected from the group consisting of PSG1, PSG2, PSG3, PSG4, PSG5, PSG6, PSG7, PSG8, PSG9, PSG10, and PSG11. In some embodiments, the antibody agent that specifically inhibits one or more PSG genes is a monoclonal antibody, a human antibody, a humanized antibody, a multi-specific antibody, a bispecific antibody, a camelised antibody, a chimeric antibody, a Fab, a F(ab′)2, a Fab′, a scFv, a Fv, a Fd, a dAB, a single domain antibody (e.g., nanobody, single domain camelid antibody), a scFv-Fc, a VNAR fragment, a bispecific T-cell engager (BITE) antibody, a minibody, an antibody drug conjugate, a fusion polypeptide, a disulfide-linked Fv (sdFv), an intrabody, or an anti-idiotypic antibody.

In one aspect, the present disclosure provides methods of prolonging time to disease progression of cancer (e.g., progression-free survival) in a subject, comprising administering to the subject an effective amount of a PSG inhibitor described herein. In some embodiments, administration of a PSG inhibitor prolongs the time to disease progression by at least 1, 2, 3, 4, 5, 6, 7, 8, 9, 10, 11, 12 weeks. In certain embodiments, administration of a PSG inhibitor prolongs the time to disease progression by at least 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 13, 14, 15, 16, 17, 18, 19, 20, 21, 22, 23, 24, 30, 36, 42, 48, 54, 60, 66, or 72 months.

In another aspect, the present disclosure provides methods of prolonging overall survival of a subject having cancer comprising administering to the subject an effective amount of a PSG inhibitor described herein. In some embodiments, administration of a PSG inhibitor prolongs the survival of the subject by at least 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10.0, 10.2, 10.4, 10.6, 10.8, 11.0, 11.2, 11.4, 11.6, 11.8, 12.0, 13, 14, 15, 16, 17, 18, 19, 20, 21, 22, 23, 24, 30, 36, 42, 48, 54, 60, 66, or 72 months.

In one aspect, the present disclosure provides methods of improving one or more clinical benefits of a subject having cancer comprising administering to the subject an effective amount of a PSG inhibitor described herein. Clinical benefits include, but are not limited to, improved/better quality of life, improved/better symptom control of cancer, inhibition of tumor cell proliferation, inhibition of tumor metastasis, reduction of tumor size, and increased weight gain.

In any and all embodiments of the methods disclosed herein, the methods further comprise administering chemotherapy, radiation therapy, immunotherapy, monoclonal antibodies, anti-cancer nucleic acids or proteins, or combinations thereof to the subject. The immunotherapy may comprise one or more of immune checkpoint inhibitor therapy, adoptive cell therapy (e.g., Tumor-Infiltrating Lymphocyte (TIL) Therapy, Engineered T Cell Receptor (TCR) Therapy, Chimeric Antigen Receptor (CAR) T Cell Therapy, Natural Killer (NK) Cell Therapy), cytokines, immunomodulators, cancer vaccines, monoclonal antibodies, and oncolytic viruses. In some embodiments, the cytokine is selected from the group consisting of interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

In one aspect, the present disclosure provides a method for enhancing anti-tumor immune responses in a subject suffering from cancer comprising (a) detecting an increase in mRNA or polypeptide expression levels of one or more PSG genes in a test sample obtained from the subject relative to that in a reference sample or a predetermined threshold, and (b) administering to the subject an effective amount of immune checkpoint blockade therapy. In another aspect, the present disclosure provides a method for enhancing anti-tumor immune responses in a subject suffering from cancer comprising administering to the subject an effective amount of immune checkpoint blockade therapy, wherein mRNA or polypeptide expression and/or activity levels of one or more PSG genes in a test sample obtained from the subject are elevated compared to that in a reference sample or a predetermined threshold. The one or more PSG genes may be selected from the group consisting of PSG1, PSG2, PSG3, PSG4, PSG5, PSG6, PSG7, PSG8, PSG9, PSG10, and PSG11. Accordingly, in some embodiments, a method is provided for determining the likelihood that a tumor can be effectively treated with an immune checkpoint inhibitor. The immune checkpoint blockade therapy may comprise an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, or an anti-LAG-3 antibody. Additionally or alternatively, in some embodiments, the cancer is breast cancer, lung cancer, uterine cancer, colon cancer, rectal cancer, endometrial cancer, stomach cancer, intestinal cancer, renal cancer, acute lymphoblastic leukemia (ALL), chronic lymphocytic leukemia (CLL), acute myeloid leukemia (AMIL), and metastases thereof.

Additionally or alternatively, in some embodiments, the method further comprises administering chemotherapy, radiation therapy, immunotherapy, monoclonal antibodies, anti-cancer nucleic acids or proteins, or combinations thereof to the subject. The immunotherapy may comprise one or more of adoptive cell therapy (e.g., Tumor-Infiltrating Lymphocyte (TIL) Therapy, Engineered T Cell Receptor (TCR) Therapy, Chimeric Antigen Receptor (CAR) T Cell Therapy, Natural Killer (NK) Cell Therapy), cytokines, immunomodulators, cancer vaccines, monoclonal antibodies, and oncolytic viruses. In some embodiments, the cytokine is selected from the group consisting of interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2. The test sample may be tissues, cells, biological fluids (blood, plasma, saliva, urine, serum etc.) or tumors present within a subject. In some embodiments, the reference sample is a non-tumor cell, non-tumor tissue, or non-tumor biological sample obtained from the subject suffering from cancer or from a healthy control subject. Generally, the methods include a step of detecting the expression and/or activity of one or more PSG genes. Gene products can be detected in a test sample and can be polypeptides or polynucleotides, e.g., mRNA.

In any and all of the embodiments of the methods disclosed herein, the anti-tumor responses comprise one or more of increasing levels and/or cytotoxic activity of CD8+ T cells, reducing T cell exhaustion, and/or reduced levels and/or activity of CTLA-4 expressing regulatory T cells.

Nucleic acid analysis. Detection of one or more PSG genes at the nucleic acid level is encompassed by embodiments of the present disclosure. Nucleic acid analyses can be performed on messenger RNAs, and/or cDNA. In many embodiments, nucleic acids are extracted from a biological sample, e.g. a tumor sample. In some embodiments, nucleic acids are analyzed without having been amplified. In some embodiments, nucleic acids are amplified using techniques known in the art (such as polymerase chain reaction (PCR)) and amplified nucleic acids are used in subsequent analyses. Multiplex PCR, in which several amplicons (e.g., from different genomic regions) are amplified at once using multiple sets of primer pairs, may be employed. (see generally, Bustin, S. A., 2000 J. Molecular Endocrinology 25:169-193.)

Additional techniques include, but are not limited to, in situ hybridization, Northern blot, and various nucleic acid amplification techniques such as PCR, RT-PCR, quantitative RT-PCR, and the ligase chain reaction. PCR and considerations for primer design are well known in the art and are described, for example, in Newton, et al. (eds.) PCR: Essential data Series, John Wiley & Sons; PCR Primer: A Laboratory Manual, Cold Spring Harbor Laboratory Press, Cold Spring Harbor, N Y, 1995; White, et al. (eds.) PCR Protocols: Current methods and Applications, Methods in Molecular Biology, The Humana Press, Totowa, N J, 1993. In certain embodiments of the methods disclosed herein, the mRNA expression levels of one or more PSG genes are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH).

Polypeptide analysis. Polypeptide levels of one or more PSG genes can be detected using any of a variety of techniques and binding agents. In certain embodiments, a binding agent is an antibody that binds specifically to one or more PSG genes. The disclosure also encompasses the use of protein arrays, including antibody arrays, for detection of polypeptide levels of one or more PSG genes. The use of antibody arrays is described, for example, in Haab et al., Genome Biol. 2(2):2001 (2001). Other types of protein arrays known in the art are useful in the disclosed methods. In general, antibodies that bind specifically to polypeptides of one or more PSG genes can be generated by methods well known in the art and described, for example, in Harlow, E, Lane, E, and Harlow, E, (eds.) Using Antibodies: A Laboratory Manual, Cold Spring Harbor Laboratory Press, Cold Spring Harbor, 1998. Details and references for the production of antibodies may also be found in U.S. Pat. No. 6,008,337. Antibodies may include, but are not limited to, polyclonal, monoclonal, chimeric (e.g., “humanized”), single chain antibodies, Fab fragments, antibodies generated using phage display technology, etc. The present technology encompasses the use of “fully human” antibodies produced using the XenoMouse™ technology (AbGenix Corp., Fremont, CA) according to the techniques described in U.S. Pat. No. 6,075,181. Antibody detection methods are well known in the art including, but are not limited to, enzyme-linked immunosorbent assays (ELISAs) and Western blots. Some such methods are amenable to being performed in an array format.

In addition, in certain embodiments of the disclosure, polypeptides levels of one or more PSG genes are detected using other specific binding agents known in the art for the detection of polypeptides, such as aptamers (Aptamers, Molecular Diagnosis, Vol. 4, No. 4, 1999), reagents derived from combinatorial libraries for specific detection of proteins in complex mixtures, random peptide affinity reagents, etc. In general, any appropriate binding agent for detecting a polypeptide may be used in conjunction with the present technology, although antibodies may represent a particularly appropriate modality.

In certain embodiments of the disclosure, a single binding agent (e.g., antibody) is used whereas in other embodiments of the present technology multiple binding agents, directed either against the same or against different PSG polypeptides can be used to increase the sensitivity or specificity of the detection technique or to provide more detailed information than that provided by a single binding agent. Thus the disclosure encompasses the use of a battery of binding agents that bind to polypeptides encoded by one or more PSG genes described herein. In general, polypeptides may be detected within a tumor sample that has been obtained from a subject, e.g., a tissue sample, cell sample, cell extract, body fluid sample, etc.

In certain embodiments, binding can be detected by adding a detectable label to a binding agent. In other embodiments, binding can be detected by using a labeled secondary binding agent that associates specifically with a primary binding agent, e.g., as is well known in the art of antigen/antibody detection. A detectable label may be directly detectable or indirectly detectable, e.g., through combined action with one or more additional members of a signal producing system. Examples of directly detectable labels include radioactive, paramagnetic, fluorescent, light scattering, absorptive and colorimetric labels. Indirectly detectable labels include chemiluminescent labels, e.g., enzymes that are capable of converting a substrate to a chromogenic product such as alkaline phosphatase, horseradish peroxidase and the like.

Once a labeled binding agent has bound a polypeptide of one or more PSG genes, the complex may be visualized or detected in a variety of ways, with the particular manner of detection being chosen based on the particular detectable label. Representative detection means include, e.g., scintillation counting, autoradiography, measurement of paramagnetism, fluorescence measurement, light absorption measurement, measurement of light scattering and the like. Depending upon the nature of the sample, appropriate detection techniques include, but are not limited to, immunohistochemistry (IHC), radioimmunoassay, ELISA, immunoblotting and fluorescence activated cell sorting (FACS). In the case where the polypeptide is to be detected in a tissue sample, e.g., a biopsy sample, IHC is a particularly appropriate detection technique.

In certain embodiments, detection techniques of the present disclosure include a negative control, which can involve applying assaying a control sample (e.g., from a normal non-cancerous tissue) so that a signal obtained thereby can be compared with a signal obtained from a tumor sample being tested. In tests in which a secondary binding agent is used to detect a primary binding agent that binds to the polypeptide of interest, an appropriate negative control can involve performing the test on a portion of the sample with the omission of the primary binding agent.

In general, results of detection techniques can be presented in any of a variety of formats. The results can be presented in a qualitative fashion. For example, a test report may indicate only whether or not a particular PSG polypeptide was detected, perhaps also with an indication of the limits of detection. Results may be presented in a semi-quantitative fashion. For example, various ranges may be defined, and the ranges may be assigned a score (e.g., 0 to 3 where 0 means no binding detected and 3 means strong binding detected) that provides a certain degree of quantitative information. Such a score may reflect various factors, e.g., the number of cells in which a polypeptide is detected, the intensity of the signal (which may indicate the level of expression of a polypeptide), etc. The results may be presented in a quantitative fashion, e.g., as a percentage of cells in which a polypeptide is detected, as a protein concentration, etc.

In some embodiments of the methods disclosed herein, the polypeptide expression levels of one or more PSG genes are detected via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry.

Detectable moieties. In certain embodiments, certain molecules (e.g., nucleic acid probes, antibodies, etc.) used in accordance with and/or provided by the disclosure comprise one or more detectable entities or moieties, i.e., such molecules are“labeled” with such entities or moieties. Nonlimiting examples of detectable moieties include, without limitation, fluorescent compounds, various enzymes, prosthetic groups, luminescent materials, bioluminescent materials, fluorescent emitting metal atoms, (e.g., europium (Eu)), radioactive isotopes (described below), quantum dots, electron-dense reagents, and haptens. The detectable moieties can be detected using various means including, but are not limited to, spectroscopic, photochemical, radiochemical, biochemical, immunochemical, or chemical means.

A detectable moiety can also be a detectable enzyme, such as alkaline phosphatase, horseradish peroxidase, beta-galactosidase, acetylcholinesterase, glucose oxidase and the like. When a nanopolymer is derivatized with a detectable enzyme, it can be detected by adding additional reagents that the enzyme uses to produce a detectable reaction product. For example, when the detectable moiety is horseradish peroxidase, the addition of hydrogen peroxide and diaminobenzidine leads to a detectable colored reaction product. A polypeptide can also be derivatized with a prosthetic group (e.g., streptavidin/biotin and avidin/biotin). For example, a polypeptide can be derivatized with biotin and detected through indirect measurement of avidin or streptavidin binding. Nonlimiting examples of fluorescent compounds that can be used as detectable moieties include 5-dimethylamine-1-napthalenesulfonyl chloride, umbelliferone, fluorescein, fluorescein isothiocyanate, rhodamine, dichlorotriazinylamine fluorescein, dansyl chloride, and phycoerythrin. Luminescent materials include, e.g., luminol, and bioluminescent materials include, e.g., luciferase, luciferin, and aequorin.

A detectable moiety can also be a radioactive isotope, such as, but not limited to, alpha-, beta-, or gamma-emitters; or beta- and gamma-emitters. Radioactive isotopes can be used in diagnostic or therapeutic applications. Such radioactive isotopes include, but are not limited to, iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium (¹¹¹In), technetium (^{99m}Tc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and gallium (⁶⁷Ga).

Various PSG inhibitors are known and/or commercially available. Exemplary PSG inhibitors, are described below.

### PSG-Specific Inhibitory Nucleic Acids

Exemplary mRNA sequences of the eleven human PSG genes, represented by SEQ ID NOs: 1-11, are provided below:

Homo sapiens pregnancy specific beta-1-glycoprotein 1 (PSG1), transcript variant 4, mRNA
(NCBI Reference Sequence: NM_001297773.2)
(SEQ ID NO: 1)
GAGAATTGCTCCTGCCCTGGGAAGAGGCTCAGCACAGAAAGAGGAAGGACAGCACAGCTGACAGCCGTGC
TCAGAGAGTTTCTGGATCCTAGGCTTATCTCCACAGAGGAGAACACACAAGCAGCAGAGACCATGGGAAC
CCTCTCAGCCCCTCCCTGCACACAGCGCATCAAATGGAAGGGGCTCCTGCTCACAGCATCACTTTTAAAC
TTCTGGAACCTGCCCACCACTGCCCAAGTCACGATTGAAGCCGAGCCAACCAAAGTTTCCGAGGGGAAGG
ATGTTCTTCTACTTGTCCACAATTTGCCCCAGAATCTTACCGGCTACATCTGGTACAAAGGGCAAATGAG
GGACCTCTACCATTACATTACATCATATGTAGTAGACGGTGAAATAATTATATATGGGCCTGCATATAGT
GGACGAGAAACAGCATATTCCAATGCATCCCTGCTGATCCAGAATGTCACCCGGGAGGACGCAGGATCCT
ACACCTTACACATCATAAAGGGAGATGATGGGACTAGAGGAGTAACTGGACGTTTCACCTTCACCTTACA
CCTGGAGACTCCTAAGCCCTCCATCTCCAGCAGCAACTTAAATCCCAGGGAGACCATGGAGGCTGTGAGC
TTAACCTGTGACCCTGAGACTCCAGACGCAAGCTACCTGTGGTGGATGAATGGTCAGAGCCTCCCTATGA
CTCACAGCTTGAAGCTGTCCGAAACCAACAGGACCCTCTTTCTATTGGGTGTCACAAAGTATACTGCAGG
ACCCTATGAATGTGAAATACGGAACCCAGTGAGTGCCAGCCGCAGTGACCCAGTCACCCTGAATCTCCTC
CCGAAGCTGCCCAAGCCCTACATCACCATCAACAACTTAAACCCCAGGGAGAATAAGGATGTCTTAAACT
TCACCTGTGAACCTAAGAGTGAGAACTACACCTACATTTGGTGGCTAAATGGTCAGAGCCTCCCGGTCAG
TCCCAGGGTAAAGCGACCCATTGAAAACAGGATCCTCATTCTACCCAGTGTCACGAGAAATGAAACAGGA
CCCTATCAATGTGAAATACGGGACCGATATGGTGGCATCCGCAGTGACCCAGTCACCCTGAATGTCCTCT
ATGGTCCAGACCTCCCCAGAATTTACCCTTCATTCACCTATTACCGTTCAGGAGAAGTCCTCTACTTGTC
CTGTTCTGCGGACTCTAACCCACCGGCACAGTATTCTTGGACAATTAATGAAAAGTTTCAGCTACCAGGA
CAAAAGCTCTTTATCCGCCATATTACTACAAAGCATAGCGGGCTCTATGTTTGCTCTGTTCGTAACTCAG
CCACTGGCAAGGAAAGCTCCAAATCCATGACAGTCGAAGTCTCTGCTTATAGCAGTTCAATAAACTATAC
TTCTGGGAACCGTAATTGAAACATTTACTTTTGCTTTCTACCTGACTGCCCCAGAATTGGGCAACTATTC
ATGAGAATTGATATGTTTATGGTAATACACATATTTGCACAAGTACAGTAACAATCTGCTTTCTTTGTAA
CATGACACATTTGAAATCATTGGTTATATTACCAATGCTTTGATTCGGATGTTATATTAAAAACATAGAT
AGAATGAACCAATATGAACTGCAGGCAAAGTCTGAAGTCAGCCTTGGTTTGGCTTCCTATTCTCAAGAGG
TTTGTGAAGATTTAATCTCAGATTCCTTATAAAAACTTAGAGAAAAGAAAATTTTAGAAGACAGCCTACA
TGGTCCATTGCTACTCTTGCTGCACTTATGTAAACAATCAGACCACATTTGAAGAAACTCCACCTATTTT
GCAAACAAACTTATTCTACTGAAATTATCATTGGTAAAAGTAGAGATGCCCATAGAGGGAAAAATTATGT
GGAAAATAAAAACTGTAGTATACCTGT
Homo sapiens pregnancy specific beta-1-glycoprotein 2 (PSG2), mRNA (NCBI Reference
Sequence: NM_031246.4)
(SEQ ID NO: 2)
GGGAATTGCTGCTGCCCCAGGAAGAGGCTCAGTGCAGAAGGAGGAAGGACAGCACAGCTGACAGCCGTGC
TCAGGAAGTTTCTGGATCCTAGGCTCATCTCCACAGAGGAGAACACACAGGCAGCAGAGACCATGGGGCC
CCTCTCAGCCCCTCCCTGCACAGAGCACATCAAATGGAAGGGGCTCCTGGTCACAGCATCACTTTTAAAC
TTCTGGAACCTGCCCACCACTGCCCAAGTCACGATTGAAGCCCAGCCACCAAAAGTTTCCGAGGGGAAGG
ATGTTCTTCTACTTGTCCACAATTTGCCCCAGAATCTTACTGGCTACATCTGGTACAAAGGGCAAATCAG
GGACCTCTACCATTACATTACATCATATGTAGTAGACGGTCAAATAATTATATATGGGCCTGCATATAGT
GGACGAGAAACAGCATATTCCAATGCATCCCTGCTGATCCAGAATGTCACCCGGGAGGACGCAGGATCCT
ACACCTTACACATCATAAAGCGAGGTGATGGGACTAGAGGAGTAACTGGATATTTCACCTTCACCTTATA
CCTGGAGACTCCCAAGCCCTCCATCTCCAGCAGCAACTTAAACCCCAGGGAGGCCATGGAAACTGTGATC
TTAACCTGTGATCCTGAGACTCCGGACACAAGCTACCAGTGGTGGATGAATGGTCAGAGCCTCCCTATGA
CTCATAGGTTTCAGCTGTCCGAAACCAACAGGACCCTCTTTCTATTTGGTGTCACAAAGTATACTGCAGG
ACCCTATGAATGTGAAATACGGAACTCAGGGAGTGCCAGCCGCAGTGACCCAGTCACCCTGAATCTCCTC
CATGGTCCAGACCTCCCCAGAATTCACCCTTCATACACCAATTACCGTTCAGGAGATAACCTCTACTTGT
CTTGCTTCGCGAACTCTAACCCACCGGCACAGTATTCTTGGACAATTAATGGGAAGTTTCAGCAATCAGG
ACAAAATCTGTTTATCCCCCAAATTACTACAAAGCATAGCGGGCTCTATGTTTGCTCTGTTCGTAACTCA
GCCACTGGCGAGGAAAGCTCCACATCGTTGACAGTCAAAGTCTCTGCTTCTACAAGAATAGGACTTCTTC
CTCTCCTTAATCCAACATAGCAGCTGTGATGTCATTTCTGTATTTCAGGAAGACTGGCAGGAGATTTATG
GAAAGGTCTCTTACAAGGACTCTTGAATACAAGCTCCTGATAACTTCAAGATCATACCACTGGACTAAGA
ACTTTCAAAATTTTAATGAACAGGCTGATACCTTCATGAAATTCAAGACAAAGAAGAAAAATACTCAATG
TTATTGGACTAAATAATCAAAAGGATAATGATTTCATAATTTTCTATTTGAAAATGTGCTGATTCTTGGA
ATGTTTCATTCTCCAGATTTATGAACATTTTTTCTTGAGCAATTGGTAAAGTATACTTTTGTAAACAAAA
ATTGAAACATTTCCTTTTGCTCTCTATCTGAGTGCCCCAGAATTGGGAATCTATTCATGAGTATTCATAT
GTTTATGGTAATAAAGCTATTTGCACAAGTTCA
Homo sapiens pregnancy specific beta-1-glycoprotein 3 (PSG3), mRNA (NCBI Reference
Sequence: NM_021016.4)
(SEQ ID NO: 3)
AGAAGGAGGAAGGACAGCACAGCTGAGAGCCATGCTCAGGAAGTTTCTGGATCCTAGGCTCAGCTCCACA
GAGGAGAACACGCAGGCAGCAGAGACCATGGGGCCCCTCTCAGCCCCTCCCTGCACACAGCGCATCACCT
GGAAGGGGCTCCTGCTCACAGCATTACTTTTAAACTTCTGGAACTTGCCTACCACTGCCCAAGTCACGAT
TGAAGCCGAGCCAACCAAAGTTTCCAAGGGGAAGGACGTTCTTCTACTTGTCCACAATTTGCCCCAGAAT
CTTGCTGGCTACATCTGGTACAAAGGGCAAATGAAGGACCTCTACCATTACATTACATCATACGTAGTAG
ATGGTCAAATAATTATATATGGGCCTGCATACAGTGGACGAGAAACAGTATATTCCAATGCATCCCTGCT
GATCCAGAATGTCACCCGGGAGGACGCAGGATCCTACACCTTACACATCGTAAAGCGAGGTGATGGGACT
AGAGGAGAAACTGGACATTTCACCTTCACCTTATACCTGGAGACTCCCAAGCCCTCCATCTCCAGCAGCA
ACTTATACCCCAGGGAGGACATGGAGGCTGTGAGCTTAACCTGTGATCCTGAGACTCCGGACGCAAGCTA
CCTGTGGTGGATGAATGGTCAGAGCCTCCCTATGACTCACAGCTTGCAGTTGTCCAAAAACAAAAGGACC
CTCTTTCTATTTGGTGTCACAAAGTACACTGCAGGACCCTATGAATGTGAAATACGGAACCCAGTGAGTG
CCAGCCGCAGTGACCCAGTCACCCTGAATCTCCTCCCGAAGCTGCCCAAGCCCTACATCACCATCAACAA
CTTAAACCCCAGGGAGAATAAGGATGTCTTAGCCTTCACCTGTGAACCTAAGAGTGAGAACTACACCTAC
ATTTGGTGGCTAAATGGTCAGAGCCTCCCGGTCAGTCCCAGGGTAAAGCGACCCATTGAAAACAGGATCC
TCATTCTACCCAGTGTCACGAGAAATGAAACAGGACCCTATCAATGTGAAATACAGGACCGATATGGTGG
CATCCGCAGTTACCCAGTCACCCTGAATGTCCTCTATGGTCCAGACCTCCCCAGAATTTACCCTTCATTC
ACCTATTACCATTCAGGAGAAAACCTCTACTTGTCCTGCTTCGCGGACTCTAACCCACCAGCAGAATATT
CTTGGACAATTAATGGGAAGTTTCAGCTATCAGGACAAAAGCTCTTTATCCCCCAGATTACTACAAAGCA
TAGCGGGCTCTATGCTTGCTCTGTTCGTAACTCAGCCACTGGCATGGAAAGCTCCAAATCCATGACAGTC
AAAGTCTCTGCTCCTTCAGGAACAGGACATCTTCCTGGCCTTAATCCATTATAGCAGCCGTGATGTCATT
TCTGTATTTCAGGAAGACTGGCAGACAGTTGCTTTCATTCTTCCTCAAAGTATTTACCATCAGCTACAGT
CCAAAATTGCTTTTTGTTCAAGGAGATTTATGAAAAGACTCTGACAAGGACTCTTGAATACAAGTTCCTG
ATAACTTCAAGATCATACCACTGGACTAAGAACTTTCAAAATTTTAATGAACAGGCTGATACTTCATGAA
ATTCAAGACAAAGAAAAAAACCCAATTTTATTGGACTAAATAGTCAAAACAATGTTTTCATAATTTTCTA
TTTGAAAATGTGCTGATTCTTTGAATGTTTTATTCTCCAGATTTATGCACTTTTTTTCTTCAGCAATTGG
TAAAGTATACTTTTGTAAACAAAAATTGAAACATTTGCTTTTGCTCCCTAAGTGCCCCAGAATTGGGAAA
CTATTCAGGAGTATTCATATGTTTATGGTAATAAAGTTATCTGCACAAGTTCA
Homo sapiens pregnancy specific beta-1-glycoprotein 4 (PSG4), transcript variant 1, mRNA
(NCBI Reference Sequence: NM_002780.5)
(SEQ ID NO: 4)
AGCACAGAAGGAGGAAGGACAGCACAGCTGACAGCCGTACTCAGGAAGCTTCTGGATCCTAGGCTTATCT
CCACAGAGGAGAACACACAAGCAGCAGAGACCATGGGGCCCCTCTCAGCCCCTCCCTGCACACAGCGCAT
CACCTGGAAGGGGGTCCTGCTCACAGCATCACTTTTAAACTTCTGGAATCCGCCCACAACTGCCCAAGTC
ACGATTGAAGCCCAGCCACCCAAAGTTTCTGAGGGGAAGGATGTTCTTCTACTTGTCCACAATTTGCCCC
AGAATCTTGCTGGCTACATTTGGTACAAAGGGCAAATGACATACCTCTACCATTACATTACATCATATGT
AGTAGACGGTCAAAGAATTATATATGGGCCTGCATACAGTGGAAGAGAAAGAGTATATTCCAATGCATCC
CTGCTGATCCAGAATGTCACGCAGGAGGATGCAGGATCCTACACCTTACACATCATAAAGCGACGCGATG
GGACTGGAGGAGTAACTGGACATTTCACCTTCACCTTACACCTGGAGACTCCCAAGCCCTCCATCTCCAG
CAGCAACTTAAATCCCAGGGAGGCCATGGAGGCTGTGATCTTAACCTGTGATCCTGCGACTCCAGCCGCA
AGCTACCAGTGGTGGATGAATGGTCAGAGCCTCCCTATGACTCACAGGTTGCAGCTGTCCAAAACCAACA
GGACCCTCTTTATATTTGGTGTCACAAAGTATATTGCAGGACCCTATGAATGTGAAATACGGAACCCAGT
GAGTGCCAGCCGCAGTGACCCAGTCACCCTGAATCTCCTCCCAAAGCTGTCCAAGCCCTACATCACAATC
AACAACTTAAACCCCAGAGAGAATAAGGATGTCTTAACCTTCACCTGTGAACCTAAGAGTAAGAACTACA
CCTACATTTGGTGGCTAAATGGTCAGAGCCTCCCTGTCAGTCCCAGGGTAAAGCGACCCATTGAAAACAG
GATCCTCATTCTACCCAATGTCACGAGAAATGAAACAGGACCTTATCAATGTGAAATACGGGACCGATAT
GGTGGCATCCGCAGTGACCCAGTCACCCTGAATGTCCTCTATGGTCCAGACCTCCCCAGCATTTACCCTT
CATTCACCTATTACCGTTCAGGAGAAAACCTCTACTTGTCCTGCTTCGCCGAGTCTAACCCACGGGCACA
ATATTCTTGGACAATTAATGGGAAGTTTCAGCTATCAGGACAAAAGCTCTCTATCCCCCAAATAACTACA
AAGCATAGTGGGCTCTATGCTTGCTCTGTTCGTAACTCAGCCACTGGCAAGGAAAGCTCCAAATCCATCA
CAGTCAAAGTCTCTGACTGGATATTACCCTGAATTCTACTAGTTCCTCCAATTCCATTTTCTCCCATGGA
ATCACGAAGAGCAAGACCCACTCTGTTCCAGAAGCCCTATAAGCTGGAGGTGGACAACTCGATGTAAATT
TCATGGGAAAACCCTTGTACCTGACATGTGAGCCACTCAGAACTCACCAAAATGTTCGACACCATAACAA
CAGCTACTCAAACTGTAAACCAGGATAACAAGTTGATGACTTCACACTGTGGACAGTTTTTCCAAAGATG
TCAGAACAAGACTCCCCATCATGATAAGGCTCCCACCCCTCTTAACCGTCCTTGCTCATGCCTGCCTCTT
TCACTTGGCAGGATAATGCAGTCATTAGAATTTCACATGTAGTAGCTTCTGAGGGTAACAACAGAGTGTC
AGATATGTCATCTCAACCTCAAACTTTTACGTAACATCTCAGGGGAAATGTGGCTCTCTCCATCTTGCAT
ACAGGGCTCCCAATAGAAATGAACACAGAGATATTGCCTGTGTGTTTGCAGAGAAGATGGTTTCTATAAA
GAGTAGGAAAGCTGAAATTATAGTAGAGTCTCCTTTAAATGCACATTGTGTGGATGGCTCTCACCATTTC
CTAAGAGATACAGTGTAAAACGTGACAGTAATACTGATTCTAGCAGAATAAAACATGTACCACATTTGCT
AA
Homo sapiens pregnancy specific beta-1-glycoprotein 5 (PSG5), transcript variant 1, mRNA
(NCBI Reference Sequence: NM_002781.4)
(SEQ ID NO: 5)
GAGAAGTGCTCCTGCCCTGGAGAGAGGCTCAGCACAGAAGGAGGAAGGACAGCACAGCCTACAGCCGTGC
TCAGGAAGTTTCTGGATCCTAGGCTCAGCTCCACAGAGGAGAACACGCAGGCGCAGAGACCATGGGGCCC
CTCTCAGCCCCTCCCTGCACACAGCACATCACCTGGAAGGGGCTCCTGCTCACAGCATCACTTTTAAACT
TCTGGAACCTGCCTATCACTGCTCAAGTCACGATTGAAGCCCTGCCACCCAAAGTTTCCGAGGGGAAGGA
TGTTCTTCTACTTGTCCACAATTTGCCTCAGAATCTTGCTGGCTACATCTGGTACAAAGGACAACTGATG
GACCTCTACCATTACATTACATCATATGTAGTAGACGGTCAAATAAATATATATGGGCCTGCATACACTG
GACGAGAAACAGTATATTCCAATGCATCCCTGCTGATCCAGAATGTCACCCGGGAAGACGCAGGATCCTA
CACCTTACACATCATAAAGCGAGGTGATAGGACTAGAGGAGTAACTGGATATTTCACCTTCAACTTATAC
CTGAAGCTGCCCAAGCCCTACATCACCATCAACAACTCAAAACCCAGGGAGAATAAGGATGTCTTAGCCT
TCACCTGTGAACCTAAGAGTGAGAACTACACCTACATTTGGTGGCTAAATGGTCAGAGCCTCCCGGTCAG
TCCCAGGGTAAAGCGACCCATTGAAAACAGGATCCTCATTCTACCCAGTGTCACGAGAAATGAAACAGGA
CCCTATGAATGTGAAATACGGGACCGAGATGGTGGCATGCGCAGTGACCCAGTCACCCTGAATGTCCTCT
ATGGTCCAGACCTCCCCAGCATTTACCCTTCATTCACCTATTACCGTTCAGGAGAAAACCTCTACTTGTC
CTGCTTCGCGGAATCTAACCCACCGGCAGAGTATTTTTGGACAATTAATGGGAAGTTTCAGCAATCAGGA
CAAAAGCTCTCTATCCCCCAAATTACTACAAAGCATAGAGGGCTCTATACTTGCTCTGTTCGTAACTCAG
CTACTGGCAAGGAAAGCTCCAAATCCATGACAGTCGAAGTCTCTGCTCCTTCAGGAATAGGACGTCTTCC
TCTCCTTAATCCAATATAGCAGCCGTGAAGTCATTTCTGTATTTCAGGAAGACTGGCAGACAGTTGCTTT
GATTCTTCCTCAAACTACTTACAATCACCTACAGTCCAAAATTGCTTTTTCTTCAAGGAGATTTATGGAA
AAGACTCTGACAAGGACTCTTGAATACAAGTTCCTGATAACTTCAAGATCATACCACTGGACTAAGAACT
TTCAAAATTTTAATGAACAGGCTGATACCTTCATGAAATTCTAGACAAAGAAGAAAAAAACTCCATGTTA
TTGGACTAAATAATCAAAAGCATAATGTTTTCATAATTTTCTATTTGAAAATGTGCTGATTCTTTGAATG
TTTTATTCTCCAGATTTATGAACTTTTTTTCTTGAGCAATTGGTAAAGTATACTTTTGTAAACAAAAATT
GAAACATTTGCTTTTGCTCTCTATCTGAGTGCCCCAGAATTGGGAAACTATTCATGAGTATTCATATGTT
TATGGTAATAAAGTTATCTGCACAAGTTCA
Homo sapiens pregnancy specific beta-1-glycoprotein 6 (PSG6), transcript variant 1, mRNA
(NCBI Reference Sequence: NM_002782.5)
(SEQ ID NO: 6)
AGCACAGAAGGAGGAAGGACAGCACACCTGACAGCCCTGCTCAGGAAGTCTCTGGATCCTAGGCTCATCT
CCACAGGGGAGAACACACAGACAGCAGAGACCATGGGACCCCTCTCAGCCCCTCCCTGCACTCAGCACAT
CACCTGGAAGGGGCTCCTGCTCACAGCATCACTTTTAAACTTCTGGAACCTGCCCACCACTGCCCAAGTA
ATAATTGAAGCCAAGCCACCCAAAGTTTCCGAGGGGAAGGATGTTCTTCTACTTGTCCACAATTTGCCCC
AGAATCTTACTGGCTACATCTGGTACAAAGGGCAAATGACGGACCTCTACCATTACATTACATCATATGT
AGTACACGGTCAAATTATATATGGGCCTGCCTACAGTGGACGAGAAACAGTATATTCCAATGCATCCCTG
CTGATCCAGAATGTCACACAGGAGGATGCAGGATCCTACACCTTACACATCATAAAGCGAGGCGATGGGA
CTGGAGGAGTAACTGGATATTTCACTGTCACCTTATACTCGGAGACTCCCAAGCCCTCCATCTCCAGCAG
CAACTTAAACCCCAGGGAGGTCATGGAGGCTGTGCGCTTAATCTGTGATCCTGAGACTCCGGATGCAAGC
TACCTGTGGTTGCTGAATGGTCAGAACCTCCCTATGACTCACAGGTTGCAGCTGTCCAAAACCAACAGGA
CCCTCTATCTATTTGGTGTCACAAAGTATATTGCAGGACCCTATGAATGTGAAATACGGAACCCAGTGAG
TGCCAGCCGCAGTGACCCAGTCACCCTGAATCTCCTCCCGAAGCTGCCCATGCCTTACATCACCATCAAC
AACTTAAACCCCAGGGAGAAGAAGGATGTGTTAGCCTTCACCTGTGAACCTAAGAGTCGGAACTACACCT
ACATTTGGTGGCTAAATGGTCAGAGCCTCCCGGTCAGTCCGAGGGTAAAGCGACCCATTGAAAACAGGAT
ACTCATTCTACCCAGTGTCACGAGAAATGAAACAGGACCCTATCAATGTGAAATACGGGACCGATATGGT
GGCATCCGCAGTAACCCAGTCACCCTGAATGTCCTCTATGGTCCAGACCTCCCCAGAATTTACCCTTCAT
TCACCTATTACCGTTCAGGAGAAAACCTCGACTTGTCCTGCTTTGCGGACTCTAACCCACCGGCAGAGTA
TTCTTGGACAATTAATGGGAAGTTTCAGCTATCAGGACAAAAGCTCTTTATCCCCCAAATTACTACAAAT
CATAGCGGGCTCTATGCTTGCTCTGTTCGTAACTCAGCCACTGGCAAGGAAATCTCCAAATCCATGATAG
TCAAAGTCTCTGAGACAGCATCTCCCCAGGTTACCTATGCTGGTCCAAACACCTGGTTTCAAGAAATCCT
TCTGCTGTGACCTCCCAAAGTGCTAGGATTAAAACATGACCCACCATG
Homo sapiens pregnancy specific beta-1-glycoprotein 7 (PSG7), transcript variant 1, coding,
mRNA (NCBI Reference Sequence: NM_002783.3)
(SEQ ID NO: 7)
AGAAGTGCTCCTGCCCCGGGAAGAGGCTCAGTGCAGAAGGAGGAAGGACAGCACAGCTGACAGCCGTGCT
CAGGAAGATTCTGGATCCTAGGCTCATCTCCACAGAGGAGAACACGCAGGGAGCAGAGACCATGGGGCCC
CTCTCAGCCCCTCCCTGCACACAGCATATAACCTGGAAAGGGCTCCTGCTCACAGCATCACTTTTAAACT
TCTGGAACCCGCCCACCACAGCCCAAGTCACGATTGAAGCCCAGCCACCAAAAGTTTCCGAGGGGAAGGA
TGTTCTTCTACTTGTCCACAATTTGCCCCAGAATCTTACTGGCTACATCTGGTACAAAGGACAAATCAGG
GACCTCTACCATTATGTTACATCATATATAGTAGACGGTCAAATAATTAAATATGGGCCTGCATACAGTG
GACGAGAAACAGTATATTCCAATGCATCCCTGCTGATCCAGAATGTCACCCAGGAAGACACAGGATCCTA
CACTTTACACATCATAAAGCGAGGTGATGGGACTGGAGGAGTAACTGGACGTTTCACCTTCACCTTATAC
CTGGAGACTCCCAAACCCTCCATCTCCAGCAGCAATTTCAACCCCAGGGAGGCCACGGAGGCTGTGATTT
TAACCTGTGATCCTGAGACTCCAGATGCAAGCTACCTGTGGTGGATGAATGGTCAGAGCCTCCCTATGAC
TCACAGCTTGCAGCTGTCTGAAACCAACAGGACCCTCTACCTATTTGGTGTCACAAACTATACTGCAGGA
CCCTATGAATGTGAAATACGGAACCCAGTGAGTGCCAGCCGCAGTGACCCAGTCACCCTGAATCTCCTCC
CGAAGCTGCCCAAGCCCTACATCACCATCAATAACTTAAACCCCAGGGAGAATAAGGATGTCTCAACCTT
CACCTGTGAACCTAAGAGTGAGAACTACACCTACATTTGGTGGCTAAATGGTCAGAGCCTCCCGGTCAGT
CCCAGGGTAAAGCGACGCATTGAAAACAGGATCCTCATTCTACCCAGTGTCACGAGAAATGAAACAGGAC
CCTATCAATGTGAAATACGGGACCGATATGGTGGCATCCGCAGTGACCCAGTCACCCTGAATGTCCTCTA
TGGTCCAGACCTCCCCAGAATTTACCCTTCATTCACCTATTACCATTCAGGACAAAACCTCTACTTGTCC
TGCTTTGCGGACTCTAACCCACCGGCACAGTATTCTTGGACAATTAATGGGAAGTTTCAGCTATCAGGAC
AAAAGCTTTCTATCCCCCAGATTACTACAAAGCATAGCGGGCTCTATGCTTGCTCTGTTCGTAACTCAGC
CACTGGCAAGGAAAGCTCCAAATCCGTGACAGTCAGAGTCTCTGACTGGACATTACCCTGAATTCTACTA
GTTCCTCCAATTCCATCTTCTCCCATGGAACCTCAAAGAGCAAGACCCACTCTGTTCCAGAAGCCCTATA
AGTCAGAGTTGGACAACTCAATGTAAATTTCATGGGAAAATCCTTGTACCTGATGTCTGAGCCACTCAGA
ACTCACCAAAATGTTCAACACCATAACAACAGCTGCTCAAACTGTAAACAAGGAAAACAAGTTGATGACT
TCACACTGTGGACAGCTTTTCCCAAGATGTCAGAATAAGACTCCCCATCATGATGAGGCTCTCACCCCTC
TTAGCTGTCCTTGCTTGTGCCTGCCTCTTTCACTTGGCAGGATAATGCAGTCATTAGAATTTCACATGTA
GTATAGGAGCTTCTGAGGGTAACAACAGAGTGTCAGATATGTCATCTCAACCTCAGACTTTTACATAACA
TCTCAGGAGGAAATGTGGCTCTCTCCATCTTGCATACAGGGCTCCCAATAGAAATGAACACAGAGATATT
GCCTGTGTGTTTGCAGAGAAGATGGTTTCTATAAAGAGTAGGAAAGCTGAAATTATAGTAGACTCCCCTT
TAAATGCACATTGTGTGGATGGCTCTCACCATTTCCTAAGAGATACATTGTAAAACGTGACAGTAAGACT
GATTCTAGCAGAATAAAACATGTACTACATTTGCTAA
Homo sapiens pregnancy specific beta-1-glycoprotein 8 (PSG8), transcript variant 1, mRNA
(NCBI Reference Sequence: NM_182707.3)
(SEQ ID NO: 8)
AGAAGGAGGCAGGACAGCACTGCTGAGAGCTGTGCTCAGGAAGCTTCTGGATCCTAGGCTCATCTCCACA
GAGGAGAACACACAGACAGCAGAGACCATGGGGCTCCTCTCAGCCCCTCCCTGCACACAGCGCATCACCT
GGAAGGGGCTCCTGCTCACAGCATCACTTTTAAACTTCTGGAACCCACCCACGACTGCCCAAGTCACGAT
TGAAGCCCAGCCAACCAAAGTTTCTGAGGGGAAGGATGTTCTTCTACTTGTCCACAATTTGCCCCAGAAT
CTTACTGGCTACATCTGGTACAAAGGGCAAATCAGGGACCTCTACCATTACATTACATCATATGTAGTAG
ACGGTCAAATAATTATATATGGGCCTGCATACAGTGGACGAGAAACAATATATTCCAATGCATCCCTGCT
GATCCAGAATGTCACCCAGGAAGACGCAGGATCCTACACCTTACACATCATAATGGGAGGTGATGAGAAT
AGAGGAGTAACTGGACATTTCACCTTCACCTTATATCTGGAGACTCCCAAGCCCTCCATCTCCAGCAGCA
AATTAAACCCCAGGGAGGCCATGGAGGCTGTGAGCTTAACCTGTGATCCTGAGACTCCGGACGCAAGCTA
CCTGTGGTGGATGAATGGTCAGAGCCTCCCTATGTCTCACAGGTTGCAGTTGTCTGAAACCAACAGGACC
CTCTTTCTATTGGGTGTCACAAAGTACACTGCAGGACCCTATGAATGTGAAATACGGAACCCAGTGAGTG
CCAGCCGCAGTGACCCATTCACCCTGAATCTCCTCCCGAAGCTGCCCAAGCCCTACATCACCATCAACAA
CTTAAAACCCAGGGAGAATAAGGATGTCTTAAACTTCACCTGTGAACCTAAGAGTGAGAACTACACCTAC
ATTTGGTGGCTAAATGGTCAGAGCCTCCCGGTCAGTCCCAGGGTAAAGCGACCCATTGAAAACAGGATCC
TCATTCTACCCAGTGTCACGAGAAATGAAACAGGACCCTATCAATGTGAAATAAGGGACCAATATGGTGG
CATCCGCAGTTACCCAGTCACCCTGAATGTCCTCTATGGTCCAGACCTCCCCAGAATTTACCCTTCATTC
ACCTATTACCGTTCAGGAGAAGTCCTCTACTTGTCCTGTTCTGCGGACTCTAACCCACCGGCACAGTATT
CTTGGACAATTAATGGGAAGTTTCAGCTATCAGGACAAAAGCTCTTTATCCCCCAAATTACTACAAAGCA
TAGCGGGCTCTATGCTTGCTCTGTTCGTAACTCAGCCACTGGCAAGGAAAGCTCCAAATCCATGACAGTA
AAAGTCTCTGGTAAGCGGATCCCAGTATCCTTGGCAATAGGGATTTAGGTGGAGTCTATCTGGCCTTCAG
GGAAGAGTCAGGAAAACATTTTTATTCCCAGCCTGCGTCC
Homo sapiens pregnancy specific beta-1-glycoprotein 9 (PSG9), transcript variant 1, mRNA
(NCBI Reference Sequence: NM_002784.5)
(SEQ ID NO: 9)
ACAGAAGGAGGAAGGACAGCACAGCTGACAGCCGTGCTCAGACAGCTTCTGGATCCCAGGCTCATCTCCA
CAGAGGAGAACACACAGGCAGCAGAGACCATGGGGCCCCTCCCAGCCCCTTCCTGCACACAGCGCATCAC
CTGGAAGGGGCTCCTGCTCACAGCATCACTTTTAAACTTCTGGAACCCGCCCACCACTGCCGAAGTCACG
ATTGAAGCCCAGCCACCCAAAGTTTCTGAGGGGAAGGATGTTCTTCTACTTGTCCACAATTTGCCCCAGA
ATCTTCCTGGCTACTTCTGGTACAAAGGGGAAATGACGGACCTCTACCATTACATTATATCGTATATAGT
TGATGGTAAAATAATTATATATGGGCCTGCATACAGTGGAAGAGAAACAGTATATTCCAACGCATCCCTG
CTGATCCAGAATGTCACCCGGAAGGATGCAGGAACCTACACCTTACACATCATAAAGCGAGGTGATGAGA
CTAGAGAAGAAATTCGACATTTCACCTTCACCTTATACTTGGAGACTCCCAAGCCCTACATCTCCAGCAG
CAACTTAAACCCCAGGGAGGCCATGGAGGCTGTGCGCTTAATCTGTGATCCTGAGACTCTGGACGCAAGC
TACCTATGGTGGATGAATGGTCAGAGCCTCCCTGTGACTCACAGGTTGCAGCTGTCCAAAACCAACAGGA
CCCTCTATCTATTTGGTGTCACAAAGTATATTGCAGGACCCTATGAATGTGAAATACGGAACCCAGTGAG
TGCCAGTCGCAGTGACCCAGTCACCCTGAATCTCCTCCCGAAGCTGCCCATCCCCTACATCACCATCAAC
AACTTAAACCCCAGGGAGAATAAGGATGTCTTAGCCTTCACCTGTGAACCTAAGAGTGAGAACTACACCT
ACATTTGGTGGCTAAACGGTCAGAGCCTCCCCGTCAGTCCCGGGGTAAAGCGACCCATTGAAAACAGGAT
ACTCATTCTACCCAGTGTCACGAGAAATGAAACAGGACCCTATCAATGTGAAATACGGGACCGATATGGT
GGCCTCCGCAGTAACCCAGTCATCCTAAATGTCCTCTATGGTCCAGACCTCCCCAGAATTTACCCTTCAT
TCACCTATTACCGTTCAGGAGAAAACCTCGACTTGTCCTGCTTCACGGAATCTAACCCACCGGCAGAGTA
TTTTTGGACAATTAATGGGAAGTTTCAGCAATCAGGACAAAAGCTCTTTATCCCCCAAATTACTAGAAAT
CATAGCGGGCTCTATGCTTGCTCTGTTCATAACTCAGCCACTGGCAAGGAAATCTCCAAATCCATGACAG
TCAAAGTCTCTGGTCCCTGCCATGGAGACCTGACAGAGTCTCAGTCATGACTGCAACAACTGAGACACTG
AGAAAAAGAACAGGCTGATACCTTCATGAAATTCAAGACAAAGAAGAAAAAAACTCAATGTTATTGGACT
AAATAATCAAAAGGATAATGTTTTCATAATTTTTTATTGGAAAATGTGCTGATTCTTTGAATGTTTTATT
CTCCAGATTTATGAACTTTTTTTCTTCAGCAATTGGTAAAGTATACTTTTGTAAACAAAAATTGAAATAT
TTGCTTTTGCTGTCTATCTGAATGCCCCAGAATTGTGAAACTATTCATGAGTATTCATAGGTTTATGGTA
ATAAAGTTATTTGCACATGTTCCGTAA
Homo sapiens pregnancy specific beta-1-glycoprotein 10 (PSG10), mRNA (NCBI Reference
Sequence: NM_176809.2)
(SEQ ID NO: 10)
GGGTGGATCCTAGGCTCATCTCCATAGGGGAGAACACACATACAGCAGAGACCATGGGACCCCTCTCAGC
CCCTCCCTGCACTCAGCACATCACCTGGAAGGGGCTCCTGCTCACAGCATCACTTTTAAACTTCTGGAAC
CTGCCCACCACTGCCCAAGTAATAATTGAAGCCCAGCCACCCAAAGTTTCTGAGGGGAAGGATGTTCTTC
TACTTGTCCACAATTTGCCCCAGAATCTTACTGGCTACATCTGGTACAAAGGGCAAATGACGGACCTCTA
CCATTACATTACATCATATGTAGTAGACGGTCAAATTATATATGGGCCTGCCTACAGTGGACGAGAAACA
GTATATTCCAATGCATCCCTGCTGATCCAGAATGTCACACAGGAGGATGCAGGATCCTACACCTTACACA
TCATAAAGCGAGGCGATGGGACTGGAGGAGTAACTGGATATTTCACTGTCACCTTATACTCGGAGACTCC
CAAGCGCTCCATCTCCAGCAGCAACTTAAACCCCAGGGAGGTCATGGAGGCTGTGCGCTTAATCTGTGAT
CCTGAGACTCCGGATGCAAGCTACCTGTGGTTGCTGAATGGTCAGAACCTCCCTATGACTCACAGGTTGC
AGCTGTCCAAAACCAACAGGACCCTCTATCTATTTGGTGTCACAAAGTATATTGCAGGGCCCTATGAATG
TGAAATACGGAGGGGAGTGAGTGCCAGCCGCAGTGACCCAGTCACCCTGAATCTCCTCCCGAAGCTGCCC
ATGCCTTACATCACCATCAACAACTTAAACCCCAGGGAGAAGAAGGATGTGTTAGCCTTCACCTGTGAAC
CTAAGAGTCGGAACTACACCTACATTTGGTGGCTAAATGGTCAGAGCCTCCCGGTCAGTCCGAGGGTAAA
GCGACCCATTGAAAACAGGATACTCATTCTACCCAGTGTCACGAGAAATGAAACAGGACCCTATCAATGT
GAAATACGGGACCGATATGGTGGCATCCGCAGTAACCCAGTCACCCTGAATGTCCTCTATGGTCCAGACC
TCCCCAGAATTTACCCTTACTTCACCTATTACCGTTCAGGAGAAAACCTCGACTTGTCCTGCTTTGCGGA
CTCTAACCCACCGGCAGAGTATTTTTGGACAATTAATGGGAAGTTTCAGCTATCAGGACAAAAGCTCTTT
ATCCCCCAAATTACTACAAATCATAGCGGGCTCTATGCTTGCTCTGTTCGTAACTCAGCCACTGGCAAGG
AAATCTCCAAATCCATGATAGTCAAAGTCTCTGGTCCCTGCCATGGAAACCAGACAGAGTCTCATTAATG
GCTGCCACAATAGAGACACTGAGAAAAAGAACAGGTTGATACCTTCATGAAATTCAAGACAAAGAAGAAA
AAGGCTCAATGTTATTGGACTAAATAATCAAAAGGATAATGTTTTCATAATTTTTATTGGAAAATGTGCT
GATTCTTGGAATGTTTTATTCTCCAGATTTATGAACTTTTTTTCTTCAGCAATTGGTAAAGTATACTTTT
GTAAACAAAAATTGAAACATTTGCTTTTGCTCTCTATCTGAGTGCCCCCCC
Homo sapiens pregnancy specific beta-1-glycoprotein 11 (PSG11), transcript variant 1,
mRNA (NCBI Reference Sequence: NM_002785.3)
(SEQ ID NO: 11)
AGAAGGAGGAAGGACAGCACAGCTGACAGCCGTGCTCTGGAAGCTTCTGGATCCTAGGCTCATCTCCACA
GAGGAGAACATGCACGCAGCAGAGATCATGGGGCCCCTCTCAGCCCCTCCCTGCACAGAGCACATCAAAT
GGAAGGGGCTCCTGCTCACAGCATTACTTTTAAACTTCTGGAACTTGCCTACCACTGCCCAAGTCATGAT
TGAAGCCCAGCCACCCAAAGTGTCCGAGGGGAAGGATGTTCTTCTACTTGTCCACAATTTGCCCCAGAAT
CTTACTGGCTACATCTGGTACAAAGGGCAAATCAGGGACCTCTACCATTACATTACATCATATGTAGTAG
ACGGTCAAATAATTATATATGGACCGGCATACAGTGGACGAGAAACAGTATATTCCAATGCATCCCTGCT
GATCCAGAATGTCACCCGGGAGGACGCAGGATCCTACACCTTACACATCATAAAGCGAGGTGATGGGACT
AGAGGAGTAACTGGATATTTCACCTTCACCTTATACCTGGAGACTCCCAAGCCCTCCATCTCCAGCAGCA
ACTTAAACCCCAGGGAGGCCATGGAGACTGTGATCTTAACCTGTAATCCTGAGACTCCGGACGCAAGCTA
CCTGTGGTGGATGAATGGTCAGAGCCTCCCTATGACTCATAGGATGCAGCTGTCTGAAACCAACAGGACC
CTCTTTCTATTTGGTGTCACAAAGTATACTGCAGGACCCTATGAATGTGAAATATGGAACTCAGGGAGTG
CCAGCCGCAGTGACCCAGTCACCCTGAATCTCCTCCATGGTCCAGACCTCCCCAGAATTTTCCCTTCAGT
CACCTCTTACTATTCAGGAGAGAACCTCGACTTGTCCTGCTTCGCAAACTCTAACCCACCAGCACAGTAT
TCTTGGACAATTAATGGGAAGTTTCAGCTATCAGGACAAAAGCTCTTTATCCCTCAGATTACTCCAAAGC
ATAATGGGCTCTATGCTTGCTCTGCTCGTAACTCAGCCACTGGCGAGGAAAGCTCCACATCCTTGACAAT
CAGAGTCATTGCTCCTCCAGGATTAGGAACTTTTGCTTTCAATAATCCAACGTAGCAGCCGTGATGTCAT
TTTTGTATTTCAGGAAGACTGGCAGGAGATTTATGGAAAAGACTATGAAAAGGACTCTTGAATACAAGTT
CCTGATAACTTCAAGATCATACCACTGGACTAAGAACTTTCAAAATTTTGATGAACAGGCTGATACCTTC
ATGAAATTCAAGACAAAGAAGAAAAGAACTCCATTTCATTGGACTAAATAACAAAAGGATAATGTTTTCA
TAATTTTTTATTGGAAAATGTGCTGATTTTTTGAATGTTTTATCCTCCAGATTTATGAATTTTTTTCTTC
AGCAATTGGTAAAGTATACTTTTGTAAACAAAAATTGAAACATTTGCTTTTGCTCTCTGAGTGCCCCAGA
ATTGGGAATCTATTCATGAATATTCATATGTTTATGGTAATAAAGTTATTTGCACAAGTTTA

In one aspect, the present disclosure provides PSG-specific inhibitory nucleic acids comprising a nucleic acid molecule which is complementary to a portion of a PSG nucleic acid sequence (e.g., a PSG nucleic acid sequence selected from the group consisting of SEQ ID NOs: 1-11).

The present disclosure also provides an antisense nucleic acid comprising a nucleic acid sequence that is complementary to and specifically hybridizes with a portion of any one of a PSG mRNA (e.g., a PSG mRNA sequence selected from the group consisting of SEQ ID NOs: 1-11), thereby reducing or inhibiting expression of one or more PSGs. The antisense nucleic acid may be antisense RNA, or antisense DNA. Antisense nucleic acids based on the known PSG gene sequence can be readily designed and engineered using methods known in the art.

Antisense nucleic acids are molecules which are complementary to a sense nucleic acid strand, e.g., complementary to the coding strand of a double-stranded DNA molecule (or cDNA) or complementary to an mRNA sequence. Accordingly, an antisense nucleic acid can form hydrogen bonds with a sense nucleic acid. The antisense nucleic acid can be complementary to an entire PSG coding strand, or to a portion thereof, e.g., all or part of the protein coding region (or open reading frame). In some embodiments, the antisense nucleic acid is an oligonucleotide which is complementary to only a portion of the coding region of a PSG mRNA. In certain embodiments, an antisense nucleic acid molecule can be complementary to a noncoding region of the PSG coding strand In some embodiments, the noncoding region refers to the 5′ and 3′ untranslated regions that flank the coding region and are not translated into amino acids. For example, the antisense oligonucleotide can be complementary to the region surrounding the translation start site of a PSG gene. An antisense oligonucleotide can be, for example, about 5, 10, 15, 20, 25, 30, 35, 40, 45 or 50 nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or modified nucleotides designed to increase the biological stability of the molecules or to increase the physical stability of the duplex formed between the antisense and sense nucleic acids, e.g., phosphorothioate derivatives and acridine substituted nucleotides. Examples of modified nucleotides which can be used to generate the antisense nucleic acid include 5-fluorouracil, 5-bromouracil, 5-chlorouracil, 5-hodouracil, hypoxanthine, xanthine, 4-acetylcytosine, 5-(carboxyhydroxylmethyl)uracil, 5-carboxymethylaminomethyl-2-thouridine, 5-carboxymethylaminometh-yluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutosine, pseudouracil, queosine, 2-thiocytosine, 5-methyl-2-thiouracil, 2-thlouracil, 4-thiouracil, 5-methyluracil, uracil-5-oxyacetic acid methylester, uracil-5-cxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest)

The antisense nucleic acid molecules may be administered to a subject or generated in situ such that they hybridize with or bind to cellular mRNA and/or genomic DNA encoding the protein of interest to thereby inhibit expression of the protein, e.g., by inhibiting transcription and/or translation. The hybridization can occur via Watson-Crick base pairing to form a stable duplex, or in the case of an antisense nucleic acid molecule which binds to DNA duplexes, through specific interactions in the major groove of the double helix.

In some embodiments, the antisense nucleic acid molecules are modified such that they specifically bind to receptors or antigens expressed on a selected cell surface, e.g., by linking the antisense nucleic acid molecules to peptides or antibodies which bind to cell surface receptors or antigens. In some embodiments, the antisense nucleic acid molecule is an alpha-anomeric nucleic acid molecule. An alpha-anomeric nucleic acid molecule forms specific double-stranded hybrids with complementary RNA in which, contrary to the usual s-units, the strands run parallel to each other (Gaultier et al., Nucleic Acids. Res. 15:6625-6641(1987)). The antisense nucleic acid molecule can also comprise a 2′-O-methylribonucleotide (Inoue et al., Nucleic Acids Res 15.6131-6148 (1987)) or a chimeric RNA-DNA analogue (Inoue et al., FFBS Lett. 215:327-330 (1987)).

The present disclosure also provides a short hairpin RNA (shRNA) or small interfering RNA (siRNA) comprising a nucleic acid sequence that is complementary to and specifically hybridizes with a portion of a PSG mRNA (e.g., a PSG mRNA selected from among any one of SEQ ID NOs: 1-11), thereby reducing or inhibiting expression of one or more PSG genes. In some embodiments, the shRNA or siRNA is about 18, 19, 20, 21, 22, 23, 24, 25, 26, 27, 28 or 29 base pairs in length. Double-stranded RNA (dsRNA) can induce sequence-specific post-transcriptional gene silencing (e.g., RNA interference (RNAi)) in many organisms such as C. elegans, Drosophila, plants, mammals, oocytes and early embryos. RNAi is a process that interferes with or significantly reduces the number of protein copies made by an mRNA. For example, a double-stranded siRNA or shRNA molecule is engineered to complement and hydridize to a mRNA of a target gene Following intracellular delivery, the siRNA or shRNA molecule associates with an RNA-induced silencing complex (RISC), which then binds and degrades a complementary target mRNA (such as a PSG mRNA)

The present disclosure also provides a ribozyme comprising a nucleic acid sequence that is complementary to and specifically hybridizes with a portion of a PSG mRNA (e.g., a PSG mRNA selected from among any one of SEQ ID NOs: 1-11), thereby reducing or inhibiting expression of one or more PSG genes. Ribozymes are catalytic RNA molecules with ribonuclease activity which are capable of cleaving a complementary single-stranded nucleic acid, such as an mRNA. Thus, ribozymes (e.g., hammerhead ribozymes (described in Haselhoff and Gerlach, Nature 334:585-591 (1988))) can be used to catalytically cleave PSG transcripts, thereby inhibiting translation of one or more PSG genes.

A ribozyme having specificity for a PSG-encoding nucleic acid can be designed based upon a PSG nucleic acid sequence disclosed herein. For example, a derivative of a Tetrahymena L-19 IVS RNA can be constructed in which the nucleotide sequence of the active site is complementary to the nucleotide sequence to be cleaved in a PSG-encoding mRNA. See, e.g., U.S. Pat. Nos. 4,987,071 and 5,116,742. Alternatively, a PSG mRNA can be used to select a catalytic RNA having a specific ribonuclease activity from a pool of RNA molecules. See, e.g., Bartel and Szostak (1993) Science 261:1411-1418, incorporated herein by reference.

The present disclosure also provides a synthetic guide RNA (sgRNA) comprising a nucleic acid sequence that is complementary to and specifically hybridizes with a portion of a PSG nucleic acid (e.g., a PSG nucleic acid selected from among any one of SEQ ID NOs: 1-11). Guide RNAs for use in CRISPR-Cas systems are typically generated as a single guide RNA comprising a crRNA segment and a tracrRNA segment. The crRNA segment and a tracrRNA segment can also be generated as separate RNA molecules. The crRNA segment comprises the targeting sequence that binds to a portion of a PSG nucleic acid (e.g., a PSG nucleic acid selected from among any one of SEQ ID NOs: 1-11), and a stem portion that hybridizes to a tracrRNA. The tracrRNA segment comprises a nucleotide sequence that is partially or completely complementary to the stem sequence of the crRNA and a nucleotide sequence that binds to the CRISPR enzyme. In some embodiments, the crRNA segment and the tracrRNA segment are provided as a single guide RNA. In some embodiments, the crRNA segment and the tracrRNA segment are provided as separate RNAs. The combination of the CRISPR enzyme with the crRNA and tracrRNA make up a functional CRISPR-Cas system. Exemplary CRISPR-Cas systems for targeting nucleic acids, are described, for example, in WO2015/089465

In some embodiments, a synthetic guide RNA is a single RNA represented as comprising the following elements: 5′-X1-X2-Y-Z-3′ where X1 and X2 represent the crRNA segment, where X1 is the targeting sequence that binds to a portion of a PSG nucleic acid (e.g., a PSG nucleic acid selected from among any one of SEQ ID NOs: 1-11), X2 is a stem sequence the hybridizes to a tracrRNA, Z represents a tracrRNA segment comprising a nucleotide sequence that is partially or completely complementary to X2, and Y represents a linker sequence. In some embodiments, the linker sequence comprises two or more nucleotides and links the crRNA and tracrRNA segments. In some embodiments, the linker sequence comprises 2, 3, 4, 5, 6, 7, 8, 9, 10 or more nucleotides. In some embodiments, the linker is the loop of the hairpin structure formed when the stem sequence hybridized with the tracrRNA.

In some embodiments, a synthetic guide RNA is provided as two separate RNAs where one RNA represents a crRNA segment: 5′-X1-X2-3′ where X1 is the targeting sequence that binds to a portion of a PSG nucleic acid (e.g., a PSG nucleic acid selected from among any one of SEQ ID NOs: 1-11), X2 is a stem sequence the hybridizes to a tracrRNA, and one RNA represents a tracrRNA segment, Z, that is a separate RNA from the crRNA segment and comprises a nucleotide sequence that is partially or completely complementary to X2 of the crRNA.

Exemplary crRNA stem sequences and tracrRNA sequences are provided, for example, in WO/2015/089465, which is incorporated by reference herein. In general, a stem sequence includes any sequence that has sufficient complementarity with a complementary sequence in the tracrRNA to promote formation of a CRISPR complex at a target sequence, wherein the CRISPR complex comprises the stem sequence hybridized to the tracrRNA. In general, degree of complementarity is with reference to the optimal alignment of the stem and complementary sequence in the tracrRNA, along the length of the shorter of the two sequences. Optimal alignment may be determined by any suitable alignment algorithm, and may further account for secondary structures, such as self-complementarity within either the stem sequence or the complementary sequence in the tracrRNA. In some embodiments, the degree of complementarity between the stem sequence and the complementary sequence in the tracrRNA along the length of the shorter of the two when optimally aligned is about or more than about 25%, 30%, 40%, 50%, 60%, 70%, 80%, 90%, 95%, 97.5%, 99%, or higher. In some embodiments, the stem sequence is about or more than about 5, 6, 7, 8, 9, 10, 11, 12, 13, 14, 15, 16, 17, 18, 19, 20, 25, 30, 40, 50, or more nucleotides in length. In some embodiments, the stem sequence and complementary sequence in the tracrRNA are contained within a single RNA, such that hybridization between the two produces a transcript having a secondary structure, such as a hairpin. In some embodiments, the tracrRNA has additional complementary sequences that form hairpins. In some embodiments, the tracrRNA has at least two or more hairpins. In some embodiments, the tracrRNA has two, three, four or five hairpins. In some embodiments, the tracrRNA has at most five hairpins.

In a hairpin structure, the portion of the sequence 5′ of the final “N” and upstream of the loop corresponds to the crRNA stem sequence, and the portion of the sequence 3′ of the loop corresponds to the tracrRNA sequence. Further non-limiting examples of single polynucleotides comprising a guide sequence, a stem sequence, and a tracr sequence are as follows (listed 5′ to 3′), where “N” represents a base of a guide sequence (e.g. a modified oligonucleotide provided herein), the first block of lower case letters represent stem sequence, and the second block of lower case letters represent the tracrRNA sequence, and the final poly-T sequence represents the transcription terminator: (a) NNNNNNNNNNNNNNNNNNNNgtttttgtactctcaagatttaGAAAtaaatcttgcagaagctacaaagataa ggcttcatgccgaaatcaacaccctgtcattttatggcagggtgttttcgttatttaaTTTTTT (SEQ ID NO: 12); (b) NNNNNNNNNNNNNNNNNNNNgtttttgtactctcaGAAAtgcagaagctacaaagataaggcttcatgccg aaatcaacaccctgtcattttatggcagggtgttttcgttatttaaTTTTTT (SEQ ID NO: 13); (c) NNNNNNNNNNNNNNNNNNNNgtttttgtactctcaGAAAtgcagaagctacaaagataaggcttcatgccg aaatcaacaccctgtcattttatggcagggtgtTTTTTT (SEQ ID NO: 14); (d) NNNNNNNNNNNNNNNNNNNNgttttagagctaGAAAtagcaagttaaaataaggctagtccgttatcaactt gaaaaagtggcaccgagtcggtgcTTTTTT (SEQ ID NO: 15); (e) NNNNNNNNNNNNNNNNNNNNgttttagagctaGAAATAGcaagttaaaataaggetagtecgttatcaac ttgaaaaagtgTTTTT (SEQ ID NO: 16); and (f) NNNNNNNNNNNNNNNNNNNNgttttagagctagAAATAGcaagttaaaataaggctagtccgttatcaTT TTTTTT (SEQ ID NO: 17).

Selection of suitable oligonucleotides for use in as a targeting sequence in a CRISPR Cas system depends on several factors including the particular CRISPR enzyme to be used and the presence of corresponding proto-spacer adjacent motifs (PAMs) downstream of the target sequence in the target nucleic acid. The PAM sequences direct the cleavage of the target nucleic acid by the CRISPR enzyme. In some embodiments, a suitable PAM is 5′-NRG or 5′-NNGRR (where N is any Nucleotide) for SpCas9 or SaCas9 enzymes (or derived enzymes), respectively. Generally the PAM sequences should be present between about 1 to about 10 nucleotides of the target sequence to generate efficient cleavage of the target nucleic acid. Thus, when the guide RNA forms a complex with the CRISPR enzyme, the complex locates the target and PAM sequence, unwinds the DNA duplex, and the guide RNA anneals to the complementary sequence on the opposite strand. This enables the Cas9 nuclease to create a double-strand break.

A variety of CRISPR enzymes are available for use in conjunction with the disclosed guide RNAs of the present disclosure. In some embodiments, the CRISPR enzyme is a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5. Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof. In some embodiments, the CRISPR enzyme cleaves both strands of the target nucleic acid at the Protospacer Adjacent Motif (PAM) site In some embodiments, the CRISPR enzyme is a nickase, which cleaves only one strand of the target nucleic acid.

Aptamers are macromolecules composed of nucleic acid (e.g., RNA, DNA) that bind tightly to a specific molecular target (e.g., PSG polypeptide or an epitope thereof). A particular aptamer may be described by a linear nucleotide sequence and is typically about 15-60 nucleotides in length. In some embodiments, aptamers are modified to dramatically reduce their sensitivity to degradation by enzymes in the blood for use in in vivo applications. In addition, aptamers can be modified to alter their biodistribution or plasma residence time.

Selection of aptamers that can bind a PSG gene or a fragment thereof can be achieved through methods known in the art. For example, aptamers can be selected using the SELEX (Systematic Evolution of Ligands by Exponential Enrichment) method (Tuerk, C, and Gold, L., Science 249:505-510 (1990): Jayasena, S. D. Clin. Chem. 45:1628-1650 (1999)).

### Anti-PSG Antibodies

The present disclosure encompasses the use of PSG-targeting antibodies in the treatment of cancers. Such antibodies include, but are not limited to, polyclonal antibodies: monoclonal antibodies or antigen binding fragments thereof, modified antibodies such as chimeric antibodies, reshaped antibodies, humanized antibodies, or fragments thereof (e.g., Fv, Fab′, Fab, F(ab′)₂); or biosynthetic antibodies, e.g., single chain antibodies, single domain antibodies (DAB), Fvs, or single chain Fvs (scFv). Methods of making and using polyclonal and monoclonal antibodies are well known in the art, e.g., in Harlow et al., Using Antibodies: A Laboratory Manual. Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)₂ fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000, 1st edition)

Examples of anti-PSG antibody agents useful in the methods disclosed herein include, but are not limited to monoclonal antibodies, human antibodies, humanized antibodies, multi-specific antibodies, bispecific antibodies, camelised antibodies, chimeric antibodies, antibody fragments (e.g., Fab, F(ab′)2, Fab′, scFv, Fv, Fd, dAB), single domain antibodies (e.g., nanobody, single domain camelid antibody), scFv-Fc. VNAR fragments, bispecific T-cell engager (BITE) antibodies, minibodies, antibody drug conjugates, fusion polypeptides, disulfide-linked Fvs (sdFvs), intrabodies, and anti-idiotypic antibodies.

Any anti-PSG antibody agents known in the art are useful in the methods disclosed herein. Examples of PSG antibodies include, but are not limited to MAB6799 (R&D Systems, Minneapolis, MN), BAP-3, BAP-1, PSG.01-PSG.16. mAbs (Houston, Aileen et al., mAbs vol. 8, 3 (2016): 491-500), etc.

### Small Molecule Inhibitors of PSG

Small molecule inhibitors of PSG gene expression are also within the scope of the present technology and may be identified by screening libraries of compounds using, for example, cell lines that express a PSG polypeptide or a version of a PSG polypeptide that has been modified to include a readily detectable moiety. Methods for identifying compounds capable of modulating gene expression are described, for example, in U.S. Pat. No. 5,976,793

In any and all embodiments of the methods disclosed herein, the subject is diagnosed as having, suspected as having, or at risk of having a disease or condition characterized by elevated expression levels and/or increased activity of PSG. Additionally or alternatively, in some embodiments, the subject is diagnosed as having cancer.

In any and all embodiments of the methods disclosed herein, the cancer is breast cancer, lung cancer, uterine cancer, colon cancer, rectal cancer, endometrial cancer, stomach cancer, intestinal cancer, renal cancer, acute lymphoblastic leukemia (ALL), chronic lymphocytic leukemia (CLL), acute myeloid leukemia (AML), and metastases thereof.

In therapeutic applications, compositions or medicaments comprising a PSG inhibitor disclosed herein are administered to a subject suspected of, or already suffering from such a disease or condition (such as, a subject diagnosed with a disease or condition characterized by elevated expression levels and/or increased activity of PSG and/or a subject diagnosed with cancer), in an amount sufficient to cure, or at least partially arrest, the symptoms of the disease, including its complications and intermediate pathological phenotypes in development of the disease.

Subjects suffering from a disease or condition characterized by elevated expression levels and/or increased activity of PSG and/or a subject diagnosed with cancer can be identified by any or a combination of diagnostic or prognostic assays known in the art.

In certain embodiments, subjects with a disease or condition characterized by elevated expression levels and/or increased activity of PSG, and/or subjects suffering from cancer that are treated with one or more PSG inhibitors will show reduced tumor proliferation and/or increased survival compared to untreated cancer patients. In certain embodiments, subjects with a disease or condition characterized by elevated expression levels and/or increased activity of PSG, and/or subjects suffering from cancer that are treated with the PSG inhibitors will show reduced PSG and/or CTLA-4 expression levels compared to untreated cancer patients.

In any and all embodiments of the methods disclosed herein, administration of the at least one PSG inhibitor results in one or more improvements in the subject selected among (a) reduced levels and/or activity of CTLA-4 expressing regulatory T cells, (b) inhibition of tumor cell proliferation and/or tumor metastasis, (c) reduced tumor size, (d) amelioration of cancer symptoms, (e) increased weight gain, (f) extended lifespan, (g) prolonged progression-free survival, and (h) decreased risk of cancer therapy-associated side-effects (e.g., autoimmunity).

For therapeutic applications, a composition comprising a PSG inhibitor disclosed herein, is administered to the subject. In some embodiments, the PSG inhibitor is administered one, two, three, four, or five times per day. In some embodiments, the PSG inhibitor is administered more than five times per day. Additionally or alternatively, in some embodiments, the PSG inhibitor is administered every day, every other day, every third day, every fourth day, every fifth day, or every sixth day. In some embodiments, the PSG inhibitor is administered weekly, bi-weekly, tri-weekly, or monthly. In some embodiments, the PSG inhibitor is administered for a period of one, two, three, four, or five weeks. In some embodiments, the PSG inhibitor is administered for six weeks or more. In some embodiments, the PSG inhibitor is administered for twelve weeks or more. In some embodiments, the PSG inhibitor is administered for a period of less than one year. In some embodiments, the PSG inhibitor is administered for a period of more than one year. In some embodiments, the PSG inhibitor is administered throughout the subject's life.

In some embodiments of the methods of the present technology, the PSG inhibitor is administered daily for 1 week or more. In some embodiments of the methods of the present technology, the PSG inhibitor is administered daily for 2 weeks or more. In some embodiments of the methods of the present technology, the PSG inhibitor is administered daily for 3 weeks or more. In some embodiments of the methods of the present technology, the PSG inhibitor is administered daily for 4 weeks or more. In some embodiments of the methods of the present technology, the PSG inhibitor is administered daily for 6 weeks or more. In some embodiments of the methods of the present technology, the PSG inhibitor is administered daily for 12 weeks or more. In some embodiments, the PSG inhibitor is administered daily throughout the subject's life.

### Determination of the Biological Effect of PSG Inhibitors of the Present Technology

In various embodiments, suitable in vitro or in vivo assays are performed to determine the effect of a specific PSG inhibitor and whether its administration is indicated for treatment. In various embodiments, in vitro assays can be performed with representative animal models, to determine if a given PSG inhibitor exerts the desired effect on reducing or eliminating signs and/or symptoms of cancer. Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects. In some embodiments, in vitro or in vivo testing is directed to the biological function of one or more PSG inhibitors.

Animal models of cancer may be generated using techniques known in the art. Such models may be used to demonstrate the biological effect of PSG inhibitors in the treatment of cancer, and for determining what comprises a therapeutically effective amount of the one or more PSG inhibitors disclosed herein in a given context.

### Modes of Administration and Effective Dosages

Any method known to those in the art for contacting a cell, organ or tissue with one or more PSG inhibitors disclosed herein may be employed. Suitable methods include in vitro, ex vivo, or in vivo methods. In vivo methods typically include the administration of one or more PSG inhibitors to a mammal, suitably a human. When used in vivo for therapy, the one or more PSG inhibitors described herein are administered to the subject in effective amounts (i.e., amounts that have desired therapeutic effect). The dose and dosage regimen will depend upon the degree of the disease state of the subject, the characteristics of the particular PSG inhibitor used, e.g., its therapeutic index, and the subject's history.

The effective amount may be determined during pre-clinical trials and clinical trials by methods familiar to physicians and clinicians. An effective amount of one or more PSG inhibitors useful in the methods may be administered to a mammal in need thereof by any of a number of well-known methods for administering pharmaceutical compounds. The PSG inhibitors may be administered systemically or locally.

The one or more PSG inhibitors described herein can be incorporated into pharmaceutical compositions for administration, singly or in combination, to a subject for the treatment of cancer. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more PSG inhibitors disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more PSG inhibitors disclosed herein sufficient for achieving a therapeutic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more PSG inhibitor concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more PSG inhibitors may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Combination Therapy

In some embodiments, one or more PSG inhibitors disclosed herein may be combined with one or more additional therapies for the treatment of cancer.

In some embodiments, the one or more PSG inhibitors disclosed herein may be separately, sequentially or simultaneously administered with at least one additional therapeutic agent. In some embodiments, the at least one additional therapeutic agent is selected from the group consisting of alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitor include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

In some embodiments, the one or more PSG inhibitors disclosed herein may be separately, sequentially or simultaneously administered with at least one additional immuno-modulating/stimulating antibody including but not limited to anti-PD-1 antibody, anti-PD-L1 antibody, anti-PD-L2 antibody, anti-CTLA-4 antibody, anti-TIM3 antibody, anti-4-1BB antibody, anti-CD73 antibody, anti-GITR antibody, and anti-LAG-3 antibody.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure also provides kits for the treatment of cancer comprising one or more PSG inhibitors disclosed herein. Optionally, the above described components of the kits of the present technology are packed in suitable containers and labeled for the treatment of cancer. In another aspect, the present disclosure provides a kit for enhancing anti-tumor immune responses in a cancer patient by testing for the presence of elevated levels or activity of one or more PSG genes, e.g., in a tumor sample, in the cancer patient. The kit can comprise, for example, an antibody for detection of a PSG polypeptide or a probe for detection of a PSG polynucleotide. In addition, the kit can include a reference or control sample, instructions for processing samples, performing the test and interpreting the results, buffers and other reagents necessary for performing the test. In one embodiment the kit comprises one or more PSG inhibitors described herein. In certain embodiments, the kit includes instructions for administering to the cancer patient one or more PSG inhibitors if elevated levels or activity of one or more PSG genes is detected in the tumor sample. Additionally or alternatively, in some embodiments, the kit includes instructions for administering to the cancer patient immune checkpoint blockade therapy (e.g., antibodies targeting CTLA-4, PDL1, PD1 etc.) if elevated levels or activity of one or more PSG genes is detected in the tumor sample.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

### EXAMPLES

### Example 1: General Experimental Methods

Overview. A central problem in the field of network science is the representation of network data in a readily accessible format (R. Albert, A. Barabasi, Rev. Mod. Phys. 74, 47-97 (2002)). Ideally the representation should be amenable to human-in-the-loop, interactive, exploratory data analysis. Compression methods have been used previously to reduce large networks to a desired level of resolution, mainly toward the goal of improving the computational performance of community detection algorithms (L. Yang et al., Sci. Rep. 7, 634 (2017)). The key idea is to group nodes into modules and consider the new network comprised of the connections between groups implied by the individual node connections. In its simplest form, the groups may be obtained by devising an edge weighting intended to measure similarity between neighboring nodes, and successively “collapsing” edges, beginning with the greatest similarity, to create a hierarchical representation.

A natural improvement is to find approximations of a given network “from below” with gradually expanding small node subsets, an approach developed by Stanley et al. Sci. Rep. 8, 10892 (2018) where the authors randomly distribute seed nodes which are then expanded into “supernodes” using direct neighborhoods. In a more global approach, Yang et al. Sci. Rep. 7, 634 (2017) present a method of supernode network representation involving explicit consideration of known prior constraints on the set of network topologies of interest for a low-complexity approximation to the given network satisfying the constraints. For a more detailed survey, see Besta et al. arXiv: 1806.01799 (5 Jun. 2018).

Unlike the method referred to in Stanley et al. (2018), embodiments of the disclosed approach make essential use of additional data beyond the network topology. Moreover, rather than qualitative constraints as in Yang et al. (2017), it is assumed that the nodes of the network are numerical feature variables, so the network is augmented with node weight data. For example, in genomics one can use RNA expression of genes across a tissue sample set. Such data can also be generated or synthesized from the underlying network topology if the topology is the primary structure of interest. Conversely the network topology may be inferred from the sample data if no prior network is known. The node weightings may be interpreted as defining samples from the joint distribution of random variables associated with the nodes.

A natural model for distributions is the Gaussian mixture, used in many data processing and analysis applications. In general, a mixture model is a weighted linear combination of distributions where each component represents a subpopulation. In particular, the Gaussian mixture model (GMM) is a weighted average of Gaussians. GMMs are popular due to their versatility and overall simplicity in data representation. They are ubiquitous in statistics, hypothesis testing, decision theory, and machine learning. The idea is that real-world data may not be densely distributed on a high dimensional space, and instead is concentrated in a low dimensional subspace. Further, in many cases of interest, the data is sparsely distributed into a number of subgroups, and so differences within a given subgroup are not as important as those among the subgroups. Mixture models capture these properties, and this motivated the work of Chen et al. IEEE Access 7, 6269-6278 (2019) to modify optimal mass transport (OMT) theory (C. Villani, Topics in Optimal Transportation (American Mathematical Society, 2003), vol. 58, C. Villani, Optimal Transport: Old and New (Springer, 2008), vol. 338) into a form suitable for Gaussian mixture models.

OMT provides the Gaussian mixture framework with a natural comparison metric between mixtures, and conversely mixtures provide a natural model with which to make the computation of OMT tractable. GMMs were used to model the functional role played by a node with respect to the data along its neighbors in the network. This role is quantified by the average GMM/OMT distance between two nodes, which is called the Gaussian Mixture Transport (GMT) distance. Hierarchical clustering then provides a simplified version of the network for each given level of complexity. The simplified or compressed network represents a projection of the prior network (rather than a subnetwork) which is most relevant according to the evidence observed in the data.

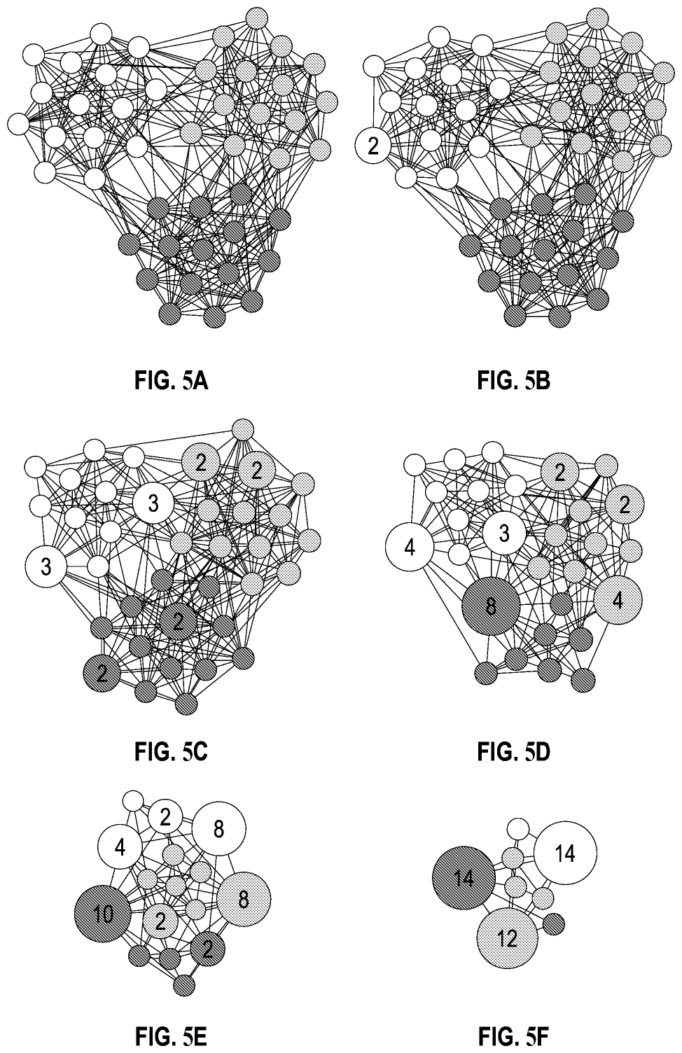

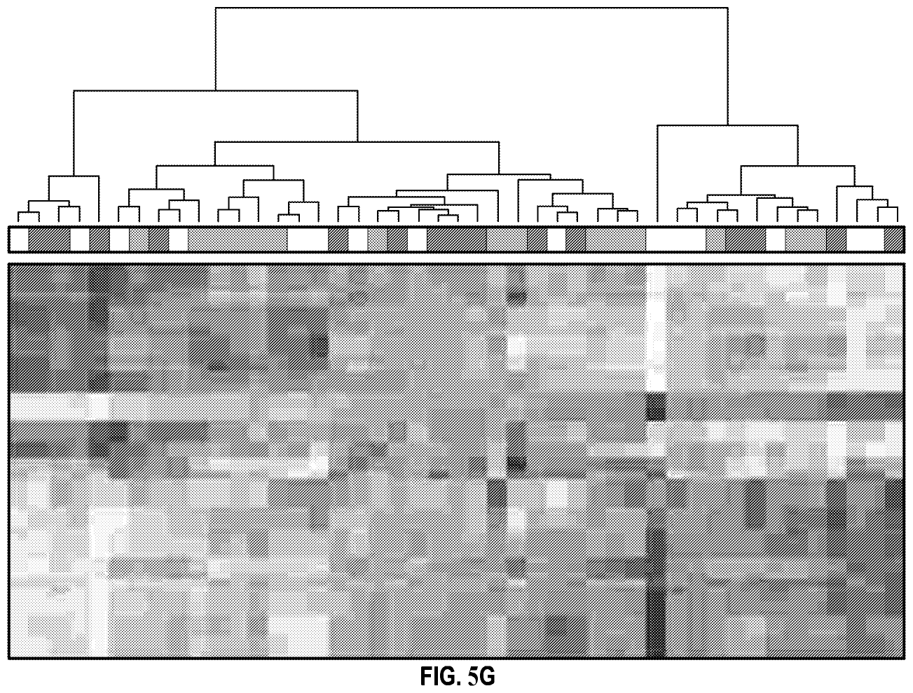

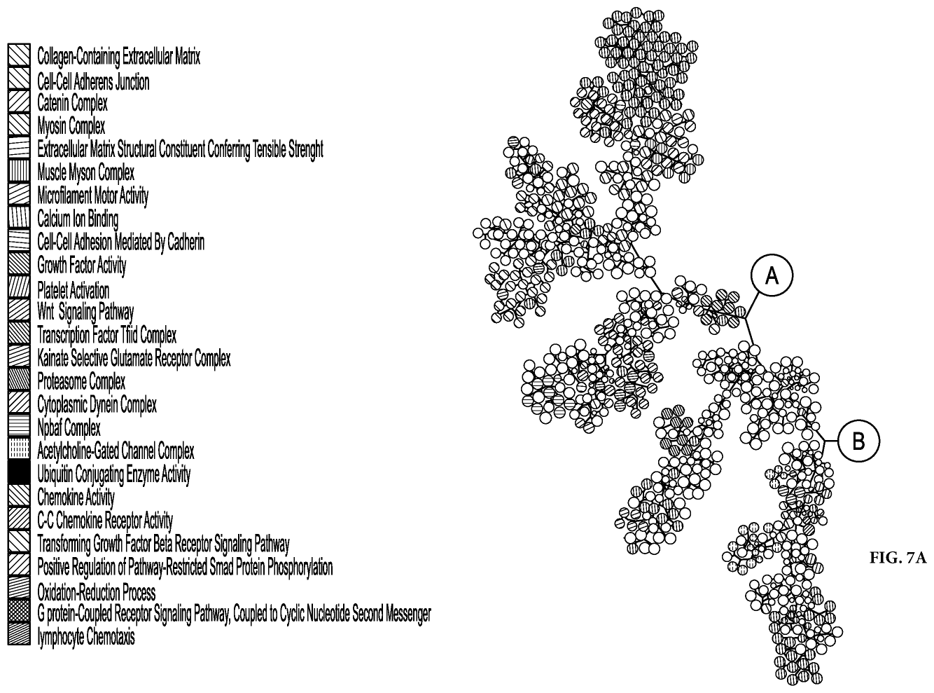

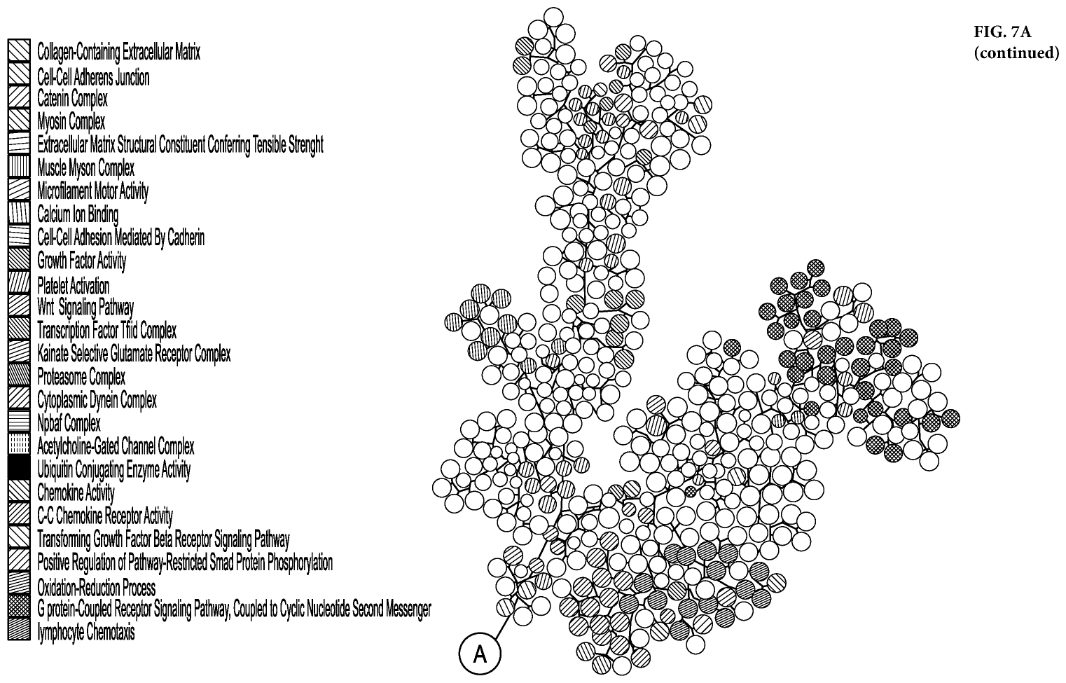

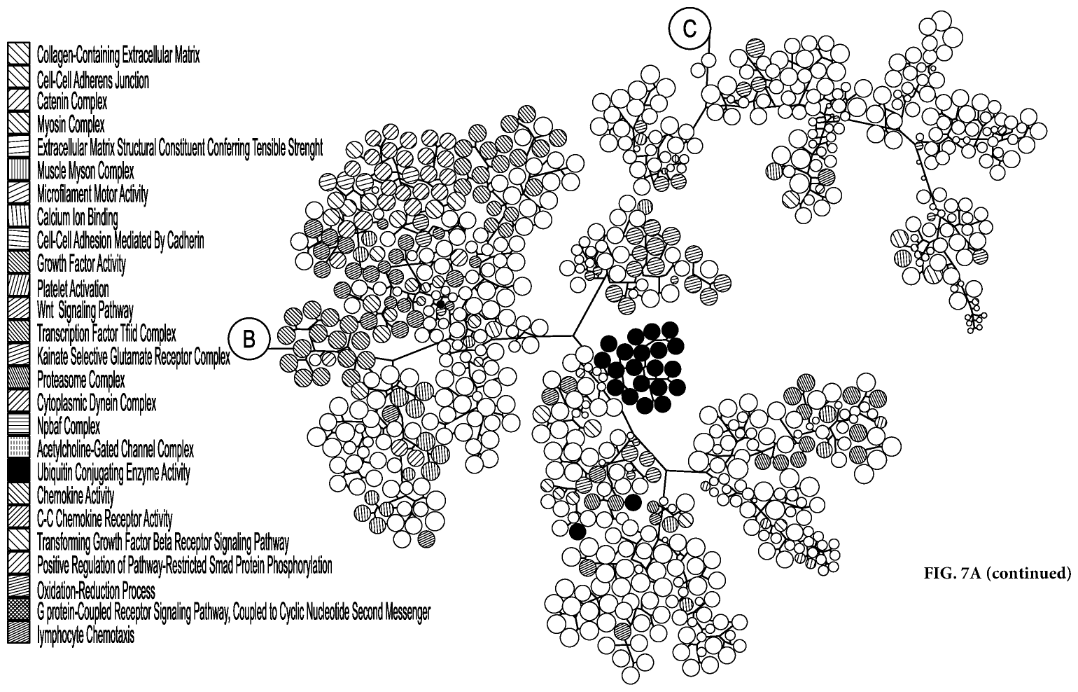

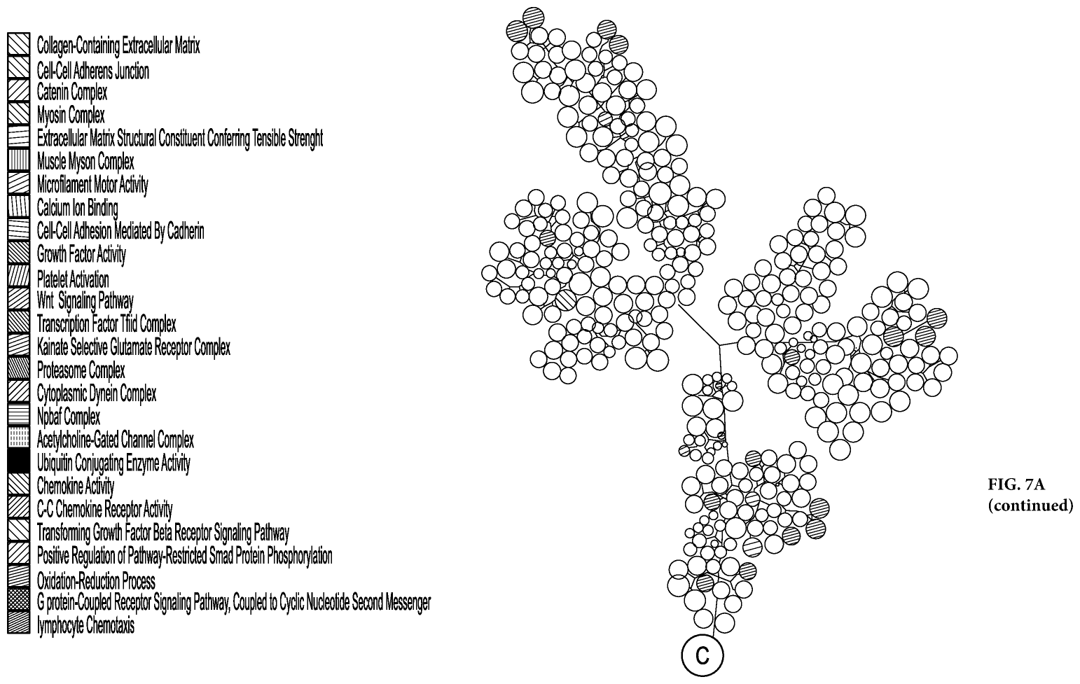

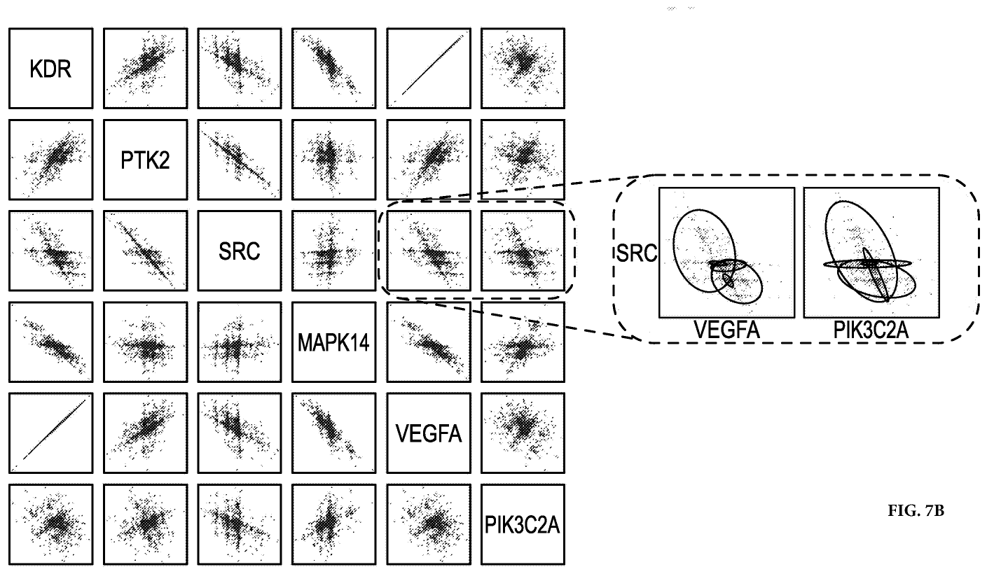

To illustrate the construction of GMT distance-based network reductions, the steps of the construction were applied to a concrete example, and a network was synthesized in order to have a known community structure. The edge density is high within the communities, and low between communities. Node weightings were randomly generated in terms of the network topology by neighbor averaging or the iterated graph Laplacian (Chung & Graham, Spectral Graph Theory (CBMS Regional Conference Series, Conference Board of the Mathematical Sciences, 1997)) of random weightings. An example of such a weighting depicted in FIGS. 7A and 7B. A preview of the series of network simplifications is shown in FIGS. 5A-5G.

Background on OMT for Gaussian mixture models. A Gaussian mixture model is an important instance of the general mixture model structure, a structure that is commonly utilized to study properties of populations with several subgroups (McLachlan & Peel, Finite Mixture Models (John Wiley & Sons, 2004)). Formally, a Gaussian mixture model (GMM) is a probability density consisting of weighted linear combination of several Gaussian components, namely

μ=q¹π¹+q²π²+ . . . +q^{P}π^{P},

where each π^{k} is a Gaussian distribution and q=(q¹, q², . . . , q^{P})^{T} is a probability vector.

Here the finite number P stands for the number of components p.

Let μ₀, μ₁ be two Gaussian mixture models of the form

μᵢ=qᵢ¹πᵢ¹+qᵢ²πᵢ²+ . . . +qᵢ^{P}^{₁}πᵢ^{P}^{₁}, i=0,1.

The distribution μᵢ is equivalent to a discrete measure qᵢ which supports πᵢ¹, πᵢ², . . . , πᵢ^{P}^{₁} for each i=0, 1. The framework from (1) is based on the discrete OMT problem.

$${min}_{π ∈ I I ( {q}_{0} , {q}_{1} )} {\sum }_{i , j} c ( i , j ) π ( i , j ) \qquad [ 1 ]$$

for these two discrete measures, where Π(q₀, q₁) denotes the space of joint distributions with marginal distributions q₀ and qᵢ. The cost c(i, j) is taken to be the 2-Wasserstein metric:

c(i,j)=W₂(π₀ⁱ,π₁^{j})

There is a closed formula for this metric:

$${{W}_{2} ( π , \tilde{π} )}^{2} = {‖ m - \tilde{m} ‖}^{2} + trace ( \sum + \tilde{\sum } - 2 {( {\sum }^{{\frac{1}{2}}_{\tilde{\sum }}} {\sum }^{\frac{1}{2}} )}^{\frac{1}{2}} ) \qquad [ 2 ]$$

where π and \tilde{π} are Gaussian distributions with means m and \tilde{m}, and covariances Σ and \tilde{Σ}, respectively. The discrete OMT problem (Chen et al., IEEE Access 7, 6269-6278 (2019)) always has at least one solution, and letting π* be a minimizer,

$$GMM / OMT Distance ( {μ}_{0} , {μ}_{1} ) = \sqrt{{\sum }_{i , j} c ( i , j ) π * ( i , j )} . \qquad [ 3 ]$$

This formula is a key formula underlying the algorithm employed in various potential embodiments.

Gaussian Mixture Transport distance. A naive approach would group nodes together based on similar properties, for example, by making comparisons between the (univariate) distributions of the weight data associated with each node. Comparison in this case is a classical topic, addressed, for instance, by the Kolmogorov-Smirnov test. One step beyond this is to summarize the joint (bivariate) distribution associated with each edge node-pair by a Pearson correlation or related metric, and then use this similarity metric for classical clustering.

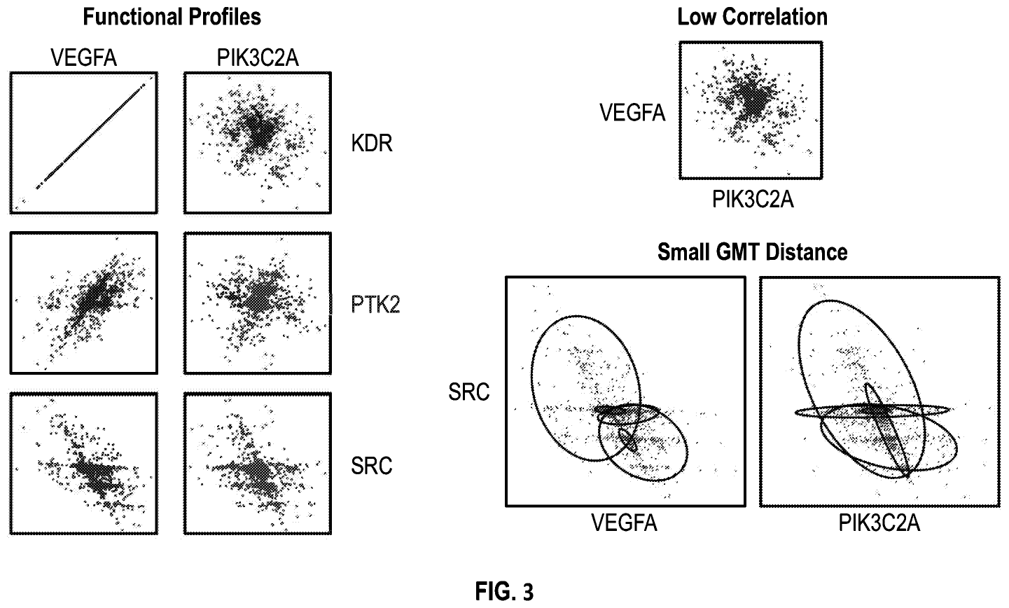

A higher order method is described herein, in which the bivariate distributions associated with each edge (i.e., the joint distributions of the variables associated with the two endpoint nodes) were considered, and then pairs of bivariate distributions associated with adjacent edges X-Z and Y-Z (here the edges are adjacent along node Z) compared. If the distributions are similar, X and Y will be considered to have a similar function in the network locally near Z. Ultimately this similarity over all Z intermediate between X and Y were summarized. FIG. 3 shows an example of similar functional profiles. In this way local parallelism was captured, where some closely related nodes may provide alternative paths to similar effects.

The similarity between bivariate distributions was quantified. The Bhattacharyya distance (Georgiou et al., Lin. Algebra Appl. 425, 663-672 (2007)) is one similarity measure between distributions in higher dimensions. However, a direct calculation of this distance tends to require rasterization of the space involved and may be prohibitively costly to compute. The theory developed by Chen et al. (2019) for Gaussian Mixture Models provides a nearly closed-form alternative which is well-suited to the task, a metric called Gaussian Mixture Transport similarity. First, the distributions were approximated by Gaussian mixtures with a given number of subpopulations. The mixture weights were interpreted as a probability distribution on the discrete set of subpopulations, which are themselves compared using the optimal mass transport metric or discrete Earth Mover's Distance (EMD). For this calculation of the EMD, the cost function corresponding to motion from the discrete point labeling a subpopulation of the first mixture to a discrete point labeling a subpopulation of the second mixture is taken to be the actual optimal mass transport distance between the corresponding Gaussian distributions. The GMT distance between two nodes (not necessarily connected by an edge) is this GMM/OMT distance averaged over all adjacent edges with the same free endpoints. For a detailed description, see Table 2 summarizing the formulae of Chen et al. (2019) and the OMT Background section. The last step is classical hierarchical clustering using the GMT distance sparse similarity matrix. The average-distance-based hierarchical clustering method was used, though the standard alternatives single-linkage, complete-linkage, Ward clustering, etc. may be used depending on the application.

Node data synthesis. Although the GMT hierarchy is primarily designed for reducing the feature structure of a numerical dataset, it can also be applied to a pure network topology by means of synthesized node weights. For this, the iterated graph Laplacian Δ (Chung & Graham (1997)) applied to single-node weightings with randomly chosen support nodes was used. The resulting weightings can be understood as random linear combinations of the A eigenfunctions, with emphasis on those eigenfunctions with large eigenvalues. The spectrum of A is well-studied and known to capture a lot of detailed information about the underlying graph.

Implementation and runtime complexity. A naive version of the algorithm disclosed herein would iterate over all edge pairs, with complexity class O(E²) where E is the number of edges of the network. However, since only adjacent edge pairs are used, the algorithm instead iterated over the nodes and then over the pairs of its neighbors, with complexity class O(ND²) where N is the mixture of nodes and D is the maximum degree over all nodes.

The mixture modeling and GMT distance calculations are classically parallelizable. The mixture modeling, because it only depends on the variable pair distributions, and the GMT distances because they only depend on the resulting list of mixture models. This makes the algorithm feasible for rapid computation. The discrete Earth Mover's Distance is performed with the R package emdist (Urbanek, Y. Rubner, emdist: Earth mover's distance. https://cran.r-project.org/web/packages/emdist/. Accessed 12 Dec. 2018). The mixture modeling itself is performed with the R package mclust (Scrucca et al., R J. 8, 205-233 (2016)). In practice, the number P of mixture model populations has little effect on the overall output and performance as long as P lies in the approximate range from 3 to 10. If the number of node weighting samples M is as low as a few hundred (M=100), it is not meaningful to choose P much greater than 10 anyway, since the number of data points per population (M/P≈10) should not be too low. High accuracy of the mixture model as a representation of the joint distribution of two given node variables is not essential for the purpose of inferring distances between the distributions from distances between the models.

Visualization. Once the hierarchy was computed, it was formatted for viewing in the Gephi graph visualization software using a custom plugin. Gephi is used to represent weight data with node size, color, and relative position in force-directed graph layouts. Three different view types were used. One view shows the compressed network at a user-selected level or scale, as depicted in FIGS. 5A-5G.

For larger graphs, a second, static view is used to reduce the computational burden of real-time rendering. For this the hierarchy itself was used and considered as a graph. This visualization method applies to any hierarchical clustering and could serve as a general-purpose alternative to the usual rectilinear branch representation often used to decorate heatmaps. The graph is a tree or union of trees, with leaf nodes representing features and internal nodes representing feature groups. Leaf node sizes were chosen to reflect the linkage height, or hierarchical level, of the first internal node to which it is attached. Lower levels correspond to larger nodes, since nodes joining the tree at a lower level do so on the basis of stronger evidence of coordination with other nodes (smaller GMT distances). Internal nodes are given negligible size. A planar representation with no edge-crossings is possible since the graph consists of trees. A force-directed layout is used to arrange the nodes in a way guided by the tree structure and node sizes.

In the third view the original network topology, modified to include edges added between neighbors-of-neighbors, is visualized directly with edge weighting equal to the GMT distance. To emphasize a particular scale s, a Gaussian transformation of GMT distance with mean s and a chosen bandwidth ε was used. This representation has much of the computational advantages of a single pre-processed static view, but with some additional flexibility for interactive refinement via s and e.

Data Availability. The gene expression data used in this study are publicly available from the TCGA (Weinstein, E. A. Collisson, E. Mills, Nat. Genet. 45, 1113-1120 (2013)) (www.cancer.gov/tcga) via cBioPortal (Cerami, J. Gao, U. Dogrusoz, Cancer Discov. 2, 401-404 (2012)). Cluster analysis published via the Gene Data Analysis Center (GDAC) Firehose (Broad Institute, TCGA genome data analysis center. gdac.broadinstitute.org/. Accessed 5 Nov. 2018) was used. Code for this paper is available on the public repository github.com/MSK-MedPhys-DeasyLab/functional-network-analysis.

### Example 2: Results of Functional Network Analysis

Data analysis with a large number of variables always involves evaluating some kind of similarity between variables. This serves the purpose of finding mechanisms of action in the system, in which similar variables may indicate subsystems working together to accomplish some function. It also serves the purpose of dimensional reduction, reducing the complexity of the analysis by allowing a member of a group of related variables to serve as a proxy for the whole. Practical unsupervised data analysis is often limited to similarity clustering based on standard sample-wise Pearson correlation, because of its ease of computation and straightforward interpretation. Correlation measures the goodness-of-fit of a linear relationship between two numerical variables. The disadvantage is that the comparison between variables is made without reference to other variables that could be essential for identifying related function.

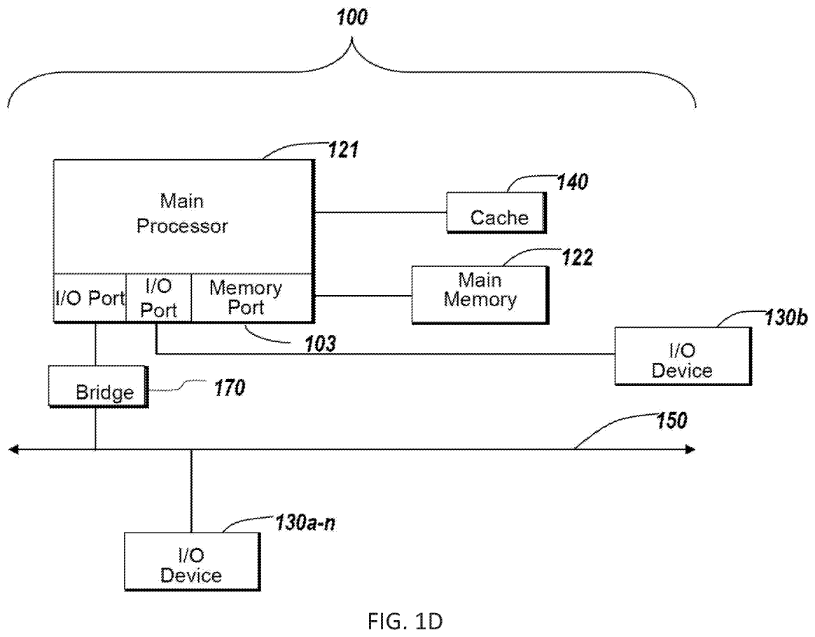

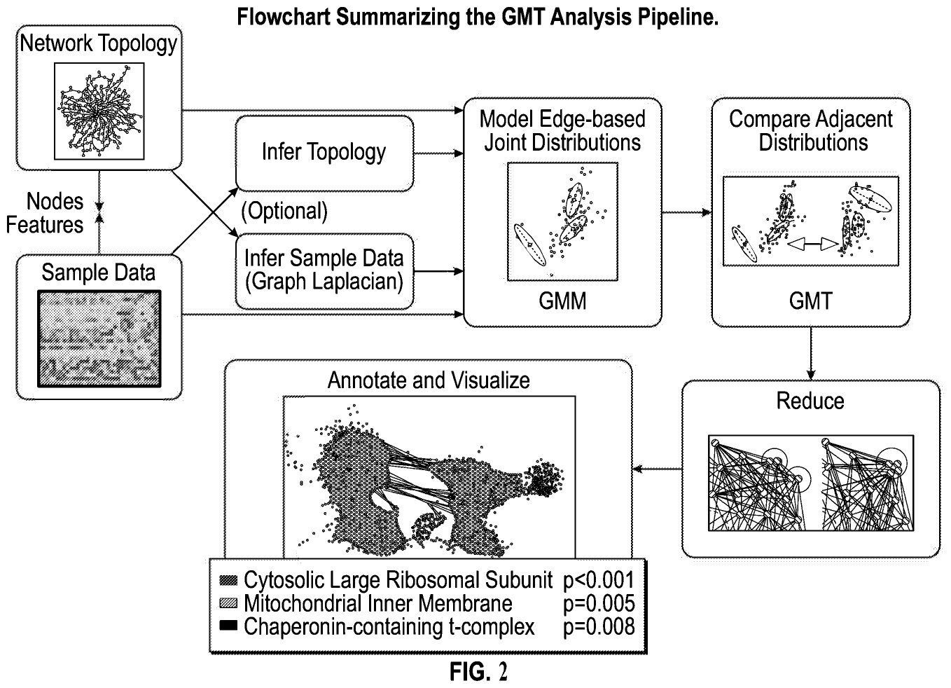

For a higher order approach, rather than directly comparing the values of two variables X and Y, various embodiments compare only the function of the variables in the context of the whole system across a cohort. The idea is that the bivariate distributions associated with each edge were considered, and then pairs of bivariate distributions associated with adjacent edges X-Y and Y-Z were compared. Accordingly the functional profile of a given variable X will mean the collection of joint distributions or scatter plots (X, Z) of X against other variables Z (FIGS. 2-3). To decide which variables Z to use in the functional profile of X, it is assumed that the data set is augmented with a network topology providing abstract connections between variables/features. A variable Z will only be used in the profile of X if there is a connection or edge between X and Z. In some contexts, such as a well-studied molecular pathway, this network may be known a priori and should be considered part of the data set being analyzed (e.g. FIGS. 7A and 7B). In other contexts, such as the large-scale transcriptomic analysis performed on the TCGA lung cancer samples, for an unbiased analysis this network should be data-driven, inferred from the cohort data matrix itself. For network inference, liberal thresholds on the absolute value of the correlation between variables X and Y were used to decide whether X and Y will be connected by an edge.

One often finds by informal investigation that some variables X and Y have common functional profiles even when X and Y are completely uncorrelated. To promote this type of informal investigation to objective analysis, one needs a comparison metric between joint distributions or scatter plots. To this end Gaussian Mixture Models (GMMs) were first fitted to the distributions. This has a smoothing effect, filtering out noise, as well as making the distributions accessible to analytic formulae via the comparatively few fitted parameters. GMMs were selected because they are well-studied and straightforward to fit. A computationally-efficient version of Optimal Mass Transport (OMT) adapted to GMMs is used to measure the distance between the fitted models. Distances based on OMT are (weakly) continuous as opposed to some other commonly used measures of distributions such as Kullback-Leibler divergence and total variation. Further, GMMs are natural models for representing probability distributions. Under very general conditions, probability density functions may be approximated (e.g., in L¹) by such weighted sums of Gaussians.

The final Gaussian Mixture Transport (GMT) distance between X and Y is calculated as the average GMM/OMT distance between the functional profiles of X and Y along variables Z that are common neighbors of X and Y. The GMT metric may be used thereafter as the input to hierarchical clustering algorithms. Thus the GMT metric was employed to create a force-directed graphical representation of the feature network in which close nodes are likely to share a common function with respect to other nearby nodes.

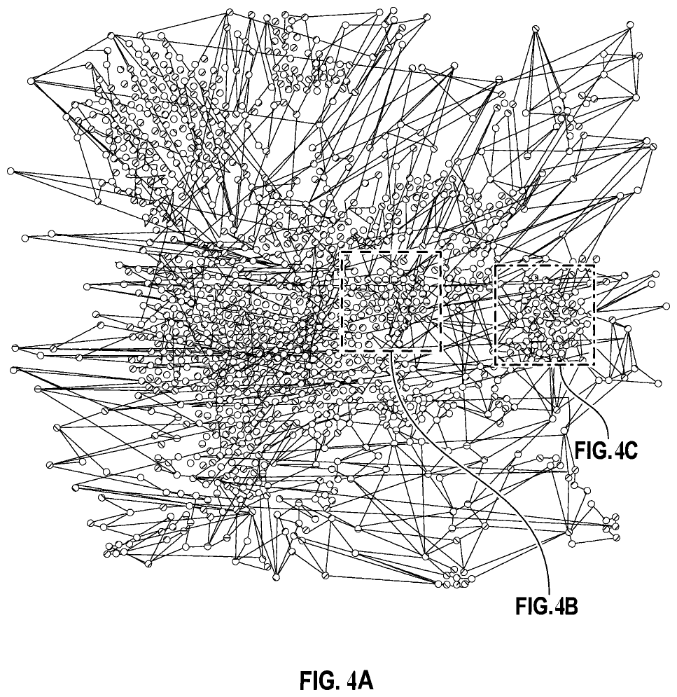

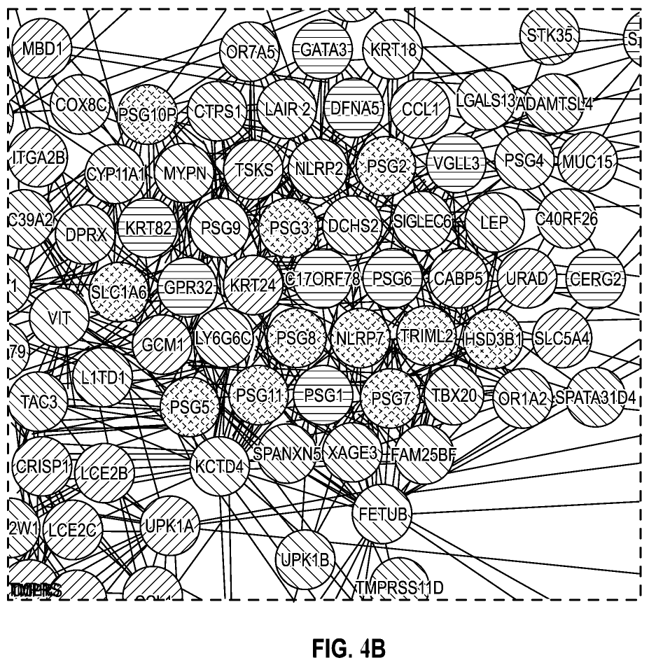

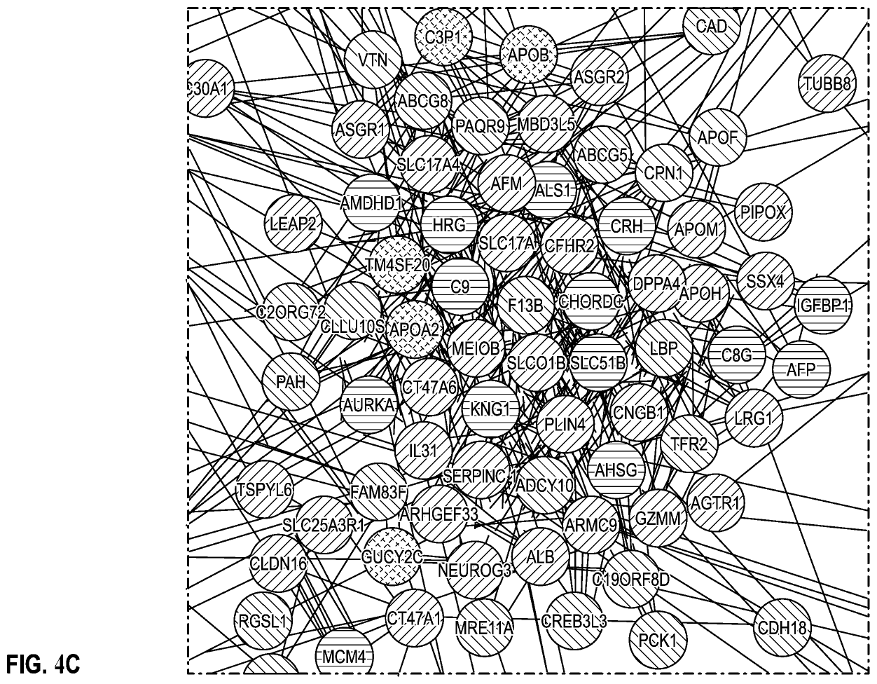

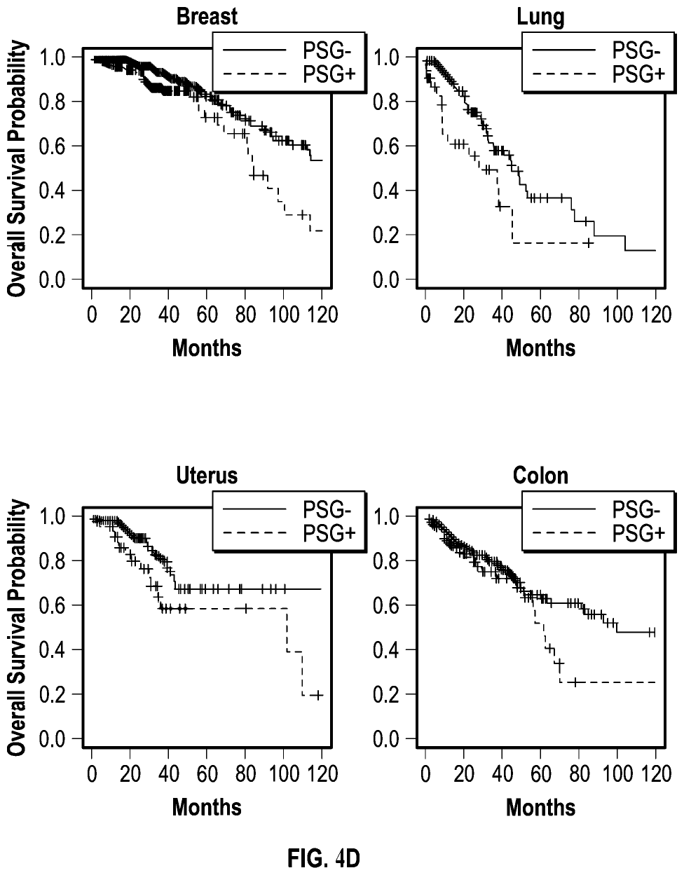

This analysis and visualization methodology is well-suited to hypothesis generation in biological and medical applications with large gene-level data sets. In an investigation of mRNA expression data of lung adenocarcinoma samples, the graphical representation strongly grouped together a module of genes which were further singled out, among all modules found, for their comparatively high expression in a subsample belonging to a published unsupervised cluster with approximately 20% frequency (FIGS. 4A-4D). This module turned out to include all the known 10 Pregnancy-Specific Glycoproteins (PSGs) and several other genes known for expression in the placenta. This suggested the hypothesis that the PSG+ status is related to prognosis, which is confirmed by Kaplan-Meier analysis in the TCGA lung adenocarcinoma, breast, uterine corpus endometrial carcinoma, and colon adenocarcinoma cohorts. Approximately 20% of each cohort have PSG+ tumors, and these have an especially poor prognosis. Together with documented findings relating PSGs to regulation of the immune system, these results implicate the PSGs as a mechanism that mediates immune tolerance in cancers.

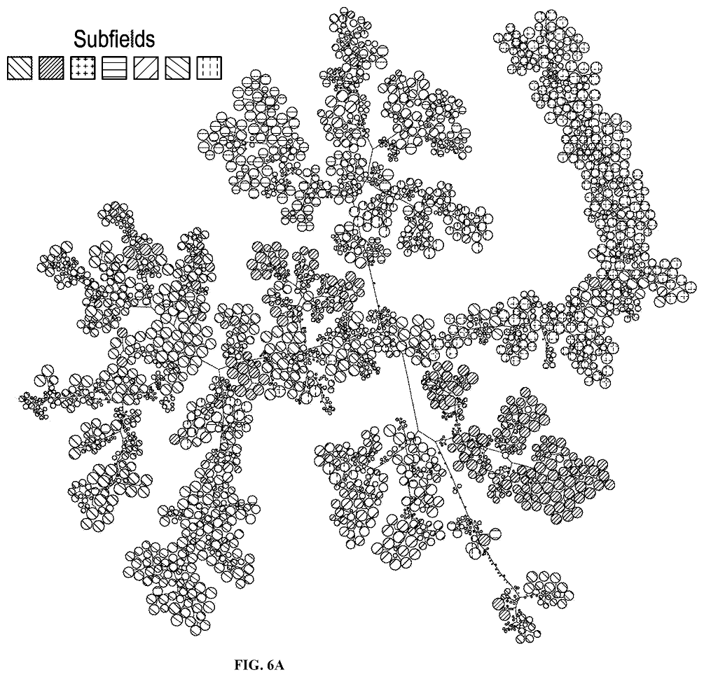

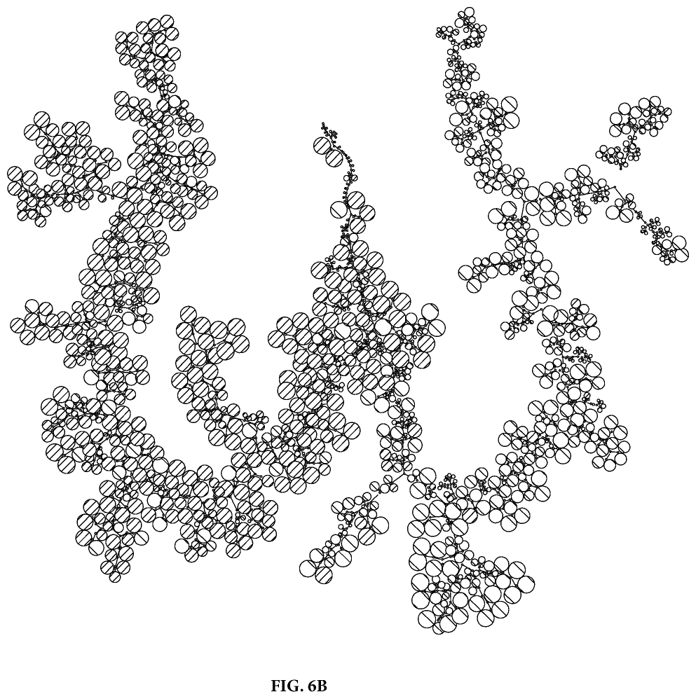

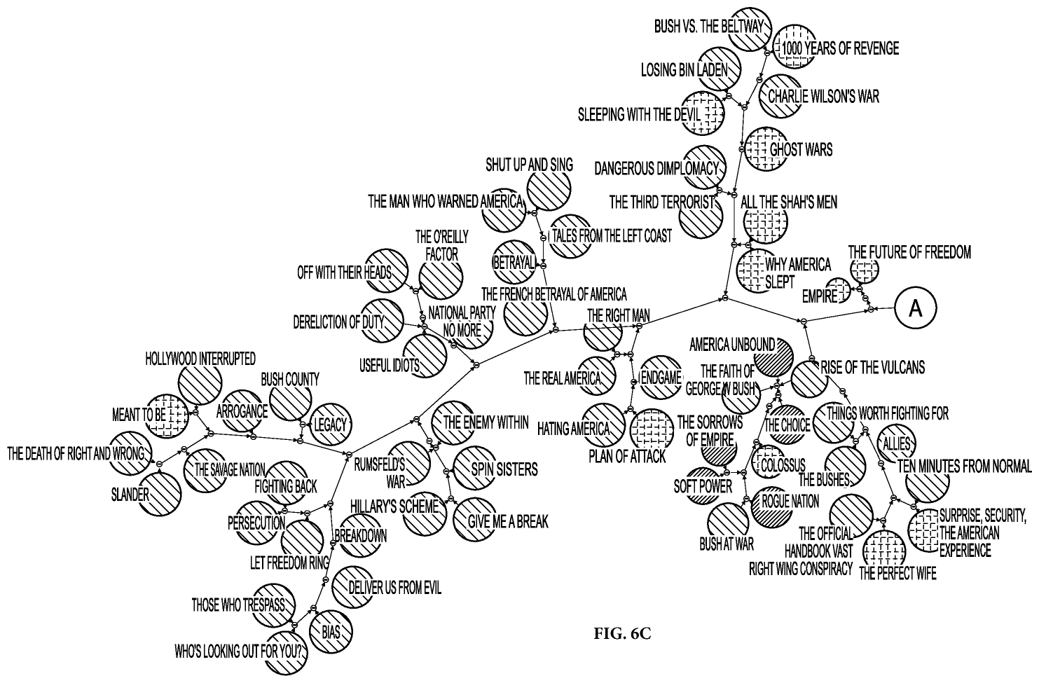

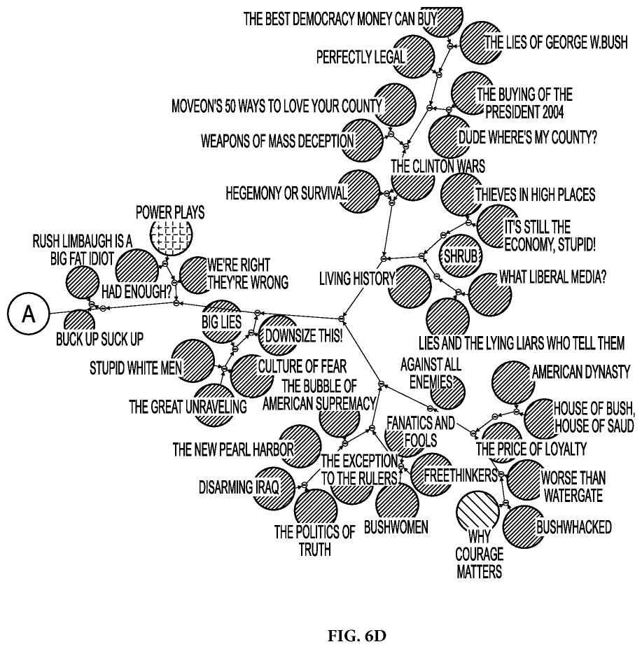

Benchmarking. GMT-based hierarchical clustering can be completed to an unsupervised community detection algorithm using a numerical metric of modularity in the usual way, by selecting from among the level-cutoff clusterings the one with the best value of the metric. In the supervised setting, the level cutoff can be selected to give the best value of a cluster similarity metric, such as Normalized Mutual Information (NMI) calculated against a known community structure. FIG. 6 compares this GMT community detection method with three established methods available in the R igraph library: greedy optimization, Louvain optimization, and label propagation.

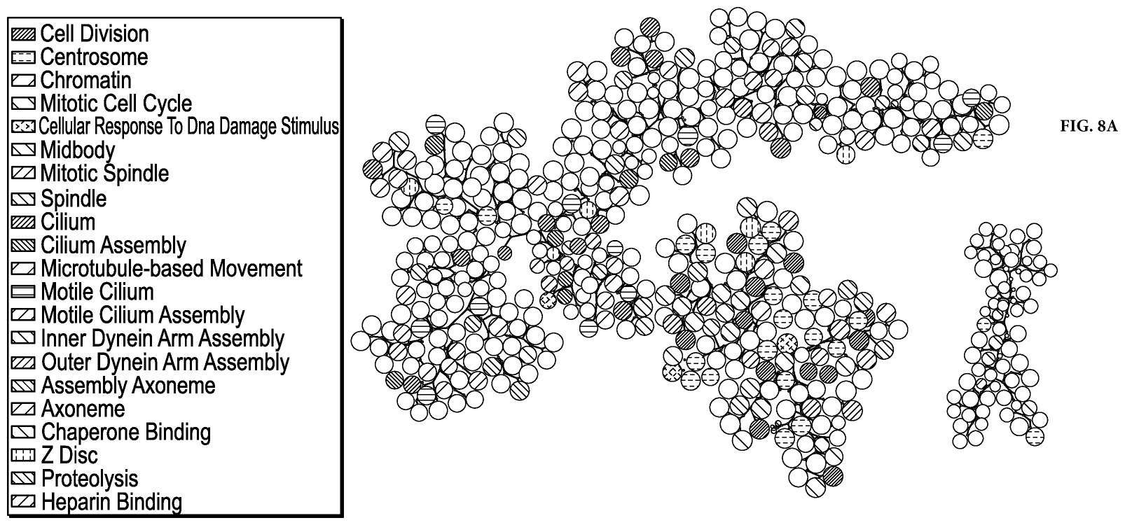

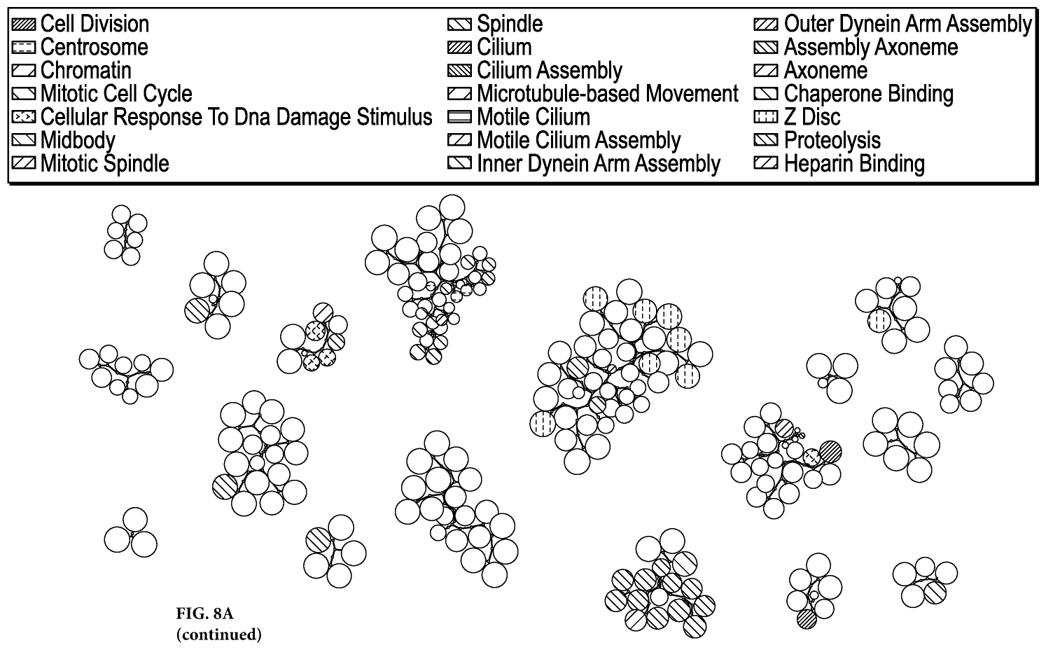

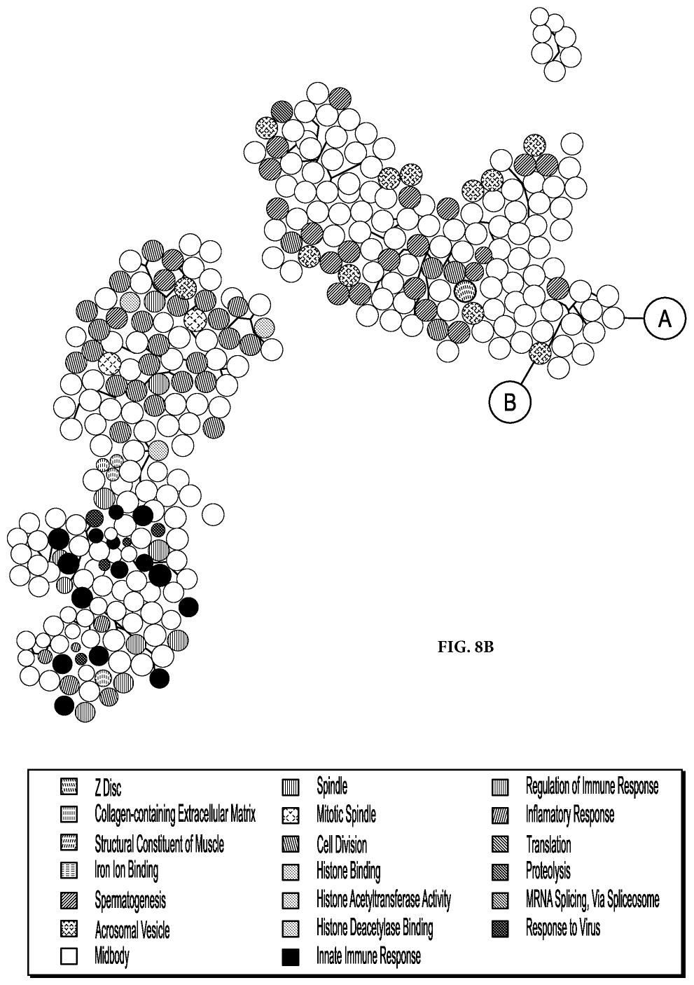

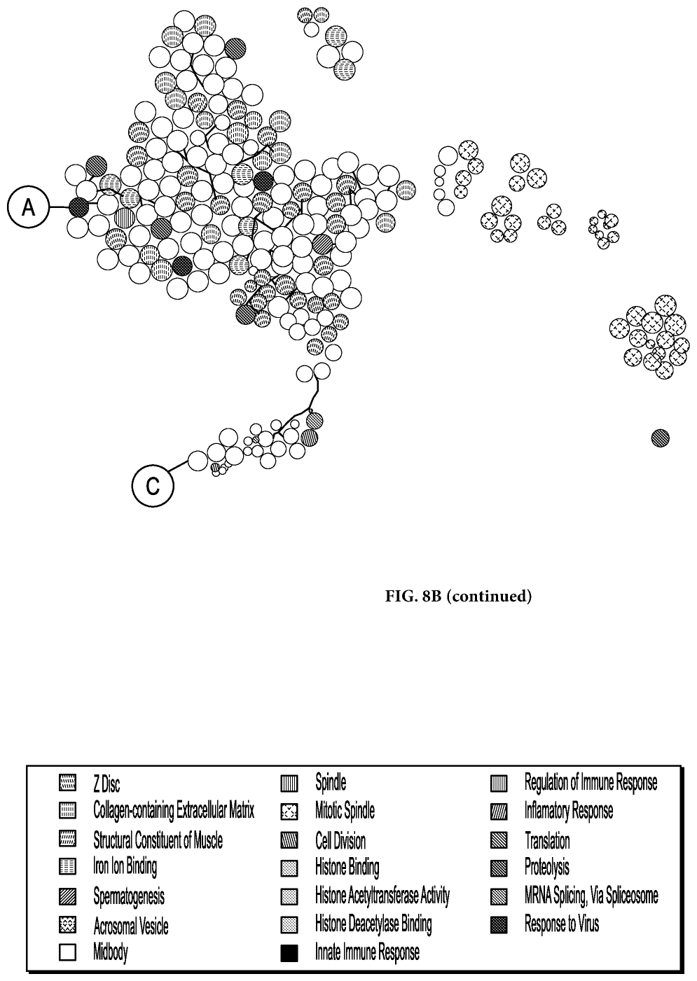

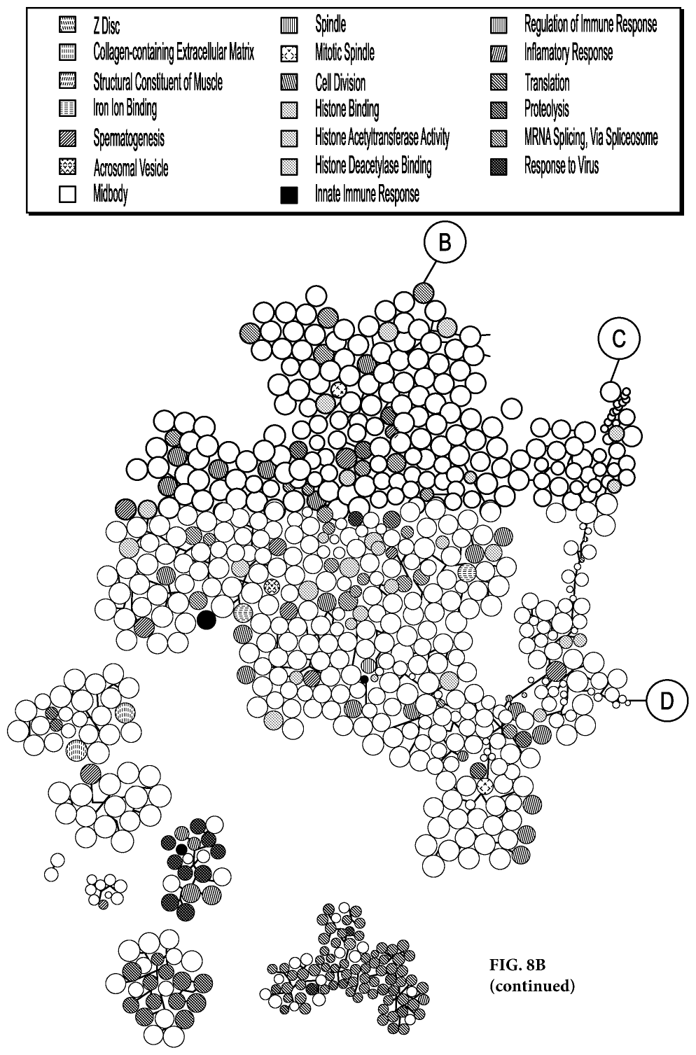

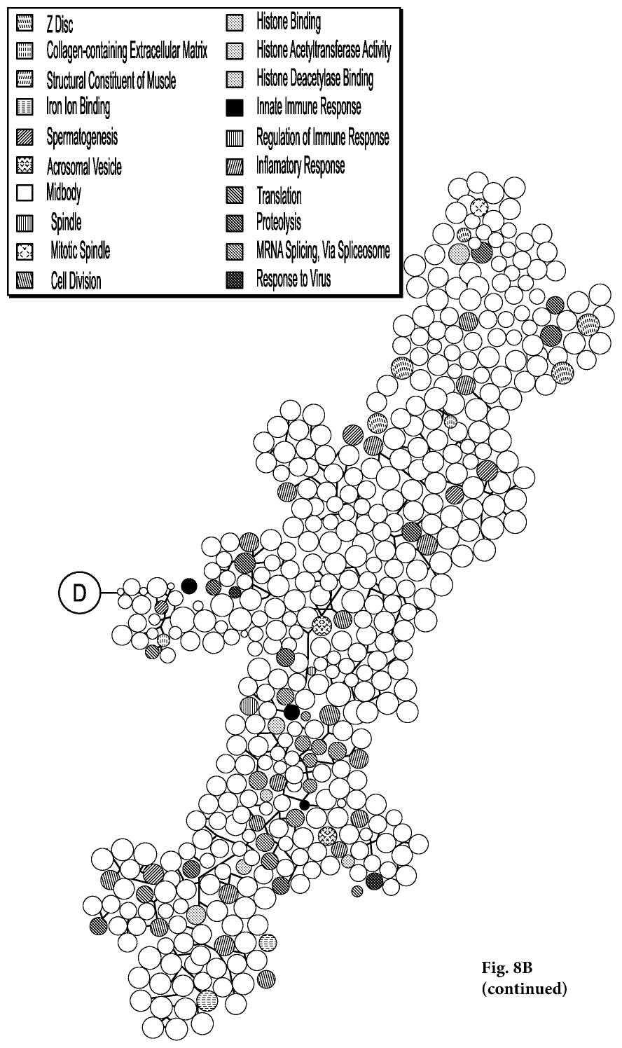

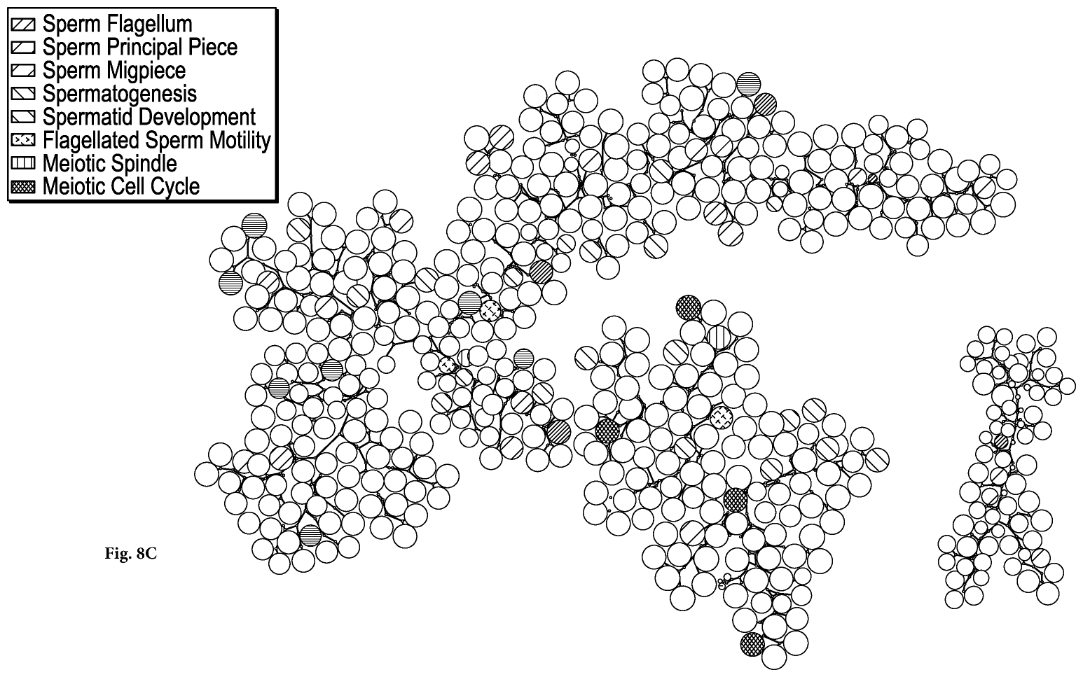

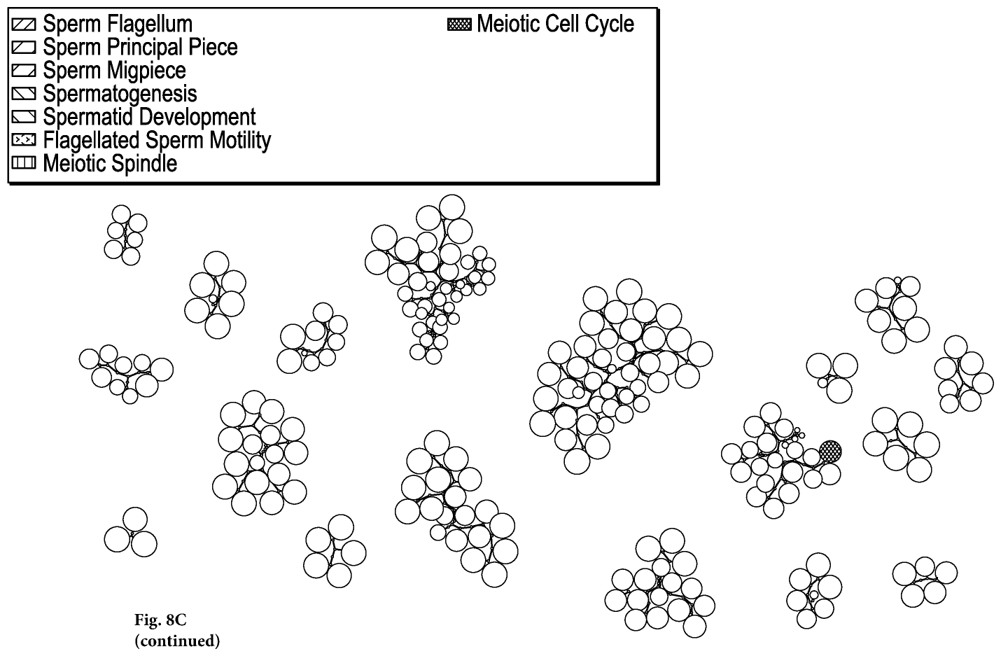

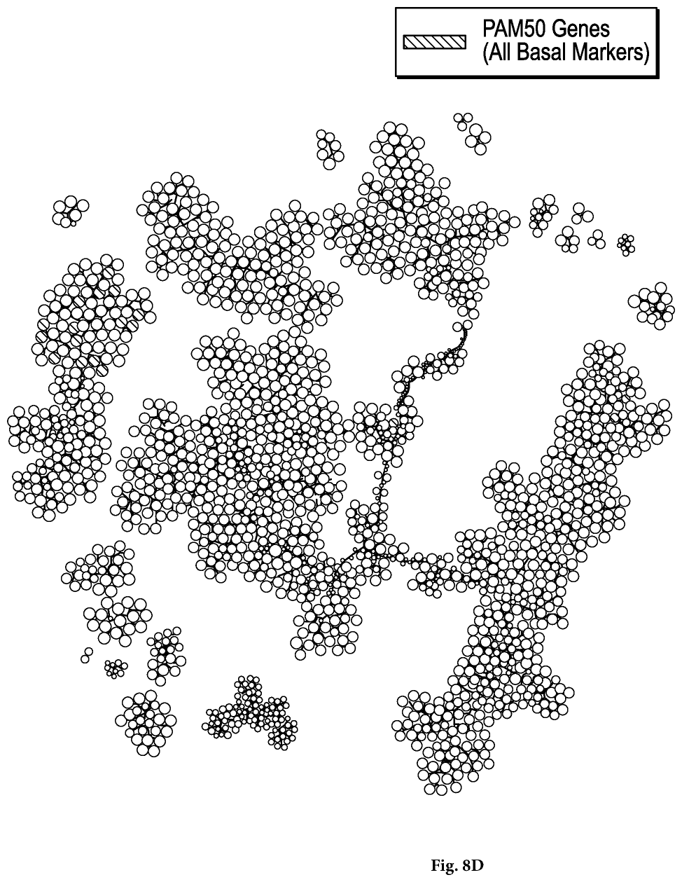

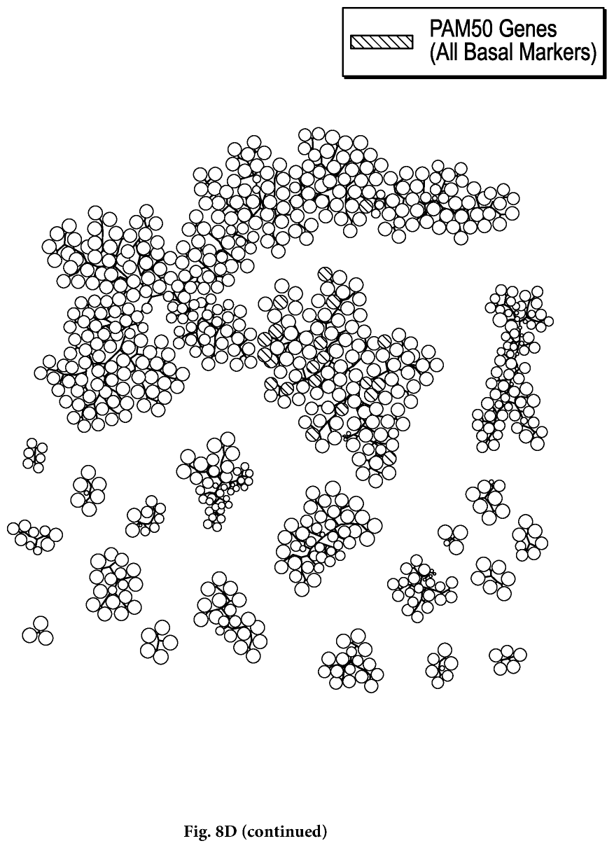

Illustrative GMT analyses. FIGS. 7A and 7B show the results of the GMT analysis on the PANTHER curated gene network. FIG. 8 shows the results on the GTEx (Genotype-Tissue Expression) lung and breast tissue RNA expression datasets. The GTEx analyses take advantage of the full generality of the method by involving non-synthesized node data measurements. In these examples the network topology was inferred from the sample data with a Pearson correlation cutoff, but if a prior network of interest is available it can be used instead. Experience with curated networks (NetPath, PANTHER, HPRD) suggests that the choice of prior network strongly influences the results, the greater the sparsity the stronger the influence. So analyses involving empirical node weights and a prior network should always be compared with a control analysis based on synthetic node weights as in FIGS. 7A and 7B.

Gene Ontology (GO) annotations may be used to explain gene clusters. The genes involved in a cluster come from an unbiased process of gene assembly and the mathematics that is being used by this approach. In fact the GO terms used belong to a separate structural hierarchy tree that goes from a general to a specific description of functions. The annotations that are used to identify a gene in a hierarchy are simply too numerous to be useful. Because of this only those identifiers of gene sets that are well clustered along the hierarchy, based upon the average pairwise graph distance between nodes in the hierarchy, are used. The annotations are assessed for statistical significance as follows. For each tagged subset of genes (i.e., each annotation term), the mean of the node-to-node pairwise weighted graph distances is calculated and regarded as a dispersal or coordination statistic. The statistical significance is measured with 10000-trial bootstrapping by random permutation of the whole gene set, with a p-value recording the fraction of the trials in which the statistic was lower than the observed value. FIGS. 7-8 show the application of this method to the PANTHER curated database of gene interactions and the GTEx gene expression profiles of normal lung and breast tissue.

Tables 1, 2, and 3 show the details of the Gaussian Mixture Transport calculations in algorithmic form.

TABLE 1
Notation for algorithms

| | |
|---|---|
| N | number of nodes |
| E | number of edges |
| S | number of node weightings |
| s | source function [1, E] → [1, N] |
| t | target function [1, E] → [1, N] |
| w | node weight matrix [1, N] × [1, S] → [symbol] |
| P | Gaussian mixture modeling population number |
| GMM | Gaussian mixture model, mclust R package (25) |
| j ~ i | nodes i and j are neighbors |

TABLE 2
GMT distance node similarities algorithm

```
function GMT DISTANCES(s,t,w,P)
  for e in [1, E] do
    Model(e) =GMM(w(s(e), −), w(t(e), −), P)
  end for
  list = { }
  for i in [1, N] do
    for j ~ i do
      for k ~ i, k > j do
        M1 = Model(e(j, i))
        M2 = Model(e(k, i))
        d =GMM/OMT Distance(M1,M2)
        list ← ((j, k), d)
      end for
    end for
  end for
  groups = group(list) by (j, k) value
  similarities = { }
  for group (j, k) in groups do
    similarities(j, k) =average d over group
  end for
  return similarities
end function
```

TABLE 3
GMM/OMT (GMT) Distance algorithm

| | |
|---|---|
| function GMM/OMT DISTANCE(M1,M2) | [symbol] Vector size P |
| D1 = probabilities(M1) | [symbol] Vector size P |

```
  D2 = probabilities(M2)
  for a in [1, P] do
    for b in [1, P] do
      G1 = Gaussian model(M1, a)
      G2 = Gaussian model(M2, b)
      cost(a, b) = OMT distance(M1(a),M2(b))
    end for
  end for
  return Earth Mover's Distance(D1, D2, cost)
end function
function OMT DISTANCE(G1,G2)
  υ₁ = mean(G1)
  υ₂ = mean(G2)
  Σ₁ = covariance matrix(G1)
  Σ₂ = covariance matrix(G2)
  D = sum of squared entries of υ₁ − υ₂
  D′ = trace(Σ₁ + Σ₂ − 2(Σ₁^{1/2}Σ₂Σ₁^{1/2})^{1/2})
  return (D + D′)^{1/2}
end function
```

Analysis of Gene Regulatory Networks. Gene and protein networks are ubiquitous in medicine and biology. Though they are thought to be highly orchestrated, they are complex, and data-driven validation of known pathway mechanisms is still needed.

Functional network analysis of the lung adenocarcinoma RNA expression profiles in the TCGA database, using the GMT metric, produces a module of genes/nodes that are clustered together containing two sets of functionally related genes (FIGS. 4A-4D). One set is expressed by trophoblasts, which reside only in the placenta (during pregnancy) and a second set belonging to the Testis Antigen family of genes. In FIGS. 4A-4D, genes with high levels of transcription as well as genes with low or no detectable levels of transcription are indicated. The quantification of these levels of mRNAs is from the average z-score profile of one sample cluster from the data published by the Broad GDAC Firehose. The genes that are labeled as high levels are largely from the placenta with all eleven genes of the pregnancy specific glycoprotein cluster of genes (PSGs) scoring as actively transcribing. The genes that are labeled as little or no transcription are enriched for the testes specific antigens with only a few genes being expressed.

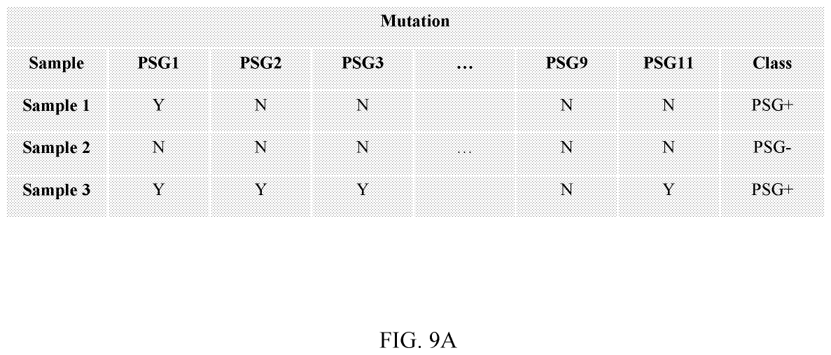

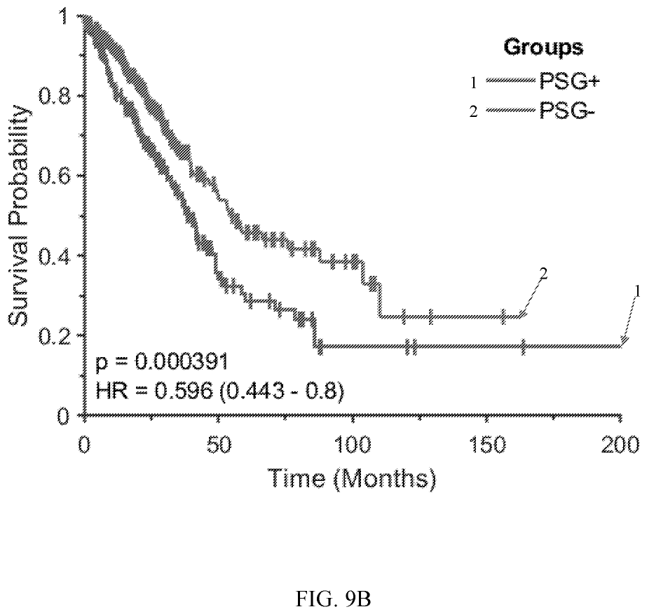

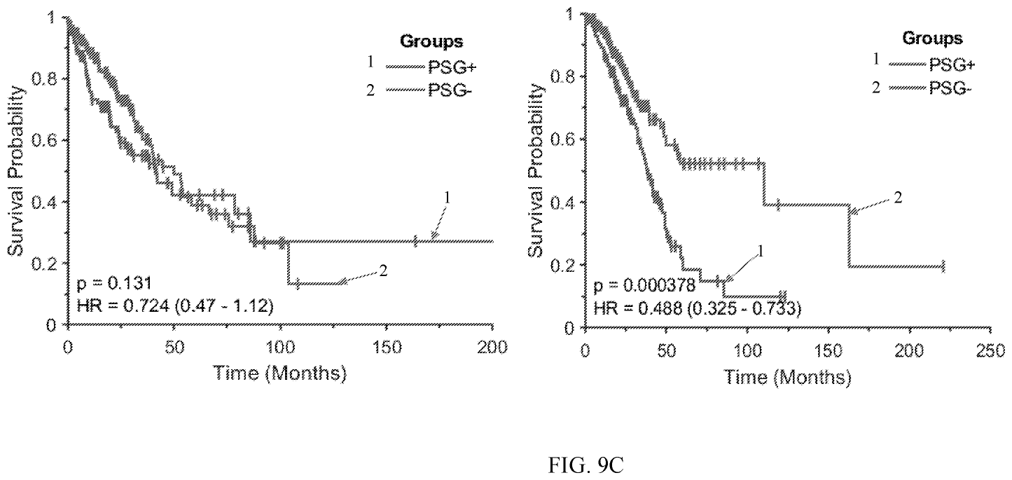

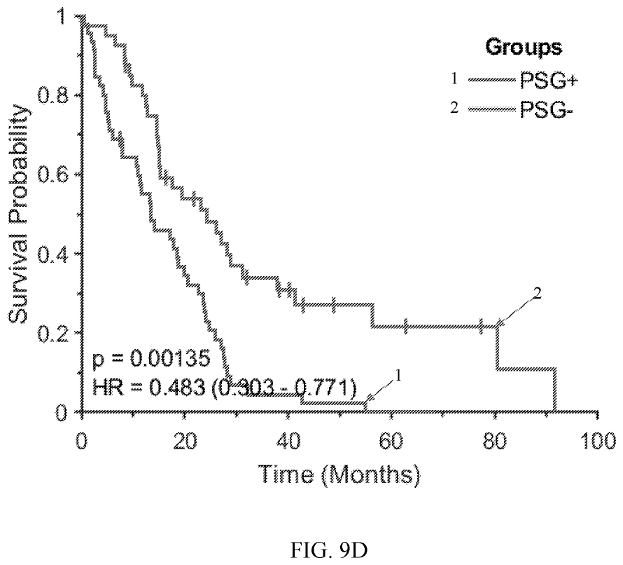

Thus the GMT metric identified a functional set of genes, the PSG genes, which are transcribed only in normal trophoblasts during pregnancy and whose transcripts were also detected in about 20% of lung adenocarcinomas in the TCGA. Furthermore those cancers that expressed the PSG genes had the worst overall survivals as tested in Kaplan-Meier plots with statistical significance (FIGS. 4A-4D). Similar results detecting the transcription of the PSGs genes in about 20% of cancers of the breast, uterus, and colon were also observed. These correlations were independent of patient gender. See also FIGS. 9A-9D. Rousseaux et al., Sci. Transl. Med. 5, 186ra66 (2013) have shown that adenocarcinomas of non-small cell lung cancers that express PSGs are commonly very aggressive and metastatic. This is consistent with the observations in FIGS. 4A-4D demonstrating that NSCLC adenocarcinomas that express the PSGs have poorer overall survivals than those that do not express the PSGs. Very similar results are observed with breast, colon, uterus and lung cancers. The PSG genes are in a cluster of genes expressed in the placenta and so any of these genes in the module shown in FIGS. 4A-4D may have some negative impact upon immune surveillance resulting in a poor overall survival.

### Example 3: Role of PSG in Negatively Regulating Anti-Tumor Response

There is growing evidence that the PSGs initiate the production of CD-4 (cluster of differentiation 4) T-regs (regulatory T cells) containing CTLA-4 (cytotoxic T-lymphocyte-associated protein 4) that are utilized during pregnancy to effect an immunosuppressive state that prevents the fetus and embryo from being rejected. Without wishing to be bound by theory, it is believed that this same mechanism is utilized to help prevent the immune system from rejecting tumors that also harbor foreign neo-antigens by virtue of the mutations found in cancers.

Antibodies directed against PSGs in cancer patients whose tumors secrete PSGs are anticipated to promote tumor rejection and/or extend the lifespan of the patient. It is believed that PSGs function at least in part to immunosuppress cancer patients from attacking their tumors through the production of CTLA-4 expressing T-regs and that anti-CTLA-4 antibodies would be expected to function to reverse immunosuppression in PSG expressing tumors. One advantage with this approach is that unlike anti-CTLA-4 antibodies, antibodies directed against the PSGs are not anticipated to induce autoimmunity because the PSGs are not expressed in normal tissue and downregulating PSGs in a tumor would have little to no impact upon normal tissue.

These results demonstrate that cancers utilize normal physiological processes to escape immune surveillance and repurpose the PSGs to evade the immune system.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

What is claimed is:

1. A method for treating cancer in a subject in need thereof comprising administering to the subject a therapeutically effective amount of at least one pregnancy specific glycoprotein (PSG) inhibitor.

2. The method of claim 1, wherein the subject comprises at least one tumor that overexpresses one or more PSG genes.

3. The method of claim 2, wherein the one or more PSG genes are selected from the group consisting of PSG1, PSG2, PSG3, PSG4, PSG5, PSG6, PSG7, PSG8, PSG9, PSG10, and PSG11.

4. The method of any one of claims 1-3, wherein the cancer is breast cancer, lung cancer, uterine cancer, colon cancer, rectal cancer, endometrial cancer, stomach cancer, intestinal cancer, renal cancer, acute lymphoblastic leukemia (ALL), chronic lymphocytic leukemia (CLL), acute myeloid leukemia (AML), and metastases thereof.

5. The method of any one of claims 1-4, wherein the at least one PSG inhibitor specifically inhibits one or more PSG genes selected from the group consisting of PSG1, PSG2, PSG3, PSG4, PSG5, PSG6, PSG7, PSG8, PSG9, PSG10, and PSG11.

6. The method of claim 5, wherein the at least one PSG inhibitor is an antisense oligonucleotide, a sgRNA, a shRNA, a siRNA, an aptamer, a ribozyme, an antibody agent, or a small molecule inhibitor.

7. The method of claim 6, wherein the antibody agent is a monoclonal antibody, a human antibody, a humanized antibody, a multi-specific antibody, a bispecific antibody, a camelised antibody, a chimeric antibody, a Fab, a F(ab′)2, a Fab′, a scFv, a Fv, a Fd, a dAB, a single domain antibody (e.g., nanobody, single domain camelid antibody), a scFv-Fc, a VNAR fragment, a bispecific T-cell engager (BITE) antibody, a minibody, an antibody drug conjugate, a fusion polypeptide, a disulfide-linked Fv (sdFv), an intrabody, or an anti-idiotypic antibody.

8. The method of any one of claims 1-7, further comprising administering one or more of chemotherapy, radiation therapy, immunotherapy, anti-cancer nucleic acids or anti-cancer proteins to the subject.

9. The method of claim 8, wherein the immunotherapy comprises one or more of immune checkpoint inhibitor therapy, adoptive cell therapy, cytokines, immunomodulators, cancer vaccines, monoclonal antibodies, and oncolytic viruses.

10. The method of claim 9, wherein the adoptive cell therapy is Tumor-Infiltrating Lymphocyte (TIL) Therapy, Engineered T Cell Receptor (TCR) Therapy, Chimeric Antigen Receptor (CAR) T Cell Therapy, or Natural Killer (NK) Cell Therapy.

11. The method of claim 9 or 10, wherein the cytokine is selected from the group consisting of interferon α, interferon β, interferon γ, complement C5a, IL-2, TNFalpha, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCL9, CXCR1, CXCR2, CXCR4, CXCR5, CXCR6, CXCR7 and XCL2.

12. The method of any one of claims 9-11, wherein the immune checkpoint inhibitor therapy comprises an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, or an anti-LAG-3 antibody.

13. The method of any one of claims 1-12, wherein the at least one PSG inhibitor is administered systemically, topically, intravenously, intramuscularly, intraarterially, intrathecally, intracapsularly, intraorbitally, intradermally, intraperitoneally, transtracheally, subcutaneously, intracerebroventricularly, orally, intratumorally, intraocularly, iontophoretically, or intranasally.

14. The method of any one of claims 1-13, wherein administration of the at least one PSG inhibitor results in one or more improvements in the subject selected among (a) reduced levels and/or activity of CTLA-4 expressing regulatory T cells, (b) inhibition of tumor cell proliferation and/or tumor metastasis, (c) reduced tumor size, (d) amelioration of cancer symptoms, (e) increased weight gain, (f) extended lifespan, (g) prolonged progression-free survival, and (h) decreased risk of cancer therapy-associated side-effects (e.g., autoimmunity).

15. A method for enhancing anti-tumor immune responses in a subject suffering from cancer comprising
(a) detecting an increase in mRNA or polypeptide expression levels of one or more PSG genes in a test sample obtained from the subject relative to that in a reference sample or a predetermined threshold, and
(b) administering to the subject an effective amount of immune checkpoint blockade therapy.

16. A method for enhancing anti-tumor immune responses in a subject suffering from cancer comprising
administering to the subject an effective amount of immune checkpoint blockade therapy,
wherein mRNA or polypeptide expression and/or activity levels of one or more PSG genes in a test sample obtained from the subject are elevated compared to that in a reference sample or a predetermined threshold.

17. The method of claim 15 or 16, wherein the polypeptide expression levels of one or more PSG genes are detected via Western Blotting, flow cytometry, Enzyme-linked immunosorbent assay (ELISA), immunoprecipitation, immunoelectrophoresis, immunostaining, isoelectric focusing, High-performance liquid chromatography (HPLC), or mass-spectrometry.

18. The method of claim 15 or 16, wherein the mRNA expression levels of one or more PSG genes are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH).

19. The method of any one of claims 15-18, wherein the test sample comprises blood, plasma, urine, serum, or tumor tissue.

20. The method of any one of claims 15-19, wherein the immune checkpoint blockade therapy comprises an anti-PD-1 antibody, an anti-PD-L1 antibody, an anti-PD-L2 antibody, an anti-CTLA-4 antibody, an anti-TIM3 antibody, an anti-4-1BB antibody, an anti-CD73 antibody, an anti-GITR antibody, or an anti-LAG-3 antibody.

21. The method of any one of claims 15-20, wherein the one or more PSG genes are selected from the group consisting of PSG1, PSG2, PSG3, PSG4, PSG5, PSG6, PSG7, PSG8, PSG9, PSG10, and PSG11.

22. The method of any one of claims 15-21, wherein the cancer is breast cancer, lung cancer, uterine cancer, colon cancer, rectal cancer, endometrial cancer, stomach cancer, intestinal cancer, renal cancer, acute lymphoblastic leukemia (ALL), chronic lymphocytic leukemia (CLL), acute myeloid leukemia (AML), and metastases thereof.

23. The method of any one of claims 15-22, wherein the reference sample is a non-tumor biological sample obtained from the subject suffering from cancer or a biological sample obtained from a healthy control subject.

24. The method of any one of claims 15-23, further comprising administering chemotherapy, radiation therapy, immunotherapy, anti-cancer nucleic acids or proteins, or combinations thereof to the subject.

25. The method of any one of claims 15-24, wherein anti-tumor responses comprise one or more of increasing levels and/or cytotoxic activity of CD8+ T cells, reducing T cell exhaustion, and/or reduced levels and/or activity of CTLA-4 expressing regulatory T cells.
